SEQUENCE LISTING (14 sequences)

SEQ ID NO: 1 - protein, 297 aa, Escherichia coli
MSAVLQRFREKLPHKPYCTNDFAYGVRILPKNIAILARFIQQNQPHALYWLPFDVDRTGASIDWSDRNCPAPNITVKNPRNGHAHLLYALALPVRTAPDASASALRYAAAIERALCEKLGADVNYSGLICKNPCHPEWQEVEWREEPYTLDELADYLDLSASARRSVDKNYGLGRNYHLFEKVRKWAYRAIRQGWPVFSQWLDAVIQRVEMYNASLPVPLSPAECRAIGKSIAKYTHRKFSPEGFSAVQAARGRKGGTKSKRAAVPTSARSLKPWEALGISRATYYRKLKCDPDLAK

SEQ ID NO: 2 - DNA, 40 bp, synthetic construct
  source 1..40: mol_type = other DNA
agggcgctgttatctgataaggcttatctggtctcatttt

SEQ ID NO: 3 - DNA, 36 bp, synthetic construct
  source 1..36: mol_type = other DNA
agggcgctgttatctgataaggcttatctggtctca

SEQ ID NO: 4 - DNA, 33 bp, synthetic construct
  source 1..33: mol_type = other DNA
gcgctgttatctgataaggcttatctggtctca

SEQ ID NO: 5 - DNA, 12693 bp, synthetic construct
  source 1..12693: mol_type = other DNA
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcggtctcaaggcgctggaagcgcgctttgtgctggaagataagctgattctgctggtgcttgacgccgcccgcgtcaaacatcctgcttgagttctgcgctgttaacgggaaacacagaaaaaagcccgcacctgacagtgcgggctttttttttcgaccaaaggctcggtaccaaattccagaaaagacacccgaaagggtgttttttcgttttggtcccaattattgaaggcctccctaacggggggcctttttttgtttctggtcacccgcttaacgatcgttggctgaacaaacagacaatctggtctgtttgtattatggaaaatttttctgtataatagattcaacaaacagacaatctggtctgtttgtattatagctgtcaccggatgtgctttccggtctgatgagtccgtgaggacgaaacagcctctacaaataattttgtttaatactagagaaagaggagaaatactagatgcgtgcactggttgttattcgtctgagccgtgttaccgatgcaaccaccagtccggaacgtcagctggaaagctgtcagcagctgtgtgcacagcgtggttgggatgttgttggtgttgccgaagatctggatgttagcggtgcagttgatccgtttgatcgtaaacgtcgtccgaatctggcacgttggctggcatttgaagaacagccgtttgatgttattgttgcctatcgtgttgatcgtctgacccgtagcattcgtcatctgcagcagctggttcattgggcagaagatcataaaaagctggttgtgagcgcaaccgaagcacattttgataccaccacaccgtttgcagcagttgttattgcactgatgggcaccgttgcacagatggaactggaagcaattaaagaacgtaatcgtagcgcagcccattttaacattcgtgcaggtaaatatcgtggtagcctgcctccgtggggttatctgccgacacgtgttgatggtgaatggcgtctggttccggatcctgttcagcgtgaacgtattctggaagtttatcatcgtgtggtggataatcatgaaccgctgcatctggttgcacatgatctgaatcgtcgtggtgttctgagcccgaaagattattttgcacagctgcagggtcgtgaaccgcagggtcgcgaatggtcagcaaccgcactgaaacgtagcatgattagcgaagcaatgctgggttatgcaaccctgaatggtaaaaccgttcgtgatgatgatggtgcaccgctggttcgtgcagaaccgattctgacacgtgaacagctggaagcactgcgtgccgaactggttaaaaccagccgtgcaaaaccggcagttagcaccccgagcctgctgctgcgtgttctgttttgtgcagtttgtggtgaaccggcatacaaatttgccggtggtggtcgtaaacatccgcgttatcgttgtcgtagcatgggttttccgaaacattgtggtaatggtacagttgcaatggcagaatgggatgcattttgcgaagaacaggttctggatctgctgggtgatgccgaacgtctggaaaaagtttgggttgcaggtagcgatagcgcagttgaactggccgaagttaatgcagaactggttgatctgaccagcctgattggtagtccggcatatcgtgccggtagtccgcagcgtgaagcactggatgcacgtattgcagcactggcagcacgtcaagaagaattagaaggtctggaagcccgtccgagcggttgggaatggcgtgaaaccggtcagcgttttggtgattggtggcgtgagcaggataccgcagccaaaaatacctggctgcgtagtatgaatgttcgcctgacctttgatgttcgcggtggtctgacgcgtaccattgattttggcgatctgcaagaatatgaacagcatctgcgtctgggtagcgttgttgaacgtctgcataccggcatgagctaactcggtaccaaattccagaaaagaggcctcccgaaaggggggccttttttcgttttggtccaatggcggcgcgccatcgaatggtgcaaaacctttcgcggtatggcatgatagcgcccggaagagagtcaattcagggtggtgaatatgagcccgaaacgtcgtacccaggcagaacgtgcaatggaaacccagggtaaactgattgcagcagcactgggtgttctgcgtgaaaaaggttatgcaggttttcgtattgcagatgttccgggtgcagccggtgttagccgtggtgcacagagccatcattttccgaccaaactggaactgctgctggcaacctttgaatggctgtatgagcagattaccgaacgtagccgtgcacgtctggcaaaactgaaaccggaagatgatgttattcagcagatgctggatgatgcagcagaattttttctggatgatgattttagcatcggcctggatctgattgttgcagcagatcgtgatccggcactgcgtgaaggtattcagcgtaccgttgaacgtaatcgttttgttgttgaagatatgtggctgggtgtgctggtgagccgtggtctgagccgtgatgatgccgaagatattctgtggctgatttttaacagcgttcgtggtctggtagttcgtagcctgtggcagaaagataaagaacgttttgaacgtgtgcgtaatagcaccctggaaattgcacgtgaacgttatgcaaaattcaaacgttgataaggatcctaattggtaacgaatcagacaattgacggctcgagggagtagcatagggtttgcagaatccctgcttcgtccatttgacaggcacattatgcatcgatgataagctgtcaaacatgagcagatcctctacgccggacgcatcgtggccggcatcaccggcgccacaggtgcggttgctggcgcctatatcgccgacatcaccgatggggaagatcgggctcgccacttcgggctcatgagcaaatattttatctgaggtgcttcctcgctcactgactcgctgcacgaggcagacctcaggatctccaggcatcaaataaaacgaaaggctcagtcgaaagactgggcctttcgttttatctgttgtttgtcggtgaacgctctctactagagtcacactggctcaccttcgggtgggcctttctgcgtttataggatctgcgaagttcctatactttctagagaataggaacttcggatctgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatcggatctgcgaagttcctatactttctagagaataggaacttcgccggcttatcggtcagtttcacctgatttacgtaaaaacccgcttcggcgggtttttgcttttggaggggcagaaagatgaatgactgtccacgacgctatacccaaaagaaaaaaaaaaaaacccgcccctgacagggcggggttttttttgcttcaaatgcggatttgacagctagctcagtcctaggtattgtgctagccgtaatttacattcaatgccccatttgcggggctaatttcttgtcggagtgccttaactggctgagaccaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatagccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 6 - DNA, 13186 bp, synthetic construct
  source 1..13186: mol_type = other DNA
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcggtctcaaccaattggcgcgcgcttcgcaataaaatctcccttcgcccagtattgggaaaagtagatacattcaaactgtgtacgctgtttcgtctcacagttcacgacatttaataaaaaaagggcggtcgcaagatcgcccttttttacgtatgacacagtgaaaaatggcgcccatcggcgccatttttttatgcaattattgaaggcctccctaacggggggcctttttttgtttctggtcacccgcttaacgatcgttggctgagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccattcccgagctgtcaccggatgtgctttccggtctgatgagtccgtgaggacgaaacagcctctacaaataattttgtttaatactagagaaagaggagaaatactagatgcgtgcactggttgttattcgtctgagccgtgttaccgatgcaaccaccagtccggaacgtcagctggaaagctgtcagcagctgtgtgcacagcgtggttgggatgttgttggtgttgccgaagatctggatgttagcggtgcagttgatccgtttgatcgtaaacgtcgtccgaatctggcacgttggctggcatttgaagaacagccgtttgatgttattgttgcctatcgtgttgatcgtctgacccgtagcattcgtcatctgcagcagctggttcattgggcagaagatcataaaaagctggttgtgagcgcaaccgaagcacattttgataccaccacaccgtttgcagcagttgttattgcactgatgggcaccgttgcacagatggaactggaagcaattaaagaacgtaatcgtagcgcagcccattttaacattcgtgcaggtaaatatcgtggtagcctgcctccgtggggttatctgccgacacgtgttgatggtgaatggcgtctggttccggatcctgttcagcgtgaacgtattctggaagtttatcatcgtgtggtggataatcatgaaccgctgcatctggttgcacatgatctgaatcgtcgtggtgttctgagcccgaaagattattttgcacagctgcagggtcgtgaaccgcagggtcgcgaatggtcagcaaccgcactgaaacgtagcatgattagcgaagcaatgctgggttatgcaaccctgaatggtaaaaccgttcgtgatgatgatggtgcaccgctggttcgtgcagaaccgattctgacacgtgaacagctggaagcactgcgtgccgaactggttaaaaccagccgtgcaaaaccggcagttagcaccccgagcctgctgctgcgtgttctgttttgtgcagtttgtggtgaaccggcatacaaatttgccggtggtggtcgtaaacatccgcgttatcgttgtcgtagcatgggttttccgaaacattgtggtaatggtacagttgcaatggcagaatgggatgcattttgcgaagaacaggttctggatctgctgggtgatgccgaacgtctggaaaaagtttgggttgcaggtagcgatagcgcagttgaactggccgaagttaatgcagaactggttgatctgaccagcctgattggtagtccggcatatcgtgccggtagtccgcagcgtgaagcactggatgcacgtattgcagcactggcagcacgtcaagaagaattagaaggtctggaagcccgtccgagcggttgggaatggcgtgaaaccggtcagcgttttggtgattggtggcgtgagcaggataccgcagccaaaaatacctggctgcgtagtatgaatgttcgcctgacctttgatgttcgcggtggtctgacgcgtaccattgattttggcgatctgcaagaatatgaacagcatctgcgtctgggtagcgttgttgaacgtctgcataccggcatgagctaactcggtaccaaattccagaaaagaggcctcccgaaaggggggccttttttcgttttggtccaatggcggcgcgccatcgaatggtgcaaaacctttcgcggtatggcatgatagcgcccggaagagagtcaattcagggtggtgaatatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacttttttgggcaaatcattaacgccgggcaaggggaagggcgctattcggagctgctggcgataaatctgcttgagcaattgttactgcggcgcatgctagcgattaacggatcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggtttggaagaaaaagtgaatgatgtagccgtcaagttgtcatgataagatcctattccagcgggattaaagaggagcgattaagcatggttactatcaatacggaatctgctttaacgccacgttctttgcgggatacgcggcgtatgaatatgtttgtttcggtagctgctgcggtcgcaggattgttatttggtcttgatatcggcgtaatcgccggagcgttgccgttcattaccgatcactttgtgctgaccagtcgtttgcaggaatgggtggttagtagcatgatgctcggtgcagcaattggtgcgctgtttaatggttggctgtcgttccgcctggggcgtaaatacagcctgatggcgggggccatcctgtttgtactcggttctatagggtccgcttttgcgaccagcgtagagatgttaatcgccgctcgtgtggtgctgggcattgctgtcgggatcgcgtcttacaccgctcctctgtatctttctgaaatggcaagtgaaaacgttcgcggtaagatgatcagtatgtaccagttgatggtcacactcggcatcgtgctggcgtttttatccgatacagcgttcagttatagcggtaactggcgcgcaatgttgggggttcttgctttaccagcagttctgctgattattctggtagtatttctgccaaatagcccgcgctggctggcggaaaaggggcgtcatattgaggcggaagaagtattgcgtatgctgcgcgatacgtcggaaaaagcgcgagaagaactcaacgaaattcgtgaaagcctgaagttaaaacagggcggttgggcactgtttaagatcaaccgtaacgtccgtcgtgctgtgtttctcggtatgttgttgcaggcgatgcagcagtttaccggtatgaacatcatcatgtactacgcgccgcgtatcttcaaaatggcgggctttacgaccacagaacaacagatgattgcgactctggtcgtagggctgacctttatgttcgccacctttattgcggtgtttacggtagataaagcagggcgtaaaccggctctgaaaattggtttcagcgtgatggcgttaggcactctggtgctgggctattgcctgatgcagtttgataacggtacggcttccagtggcttgtcctggctctctgttggcatgacgatgatgtgtattgccggttatgcgatgagcgccgcgccagtggtgtggatcctgtgctctgaaattcagccgctgaaatgccgcgatttcggtattacctgttcgaccaccacgaactgggtgtcgaatatgattatcggcgcgaccttcctgacactgcttgatagcattggcgctgccggtacgttctggctctacactgcgctgaacattgcgtttgtgggcattactttctggctcattccggaaaccaaaaatgtcacgctggaacatatcgaacgcaaactgatggcaggcgagaagttgagaaatatcggcgtctgataaggatcctaattggtaacgaatcagacaattgacggctcgagggagtagcatagggtttgcagaatccctgcttcgtccatttgacaggcacattatgcatcgatgataagctgtcaaacatgagcagatcctctacgccggacgcatcgtggccggcatcaccggcgccacaggtgcggttgctggcgcctatatcgccgacatcaccgatggggaagatcgggctcgccacttcgggctcatgagcaaatattttatctgaggtgcttcctcgctcactgactcgctgcacgaggcagacctcaggatctccaggcatcaaataaaacgaaaggctcagtcgaaagactgggcctttcgttttatctgttgtttgtcggtgaacgctctctactagagtcacactggctcaccttcgggtgggcctttctgcgtttataggatccgaagttcctattctctagaaagtataggaacttcgggtccccaataattacgatttaaattggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagaagttcctattctctagaaagtataggaacttcgcagacaaaaaaaatggcgcacaatgtgcgccatttttcacttcacaggtactattgttttgaattgaaaagggcgcttcggcgccctttttgcatttgttgacggcatatatttgtatatcgaagcgccctgatgggcgctttttttatttaatcgataaccagaacattgctggccgatgagctgaccattgggcctatccgggctgtcccgatggatattacgccgaagtatgtgggaattgccagcggattgatgaacgccgtgagaccaagcttacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 7 - DNA, 13822 bp, synthetic construct
  source 1..13822: mol_type = other DNA
cgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccaacgatctgttgatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattagccatgggcccgtgcgaatcactatatcagaccgctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggagtatcacgaggcagaatttcagataaaaaaaatccttagctttcgctaaggatgatttctggaattcgcggccgcttctagagtccctatcagtgatagagattgacatccctatcagtgatagagatactgagcactactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagtaggatcctaactcgacgtgcaggcttcctcgctcactgactcgctgcgctcggtcgttcggctgcggcgagcggtatcagctcactcaaaggcggtaatggcttgtcgacgacggcggactccgtcgtcaggatcatagggcgctgttatctgataaggcttatctggtctcattttctcttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggactaagcttccactggattgtacaattacataaaataaaatatctttattttcattacatctgtgtgttggttttttgtgtgcgctacttttttgagaccggtttgtctggtcaaccaccgcggactcagtggtgtacggtacaaaccccatccagctgatatccttgacagctagctcagtcctaggtataatgctagcggatctcgtcagtaacgagccctgggagaaaacaaaatgagccatctggcagaactggttgcaagcgcaaaagcagcaattagccaggcaagtgatgttgcagcactggataatgttcgtgttgaatatctgggtaaaaagggtcatctgaccctgcagatgaccacactgcgtgaactgcctccggaagaacgtccggcagccggtgcagttattaatgaagcaaaagaacaggttcagcaggcactgaatgcacgtaaagccgaactggaaagcgcagccctgaatgcccgtctggcagcagaaaccattgatgttagcctgcctggtcgtcgtattgaaaatggtggtctgcatccggttacacgtaccattgatcgcattgaaagctttttcggtgaactgggttttaccgttgcaaccggtccggaaattgaagatgattatcataatttcgatgccctgaacattccgggtcatcatccggcacgtgcagatcatgataccttttggtttgataccacacgtctgctgcgtacccagaccagcggtgttcagattcgtaccatgaaagcacagcagcctccgattcgtattattgcaccgggtcgtgtttatcgcaatgattatgatcagacccatacgccgatgtttcatcagatggaaggtctgattgtggataccaatattagcttcaccaatctgaaaggcaccctgcatgattttctgcgcaatttctttgaagaggacctgcagattcgttttcgtccgagctattttccgtttagcgaaccgagtgccgaagttgatgttatgggcaaaaatggtaaatggctggaagttttaggttgcggtatggttcatccgaatgttctgcgtaatgttggtattgatccggaagtttatagcggttttggttttggtatgggcatggaacgtctgaccatgctgcgttatggtgttaccgatctgcgtagcttctttgaaaatgatctgcgttttctgaaacagtttaaatgaggatctccaggcatcaaataaaacgaaaggctcagtcgaaagactgggcctttcgttttatctgttgtttgtcggtgaacgctctctactagagtcacactggctcaccttcgggtgggcctttctgcgtttatagcctatagtgagtcgtattacatggtcatagctgtttcctggcagctctggcccgtgtctcaaaatctctgatgttacattgcacaagataaaaatatatcatcatgcctcctctagaccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcat

SEQ ID NO: 8 - DNA, 8746 bp, synthetic construct
  source 1..8746: mol_type = other DNA
ctcttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgggctttgtgcgacagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcagagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccggatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctagatcccagggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatttggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcggtacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccgaatcctgcactacagcgacccctttatcctcttcctgttcctgctggccttcagcaccgctacaatcatgctgtgttttctgctgtccaccttcttcagcaaggcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcagatgatgctgctggacgccgccgtgtatggactgctggcttggtatctggaccaggtgttcccaggcgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagattttcgagccctgcggcagacctgccgtggacagactgaacatcaccttctacgagaaccagattaccgcctttctgggccacaacggcgctggcaagacaaccacattgagcatcctcacaggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgttggaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagatcaggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaagaactgatttttctgctcccgaacaagaacttcaagcaccgggcctacgccagcctgttcagagagctggaagaaaccctggccgacctgggcctgtctagctttggcatcagcgacacccctctcgaagagattttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcagaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggcagaggcctgatcgacctggctctgtctcaggccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatttaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggacccccaggctagaagaatgctctggaacgtgatcgtgtctatcatccgcgagggcagagctgtggtgctgacctctcacagcatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctagagccgctggtgcctctagacaagctcaggactaagcttccactggattgtacaattacataaaataaaatatctttattttcattacatctgtgtgttggttttttgtgtgcgctacttttttgagaccggtttgtctggtcaaccaccgcggactccgtcgtcaggatcatagggcgctgttatctgataaggcttatctggtctcatttt

SEQ ID NO: 9 - DNA, 48 bp, synthetic construct
  source 1..48: mol_type = other DNA
ggtttgtctggtcaaccaccgcggactcagtggtgtacggtacaaacc

SEQ ID NO: 10 - DNA, 38 bp, synthetic construct
  source 1..38: mol_type = other DNA
ggcttgtcgacgacggcggactccgtcgtcaggatcat

SEQ ID NO: 11 - DNA, 5852 bp, synthetic construct
  source 1..5852: mol_type = other DNA
caattattgaaggcctccctaacggggggcctttttttgtttctggtcacccgcttaacgatcgttggctgaacaaacagacaatctggtctgtttgtattatggaaaatttttctgtataatagattcaacaaacagacaatctggtctgtttgtattatagctgtcaccggatgtgctttccggtctgatgagtccgtgaggacgaaacagcctctacaaataattttgtttaatactagagaaagaggagaaatactagatgcgtgcactggttgttattcgtctgagccgtgttaccgatgcaaccaccagtccggaacgtcagctggaaagctgtcagcagctgtgtgcacagcgtggttgggatgttgttggtgttgccgaagatctggatgttagcggtgcagttgatccgtttgatcgtaaacgtcgtccgaatctggcacgttggctggcatttgaagaacagccgtttgatgttattgttgcctatcgtgttgatcgtctgacccgtagcattcgtcatctgcagcagctggttcattgggcagaagatcataaaaagctggttgtgagcgcaaccgaagcacattttgataccaccacaccgtttgcagcagttgttattgcactgatgggcaccgttgcacagatggaactggaagcaattaaagaacgtaatcgtagcgcagcccattttaacattcgtgcaggtaaatatcgtggtagcctgcctccgtggggttatctgccgacacgtgttgatggtgaatggcgtctggttccggatcctgttcagcgtgaacgtattctggaagtttatcatcgtgtggtggataatcatgaaccgctgcatctggttgcacatgatctgaatcgtcgtggtgttctgagcccgaaagattattttgcacagctgcagggtcgtgaaccgcagggtcgcgaatggtcagcaaccgcactgaaacgtagcatgattagcgaagcaatgctgggttatgcaaccctgaatggtaaaaccgttcgtgatgatgatggtgcaccgctggttcgtgcagaaccgattctgacacgtgaacagctggaagcactgcgtgccgaactggttaaaaccagccgtgcaaaaccggcagttagcaccccgagcctgctgctgcgtgttctgttttgtgcagtttgtggtgaaccggcatacaaatttgccggtggtggtcgtaaacatccgcgttatcgttgtcgtagcatgggttttccgaaacattgtggtaatggtacagttgcaatggcagaatgggatgcattttgcgaagaacaggttctggatctgctgggtgatgccgaacgtctggaaaaagtttgggttgcaggtagcgatagcgcagttgaactggccgaagttaatgcagaactggttgatctgaccagcctgattggtagtccggcatatcgtgccggtagtccgcagcgtgaagcactggatgcacgtattgcagcactggcagcacgtcaagaagaattagaaggtctggaagcccgtccgagcggttgggaatggcgtgaaaccggtcagcgttttggtgattggtggcgtgagcaggataccgcagccaaaaatacctggctgcgtagtatgaatgttcgcctgacctttgatgttcgcggtggtctgacgcgtaccattgattttggcgatctgcaagaatatgaacagcatctgcgtctgggtagcgttgttgaacgtctgcataccggcatgagctaactcggtaccaaattccagaaaagaggcctcccgaaaggggggccttttttcgttttggtccaatggcggcgcgccatcgaatggtgcaaaacctttcgcggtatggcatgatagcgcccggaagagagtcaattcagggtggtgaatatgagcccgaaacgtcgtacccaggcagaacgtgcaatggaaacccagggtaaactgattgcagcagcactgggtgttctgcgtgaaaaaggttatgcaggttttcgtattgcagatgttccgggtgcagccggtgttagccgtggtgcacagagccatcattttccgaccaaactggaactgctgctggcaacctttgaatggctgtatgagcagattaccgaacgtagccgtgcacgtctggcaaaactgaaaccggaagatgatgttattcagcagatgctggatgatgcagcagaattttttctggatgatgattttagcatcggcctggatctgattgttgcagcagatcgtgatccggcactgcgtgaaggtattcagcgtaccgttgaacgtaatcgttttgttgttgaagatatgtggctgggtgtgctggtgagccgtggtctgagccgtgatgatgccgaagatattctgtggctgatttttaacagcgttcgtggtctggtagttcgtagcctgtggcagaaagataaagaacgttttgaacgtgtgcgtaatagcaccctggaaattgcacgtgaacgttatgcaaaattcaaacgttgataaggatcctaattggtaacgaatcagacaattgacggctcgagggagtagcatagggtttgcagaatccctgcttcgtccatttgacaggcacattatgcatcgatgataagctgtcaaacatgagcagatcctctacgccggacgcatcgtggccggcatcaccggcgccacaggtgcggttgctggcgcctatatcgccgacatcaccgatggggaagatcgggctcgccacttcgggctcatgagcaaatattttatctgaggtgcttcctcgctcactgactcgctgcacgaggcagacctcaggatctccaggcatcaaataaaacgaaaggctcagtcgaaagactgggcctttcgttttatctgttgtttgtcggtgaacgctctctactagagtcacactggctcaccttcgggtgggcctttctgcgtttataggatctgcgaagttcctatactttctagagaataggaacttcggatctgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatca

SEQ ID NO: 12 - DNA, 40 bp, synthetic construct
  source 1..40: mol_type = other DNA
aaaatgagaccagataagccttatcagataacagcgccct

SEQ ID NO: 13 - DNA, 36 bp, synthetic construct
  source 1..36: mol_type = other DNA
tgagaccagataagccttatcagataacagcgccct

SEQ ID NO: 14 - DNA, 33 bp, synthetic construct
  source 1..33: mol_type = other DNA
tgagaccagataagccttatcagataacagcgc

(19) United States
(12) Patent Application Publication
(10) Pub. No.: US 2025/0066809 A1
(43) Pub. Date: Feb. 27, 2025

(54) PRODUCTION OF GENE THERAPY VECTOR IN ENGINEERED BACTERIA

(71) Applicant: ALDEVRON, LLC, Fargo, ND (US)

(72) Inventors: Jin HUH, Cambridge, MA (US); Jodi KENNEDY, Cambridge, MA (US)

(21) Appl. No.: 18/721,342

(22) PCT Filed: Dec. 20, 2022
(86) PCT No.: PCT/US2022/082078

§ 371 Date: Jun. 18, 2024

Related U.S. Application Data
(60) Provisional application No. 63/291,871, filed on Dec. 20, 2021.

(51) Int. Cl. C12N 15/85; C12N 9/12; C12P 19/34

(52) U.S. Cl. CPC C12N 15/85; C12N 9/1247; C12P 19/34; C12Y 207/07006; C12N 2800/107; C12N 2830/002

(57) ABSTRACT

Provided herein are improved methods of engineering bacterial cells for production of circular DNA vectors, circular DNA vectors produced by such methods, and pharmaceutical compositions containing such circular DNA vectors. Methods provided herein are amenable to large scale production of high-purity compositions of circular DNA vectors.

### CROSS-REFERENCE

This application claims the benefit of U.S. Provisional Application No. 63/291,871, filed on Dec. 20, 2021, which is incorporated herein by reference in its entirety.

### FIELD OF THE INVENTION

In general, the present disclosure involves therapeutic vectors in engineered bacteria.

### BACKGROUND

Gene therapy is emerging as a promising approach to treat a wide variety of diseases and disorders in human patients. Recombinant adeno-associated viral (rAAV) vectors have an established record of high-efficiency gene transfer in human patients and a variety of model systems. Genomes of rAAV vectors are advantageous for their ability to persist in vivo as circular episomes for the life of the target cell. On the other hand, rAAV-based vectors suffer substantial drawbacks, such as limited maximum payload, immunogenicity, and manufacturing inefficiencies.

To address some of these challenges in rAAV technology, non-viral alternatives have gained traction in recent years. However, development of a scalable non-viral gene therapy platform that enjoys the efficiency and persistence of rAAV has proven elusive. For example, traditional bacterial plasmid DNA vectors represent a versatile tool in gene delivery but are limited by having an abundance of bacterial components of plasmid DNA vectors, such as antibiotic resistance genes and transcriptional regulatory elements, which can lead to immunogenicity and loss of gene expression by transcriptional silencing.

Despite various efforts to improve plasmid DNA vectors by removing backbone components, there exists a need for non-viral vectors that have minimal bacterial elements and methods of producing them efficiently at large scale.

### SUMMARY

Disclosed herein are bacterially produced circular DNA vectors (e.g., therapeutic circular DNA vectors) and bacterial cells (e.g., engineered bacterial cells) that can be used to produce them from a parental plasmid, e.g., a parental plasmid inside the bacterial cells (e.g., engineered bacterial cells). Therapeutic circular DNA vectors provided herein contain small (e.g., less than 50-base pair) replication origins and lack selection markers (e.g., antibiotic resistance genes), which can reduce risks introduced by foreign sequences in the vector. Such bacterially produced circular DNA vectors can thereby be produced efficiently and at large scale for therapeutic applications.

Embodiments disclosed herein include an engineered bacterial cell comprising: (a) a Rep gene encoding a bacterial replication protein integrated into the bacterial genome; (b) a circular DNA vector comprising: (i) a coding sequence; and (ii) a replication origin that is dependent on the replication protein; wherein the circular DNA vector does not comprise a selectable marker. In some embodiments, the replication origin is less than 50 base pairs in length. In some embodiments, the replication origin and replication protein are from a ColE2-related plasmid. In some embodiments, the ColE2-related plasmid is ColE2-P9.

In some embodiments, the Rep gene is operatively coupled to a first inducible promoter. In some embodiments, the first inducible promoter is a T7 RNA polymerase-dependent promoter. In some embodiments, the engineered bacterial cell further comprises a gene encoding T7 RNA polymerase (T7RNAP) integrated into the bacterial genome. In some embodiments, the T7RNAP gene is operatively coupled to a second inducible promoter. In some embodiments, the second inducible promoter is Ptac.

In some embodiments, the engineered bacterial cell further comprises a gene encoding an exogenous restriction enzyme integrated into the bacterial genome. In some embodiments, the gene encoding the exogenous restriction enzyme is operatively coupled to a third inducible promoter. In some embodiments, the third inducible promoter is Pbad. In some embodiments, the bacterial genome does not comprise a recognition sequence for the exogenous restriction enzyme.

In some embodiments, the coding sequence encodes of the circular DNA vector comprises a therapeutic gene or nucleic acid. In some embodiments, the coding sequence is a eukaryotic sequence (e.g., a sequence expressible in a mammalian cell).

In some embodiments, the replication origin is the only bacterial sequence in the circular DNA vector.

In some embodiments, the engineered bacterial cell comprises at least 20 copies of the circular DNA vector. In some embodiments, the engineered bacterial cell is capable of maintaining the circular DNA vector through at least 20 rounds of cell division.

In some embodiments, the engineered bacterial cell does not comprise any extragenomic circular DNA molecules other than one or more copies of the circular DNA vector.

Embodiments disclosed herein include a culture comprising a plurality of any of the engineered bacterial cells described herein, wherein the mean numbers of copies of the circular DNA vector per engineered bacterial cell is at least 10. In some embodiments, the culture contains at least 10⁷ engineered bacterial cells.

Embodiments disclosed herein include an engineered bacterial cell comprising: (a) a Rep gene encoding a bacterial replication protein integrated into the bacterial genome; (b) a plasmid comprising: (i) a first segment comprising a coding sequence and a replication origin that is dependent on the bacterial replication protein, wherein the first segment does not comprise a selectable marker; and (ii) a second segment comprising a selectable marker; wherein the first segment is flanked by recognition sequences for at least one exogenous restriction enzyme or exogenous recombinase. In some embodiments, the recognition sequences flanking the first segment are the same. In some embodiments, the recognition sequences flanking the first segment are different. In some embodiments, the second segment further comprises a replication origin, wherein the replication origin in the second segment is orthologous to the replication origin in the first segment.

In some embodiments, the replication origin is less than 50 base pairs in length. In some embodiments, the replication origin and replication protein are from a ColE2-related plasmid. In some embodiments, the ColE2-related plasmid is ColE2-P9.

In some embodiments, the Rep gene is operatively coupled to a first inducible promoter. In some embodiments, the first inducible promoter is a T7 RNA polymerase-dependent promoter. In some embodiments, the engineered bacterial cell further comprises a gene encoding T7 RNA polymerase (T7RNAP) integrated into the bacterial genome. In some embodiments, the T7RNAP gene is operatively coupled to a second inducible promoter. In some embodiments, the second inducible promoter is Ptac.

In some embodiments, the engineered bacterial cell further comprises a gene encoding the exogenous restriction enzyme or exogenous recombinase integrated into the bacterial genome. In some embodiments, the gene encoding the exogenous restriction enzyme or exogenous recombinase is operatively coupled to a third inducible promoter. In some embodiments, the third inducible promoter is Pbad. In some embodiments, the bacterial genome does not comprise a recognition sequence for the exogenous restriction enzyme or the exogenous recombinase.

In some embodiments, the coding sequence of the first segment encodes a therapeutic gene or nucleic acid. In some embodiments, the coding sequence is a eukaryotic sequence (e.g., a sequence expressible in a mammalian cell).

In some embodiments, the engineered bacterial cell further comprises the exogenous restriction enzyme or exogenous recombinase.

Embodiments disclosed herein include a method of making a circular DNA vector, the method comprising: (a) contacting a plasmid within a bacterial cell with an exogenous restriction enzyme to excise a first segment from the plasmid, wherein the first segment is flanked by recognition sequences for the exogenous restriction enzyme, and wherein the first segment comprises a coding sequence and a replication origin dependent on a bacterial replication protein, thereby generating a linear DNA fragment comprising a 5′ end and a 3′ end with complimentary overhangs; and (b) ligating the 5′ and the 3′ end of the linear DNA fragment together to generate the circular DNA vector. In some embodiments, before step (a), the plasmid comprises a second segment comprising a selectable marker. In some embodiments, the second segment further comprises a replication origin, wherein the replication origin in the second segment is orthologous to the replication origin in the first segment. In some embodiments, the first segment does not comprise a selectable marker.

In some embodiments, contacting the plasmid within the cell with the exogenous restriction enzyme comprises inducing expression of the exogenous restriction enzyme within the cell. In some embodiments, a gene encoding the exogenous restriction enzyme is integrated into the bacterial genome operatively coupled to an inducible promoter. In some embodiments, the inducible promoter is Pad, and inducing expression of the exogenous restriction enzyme within the cell comprises providing arabinose to the cell. In some embodiments, contacting the plasmid within the cell with the exogenous restriction enzyme comprises introducing the exogenous restriction enzyme into the cell from outside of the bacterial cell.

In some embodiments, the ligating is performed by an exogenous ligase. In some embodiments, in the exogenous ligase is expressed from a gene integrated into the bacterial genome. In some embodiments, the exogenous ligase is introduced into the bacterial cell from outside the bacterial cell.

In some embodiments, the bacterial cell comprises a Rep gene encoding the bacterial replication protein integrated into the genome. In some embodiments, the Rep gene is operatively coupled to an inducible promoter capable of expressing the bacterial replication protein at a first expression level and a second expression level, wherein the first expression level is lower than the second expression level. In some embodiments, the first expression level of the bacterial replication protein causes the replication origin to be maintained at a first copy number, and the second expression level of the bacterial replication protein causes the replication origin to be maintained at a second copy number, wherein the first copy number is below 5, 10, 15, 20, or 50 copies per cell and the second copy number is at least 20, 50, 100, or 200 copies per cell. In some embodiments, the bacterial replication gene is expressed at the first expression level before step (b) and is not expressed at the second expression level before step (b). In some embodiments, the bacterial replication gene is expressed at the second expression level after step (b). In some embodiments, the inducible promoter is a PT7 dependent on T7 RNA polymerase. In some embodiments, the bacterial cell comprises a gene encoding T7 RNA polymerase integrated into the genome (T7RNAP). In some embodiments, the T7RNAP gene is operatively coupled to an inducible promoter. In some embodiments, the inducible promoter is P_{tac}. In some embodiments, the second segment of the plasmid further comprises a LacI gene encoding a lactose inhibitor protein capable of suppressing expression from the P_{tac} promoter, and wherein expression of the bacterial replication gene is maintained at or below the first expression level by expression of the lactose inhibitor protein. In some embodiments, after step (b) expression of the lactose inhibitor protein is reduced, thereby inducing the bacterial replication gene to be expressed at the second expression level and causing the circular DNA vector to be maintained at the second copy number.

In some embodiments, the method of making a circular DNA vector further comprises culturing the cell under conditions in which the selectable marker on the plasmid is not needed for continued growth, thereby generating a population of progeny of the bacterial cell that lack the selectable marker. In some embodiments, the population maintains the circular DNA vector after at least 50 doublings. In some embodiments, the population maintains the circular DNA vector after at least 100 doublings, e.g., at least 150 doublings, at least 200 doublings, at least 250 doublings, or at least 290 doublings. In some embodiments, the population maintains the circular DNA vector at an average copy number of at least 20 copies per cell after at least 50 doublings. In some embodiments, the population maintains the circular DNA vector at an average copy number of at least 20 copies per cell after at least 100 doublings. In some embodiments, the population maintains the circular DNA vector at an average copy number of at least 20 copies per cell after at least 150 doublings, at least 200 doublings, at least 250 doublings, or at least 290 doublings. In some embodiments, the method further comprises purifying the circular DNA vector.

In some embodiments, the replication origin is less than 50 base pairs in length (e.g., less than 45 base pairs in length, or about 40 base pairs in length). In some embodiments, the replication origin and replication protein are from a ColE2-related plasmid. In some embodiments, the ColE2-related plasmid is ColE2-P9. In some embodiments, the replication origin comprises, or consists of, SEQ ID NO: 2 (or reverse complement) or a functional variant thereof (e.g., a functional variant that has at least 90% sequence identity to SEQ ID NO: 2 (e.g., at least 92% sequence identity to SEQ ID NO: 2, at least 94% sequence identity to SEQ ID NO: 2, at least 95% sequence identity to SEQ ID) NO: 2, at least 96% sequence identity to SEQ ID NO: 2, at least 97% sequence identity to SEQ ID NO: 2, at least 98% sequence identity to SEQ ID NO: 2, at least 99 sequence identity to SEQ ID NO: 2, or 100% sequence identity to SEQ ID NO: 2)). In some embodiments, the replication origin comprises, or consists of, SEQ ID NO: 3 (or reverse complement) or a functional variant thereof (e.g., a functional variant that has at least 90% sequence identity to SEQ ID NO: 3 (e.g., at least 92% sequence identity to SEQ ID) NO: 3, at least 94% sequence identity to SEQ ID NO: 3, at least 95% sequence identity to SEQ ID NO: 3, at least 96% sequence identity to SEQ ID NO: 3, at least 97% sequence identity to SEQ ID NO: 3, at least 98% sequence identity to SEQ ID NO: 3, at least 99 sequence identity to SEQ ID NO: 3, or 100% sequence identity to SEQ ID NO: 3)). In some embodiments, the replication origin comprises, or consists of, SEQ ID NO: 4 (or reverse complement) or a functional variant thereof (e.g., a functional variant that has at least 90% sequence identity to SEQ ID NO: 4 (e.g., at least 92% sequence identity to SEQ ID NO: 4, at least 94% sequence identity to SEQ ID NO: 4, at least 95% sequence identity to SEQ ID NO: 4, at least 96% sequence identity to SEQ ID NO: 4, at least 97% sequence identity to SEQ ID NO: 4, at least 98% sequence identity to SEQ ID NO: 4, at least 99 sequence identity to SEQ ID NO: 4, or 100% sequence identity to SEQ ID NO: 4)).

Embodiments disclosed herein include a method of making a circular DNA vector, the method comprising: (a) obtaining any of the engineered bacterial cells described herein comprising a parental plasmid comprising a first segment comprising a coding sequence and a replication origin that is dependent on the bacterial replication protein, wherein the first segment does not comprise a selectable marker, and wherein the first segment is flanked by recognition sequences for at least one exogenous restriction enzyme; and (b) contacting the plasmid with an exogenous restriction enzyme to excise the first segment of the plasmid, thereby generating a linear DNA fragment flanked by complementary overhangs; and (c) self-ligating the linear DNA fragment to generate the circular DNA vector.

Embodiments disclosed herein include a method of making a circular DNA vector, the method comprising: (a) obtaining any of the engineered bacterial cells described herein comprising a parental plasmid comprising a first segment comprising a coding sequence and a replication origin that is dependent on the bacterial replication protein, wherein the first segment does not comprise a selectable marker, and wherein the first segment is flanked by recognition sequences for at least one exogenous recombinase; (b) contacting the plasmid with the exogenous recombinase that recognizes the recognition sequences flanking the first segment.

Embodiments disclosed herein include a pharmaceutical composition comprising (a) a circular DNA vector produced by any of the methods described herein; and (b) a suitable carrier for use in delivering the pharmaceutical composition to a subject.

In another aspect, provided is an engineered bacterial cell (e.g., E. coli) comprising a circular DNA vector comprising a coding sequence and a replication origin that is less than 50 base pairs in length, wherein the circular DNA vector lacks a selectable marker. In some embodiments, the engineered bacterial cell does not comprise any extragenomic DNA molecules other than one or more copies of the circular DNA vector. In some embodiments, the engineered bacterial cell does not comprise a gene encoding a selectable marker. In some embodiments, the engineered bacterial cell does not comprise a selectable marker on an extragenomic DNA molecule. In some embodiments, the replication origin is from a ColE2-related plasmid (e.g., a ColE2-P9 plasmid). In some embodiments, the engineered bacterial cell further includes a Rep gene encoding a bacterial replication protein that recognizes the origin of replication (e.g., a Rep gene is from a ColE2-P9 plasmid (e.g., SEQ ID NO: 1). In some embodiments, the Rep gene is integrated into the bacterial genome. In some embodiments, the Rep gene is operatively coupled to an inducible promoter.

In some embodiments, the circular DNA vector further comprises a recombination site (e.g., an attL, site (e.g., attL-GA) or an attR site).

In some embodiments, the circular DNA vector does not comprise any bacterial (or other prokaryotic or phage) sequence other than the origin of replication and, when present, the recombination site. In some embodiments, the origin of replication and recombination site together are no more than 90 base pairs in length.

In some embodiments, the engineered bacterial cell further includes a gene encoding a recombinase.

In some embodiments, the engineered bacterial cell comprises at least 10 copies of the circular DNA vector.

In another aspect, provided herein is an engineered bacterial cell comprising a plasmid that comprises: (a) a first segment comprising a coding sequence and a replication origin that is less than 50 base pairs in length, wherein the first segment does not comprise a selectable marker; and (b) a second segment comprising a selectable marker; wherein the first segment is flanked by recognition sequences for an exogenous recombinase. In some embodiments, the engineered bacterial cell further includes a gene encoding a Rep gene encoding a bacterial replication protein that recognizes the replication origin. In some embodiments, the Rep gene is integrated into the bacterial genome. In some embodiments, the Rep gene is operatively coupled to a first inducible promoter.

In some embodiments, the replication origin and replication protein are from a ColE2-related plasmid, e.g., ColE2-P9.

In some embodiments, the engineered bacterial cell further comprises a gene encoding the exogenous recombinase. In some embodiments, the gene encoding the exogenous recombinase is integrated into the bacterial genome. In some embodiments, the gene encoding the exogenous recombinase is on a plasmid or bacterial artificial chromosome. In some embodiments, the gene encoding the exogenous recombinase is operatively coupled to a second inducible promoter. In some embodiments, the second inducible promoter is a cuminic acid-inducible promoter.

In some embodiments, the recombinase is Bxb1. In some embodiments, the recognition sequences comprise attP-GA and attB-GA.

In another aspect, provided herein is a method of making a circular DNA vector, wherein the method includes inducing recombination of the plasmid in the engineered bacterial cell of any one of the previous aspects. In some embodiments, inducing recombination of the plasmid comprises inducing expression of the exogenous recombinase in the engineered bacterial cell.

In another aspect, provided is a method of producing a circular DNA vector, the method including inducing recombination of a plasmid in an engineered bacterial cell, wherein: (a) the plasmid comprises: (i) a first segment comprising a coding sequence and a replication origin that is less than 50 base pairs in length, wherein the first segment does not comprise a selectable marker, wherein the first segment is flanked by recognition sequences for an exogenous recombinase; and (ii) a second segment comprising a selectable marker; and (b) the engineered bacterial cell comprises a gene encoding the exogenous recombinase; wherein the inducing causes recombination of the plasmid, thereby producing the circular DNA vector comprising the first segment.

In some embodiments, the engineered bacterial cell further comprises a Rep gene encoding a bacterial replication protein that recognizes the origin of replication. In some embodiments, the Rep gene is integrated into the bacterial genome. In some embodiments, the Rep gene is operatively coupled to a first inducible promoter.

In some embodiments, the replication origin is a ColE2-P9 replication origin and/or the Rep gene is a ColE2-P9 Rep gene.

In some embodiments, the exogenous recombinase is on a plasmid or bacterial artificial chromosome. In some embodiments, wherein the gene encoding the exogenous recombinase is operatively coupled to a second inducible promoter.

In some embodiments, the inducing recombination of the plasmid comprises inducing expression of the gene encoding the exogenous recombinase. In some embodiments, the inducing recombination of the plasmid comprises introducing the plasmid into the engineered bacterial cell, wherein the exogenous recombinase is expressed in the engineered bacterial cell at the time of the introducing. In some embodiments, the exogenous recombinase is expressed at a non-induced level at the time of the introducing.

In some embodiments, the exogenous recombinase is Bxb1 and the recognition sequences comprise attP-GA and attB-GA. In some embodiments, the gene encoding Bxb1 is operatively coupled to a cuminic acid-inducible promoter, the engineered bacterial cell is maintained in the absence of cuminic acid at the time of the introducing, and the Bxb1 is expressed at a non-induced level at the time of the introducing.

In another aspect, provided is a circular DNA vector (e.g., an engineered circular DNA vector) comprising (a) a eukaryotic promoter; (b) a eukaryotic coding sequence; and (c) a bacterial replication origin that is less than 50 bp in length, wherein the circular DNA vector lacks a selectable marker. In some embodiments, the 3′ end of the eukaryotic coding sequence is linked to the 5′ end of the promoter by a sequence comprising the bacterial origin of replication, wherein the sequence comprising the bacterial origin of replication is less than 100 bp in length. In some embodiments, the circular DNA vector is monomeric supercoiled.

In another aspect, provided is a pharmaceutical composition comprising a circular DNA vector comprising (a) a eukaryotic promoter; (b) a eukaryotic coding sequence; and (c) a bacterial replication origin that is less than 50 bp in length, wherein the circular DNA vector lacks a selectable marker. In some embodiments, the 3′ end of the eukaryotic coding sequence is linked to the 5′ end of the promoter by a sequence comprising the bacterial origin of replication, wherein the sequence comprising the bacterial origin of replication is less than 100 bp in length; and a suitable carrier for use in delivering the pharmaceutical composition to a subject.

In another aspect, provided is a host cell (e.g., a mammalian cell, e.g., a human cell) comprising a circular DNA vector comprising (a) a eukaryotic promoter; (b) a eukaryotic coding sequence; and (c) a bacterial replication origin that is less than 50 bp in length, wherein the circular DNA vector lacks a selectable marker. In some embodiments, the 3′ end of the eukaryotic coding sequence is linked to the 5′ end of the promoter by a sequence comprising the bacterial origin of replication, wherein the sequence comprising the bacterial origin of replication is less than 100 bp in length. In some embodiments, the host cell expresses a protein encoded by the coding sequence. In some embodiments, the host cell is isolated in vitro. In some embodiments, the circular DNA vector is transfected into the host cell by electroporation.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a table showing results from a stability study in which three ColE2-P9 replication origins were tested for their ability to confer stability to plasmids over E. coli expansion.

FIG. 2 illustrates an example of an assembly method for a parental plasmid that can be used in embodiments disclosed herein.

FIG. 3 illustrates an experimental procedure for production of a test circular DNA vector according to embodiments disclosed herein.

FIG. 4 shows the results of agarose gel electrophoresis of extrachromosomal DNA purified from engineered bacteria grown in rich media with either chloramphenicol (“Cm”; lanes 7-12) or no chloramphenicol (“No Cm”; Lanes 1-6). Lanes 2, 3, 5, 8, 9, and 11 show bands corresponding to a test circular DNA vector produced by recombination from a test parental plasmid. Lanes 1, 4, 7, and 10, show bands corresponding to a test parental plasmid.

FIG. 5 is a graph showing the percentage of sfGFP-positive cells in the indicated growth media with or without chloramphenicol (“Cm”). ZB=Zymo Broth; TB=Terrific Broth; SB=Super Broth; SOB=Super Optimal Broth; SOC=Super Optimal Broth with Catabolite Repression; LB=Luria Broth.

FIG. 6 is a schematic chart showing an exemplary process of producing a circular DNA vector of the invention using counterselection.

FIGS. 7A to 7F are schematic drawings showing contents of a bacterial artificial chromosome (BAC)-based method of expressing Bxb1 to produce circular DNA vector. FIG. 7A is a Rep gene integrated into the host genome. FIGS. 7B and 7C are two alternative BAC designs; the Bxb1 of FIG. 7B is driven by a cuminic acid inducible promoter, whereas the Bxb1 of FIG. 7C is driven by an arabinose inducible promoter. FIG. 7D is a template plasmid, which, upon recombination by Bxb1, becomes the circular DNA vector of FIG. 7E and the byproduct of FIG. 7F. Because the circular DNA vector contains the replication origin, and (optionally) the byproduct contains a PheS counterselection marker, the circular DNA vector becomes the dominant species as the host bacteria duplicate and expand.

FIGS. 8A and 8B are photographs showing fluorescence of clones at 24 and 72 hours, respectively, post-transformation using BAC 1696.

FIGS. 9A and 9B are photographs showing fluorescence of clones at 24 and 72 hours, respectively, post-transformation using BAC 1697.

FIG. 10 is a set of photographs showing fluorescence of clones 24 hours after contact with cuminic acid inducer.

FIG. 11 is a set of photographs showing fluorescence of clones 24 hours after contact with arabinose inducer.

FIG. 12 is a series of photographs showing fluorescence of re-streaked 1696 colonies incubated overnight on LB agar plates under various conditions.

FIG. 13 is a series of photographs showing fluorescence of re-streaked 1697 colonies incubated overnight on LB agar plates under various conditions.

FIG. 14 is photograph of a gel electrophoresis experiment showing presence of circular DNA vector in counterselected cultures for both 1696 and 1697. A digestion map showing theoretical bands is shown to the left of the photograph.

FIG. 15A is a plasmid map of an exemplary ABCA4 template plasmid.

FIG. 15B is a plasmid map of an ABCA4 circular DNA vector resulting from the template plasmid of FIG. 15A.

FIG. 16A is a theoretical gel map showing banding patterns for circular DNA construct digests described in Example 7. FIG. 16B is a photograph of a gel showing actual banding patterns corresponding to FIG. 16A.

FIG. 17A is a histogram showing long-read sequencing data from purified ABCA4 circular DNA vectors produced using a 2-hour Kan resistance incubation with template plasmid. Major peaks are BAC and dimeric circular DNA vector.

FIG. 17B is a histogram showing long-read sequencing data from purified ABCA4 circular DNA vectors produced using an overnight Kan resistance incubation with template plasmid. Major peaks are monomeric circular DNA vector and BAC.

FIG. 18 is a plasmid map showing components of a helper plasmid useful for expressing Bxb1 in a bacterial host.

FIG. 19 is a photograph showing green fluorescent colonies (circled) which contain circular DNA vector without backbone byproduct as a result of Bxb1 expression by the helper plasmid of FIG. 18.

FIG. 20 is a set of drawings depicting a process of integrating Bxb1 into the host cell genome.

FIG. 21 is a photograph of a gel showing two positive clones for Bxb1 integration. 1696 plasmid controls are shown in a triplicate at the bottom left.

FIG. 22 is a photograph of a western blot showing that HEK293T cells transfected with bacterially produced ABCA4 circular DNA vectors express ABCA4 protein.

### DETAILED DESCRIPTION

Provided herein are improved methods for producing circular DNA vectors from parental plasmids within engineered bacterial cells, and related compositions. In the past, there have been efforts to improve plasmid DNA vectors by removing backbone components. For example, minicircles are made in bacterial cells using recombination to remove the backbone from the plasmid, producing a minicircle vector and a circular backbone byproduct. Minicircles, however, are difficult to produce at large scale, because their isolation requires purification from the backbone byproduct, which has a similar structure. Alternative vector types, such as nanoplasmids, have been designed for easier purification through positive selection by including a replication origin and selectable marker in the vector. But such extraneous elements are relatively large—generally hundreds of base pairs in length—and are foreign to the patient.

Disclosed herein are bacterially produced circular DNA vectors (e.g., therapeutic circular DNA vectors) and bacterial cells (e.g., engineered bacterial cells) that can be used to produce them from a parental plasmid, e.g., a parental plasmid inside the bacterial cells (e.g., engineered bacterial cells). Therapeutic circular DNA vectors provided herein contain small (e.g., less than 50-base pair) replication origins and lack selection markers (e.g., antibiotic resistance genes), which can reduce risks introduced by foreign sequences in the vector. Such bacterially produced circular DNA vectors can thereby be produced efficiently and at large scale for therapeutic applications. Additionally, by eliminating or reducing bacterial plasmid DNA sequences, such as RNAPII arrest sites, transcriptional silencing of a circular DNA vector can be reduced or eliminated, resulting in persistence of the vector sequence in an individual. In particular embodiments of the present invention, immunogenic components (e.g., bacterial endotoxin, DNA, or RNA, or bacterial signatures, such as CpG motifs) are absent in the present circular DNA vectors or are present at very low levels suitable for pharmaceutical or laboratory applications; therefore, the risk of stimulating a host immune response is reduced relative to conventional DNA vectors, such as plasmid DNA vectors. These and other aspects of the disclosed embodiments are discussed in more detail below.

### I. Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure. Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. Certain specific details of this description are set forth in order to provide a thorough understanding of various embodiments. However, one skilled in the art will understand that the present disclosure may be practiced without these details. In other instances, well known structures have not been shown or described in detail to avoid unnecessarily obscuring descriptions of the embodiments.

As used herein, “inducible promoter” refers to a promoter whose expression can be turned on or increased in response to a stimulus. The stimulus may be, for example, the presence of a particular molecule or culture conditions. The stimulus may also be, for example, absence of a particular molecule or culture conditions. As used herein “inducible promoters” include promoters whose expression can be turned on or increased by removal of a condition, such as the presence of a particular molecule or other culture condition, that suppresses expression from the promoter. In some embodiments, the inducible promoter is a T7 RNA polymerase-dependent promoter, a P_{tac} promoter, a P_{bad} promoter, a PT7 promoter, or a combination thereof. In some embodiments, the inducible promoter is Integrated into a bacterial genome and is operatively linked to a gene such as, for example, a gene encoding a Rep protein, a restriction enzyme, or a recombination enzyme.

As used herein, “exogenous” refers to any material introduced from or produced outside an organism, cell, tissue or system. For example, in embodiments described herein in which an exogenous restriction enzyme is present in an engineered bacterial cell, the exogenous restriction enzyme is not one that would be present in the engineered bacterial cell without the exogenous restriction enzyme being introduced into the engineered bacterial cell from outside the engineered bacterial cell. An exogenous restriction enzyme can be introduced into an engineered bacterial cell by, for example, introducing a gene encoding the exogenous restriction enzyme into the bacterial cell or introducing a restriction enzyme into the cell across the cell membrane, such as by electroporation. Embodiments described herein also include, for example, exogenous ligases and recombinases.

As used herein, a “parental plasmid” is a plasmid that contains both a vector sequence (as defined below) and a “backbone sequence” (as defined below). Embodiments disclosed herein include methods of making a circular DNA vector that include removing the backbone sequence from the vector sequence.

As used herein, a “vector sequence” of a parental plasmid refers to a portion of plasmid DNA that includes an origin of replication and a coding sequence for a gene of interest. In some descriptions of embodiments herein, a vector sequence is referred to as a “first segment” of a plasmid.

As used herein, a “backbone sequence” of a parental plasmid refers to a portion of plasmid DNA outside the vector sequence that includes one or more selectable markers such as drug resistance genes or fragments thereof. In some descriptions of embodiments herein, a backbone sequence is referred to as a “second segment” of a plasmid.

As used herein a “replication protein” is a protein that is necessary for initiation of replication at an origin of replication sequence that corresponds to the replication protein. A particular origin of replication sequence corresponds to a given replication protein if the origin of replication depends on the replication protein for initiation of replication at the origin of replication sequence. As an example, the replication protein encoded by a ColE2-P9 plasmid corresponds with the ColE2-P9 ori sequence; i.e., the ColE2-P9 replication protein is necessary for initiation of DNA replication at a ColE2-P9 ori sequence.

As used herein, a “functional variant” of a nucleic acid sequence differs in at least one nucleic acid residue from the reference nucleic acid sequence, such as a naturally occurring nucleic acid sequence, wherein relevant functional activity of the variant is at least 90% of the level of relevant functional activity of the reference nucleic acid sequence (e.g., substantially similar to the relevant function of the reference nucleic acid sequence). In this context, the difference in at least one nucleic acid residue may consist, for example, in a mutation of an nucleic acid residue to another nucleic acid, a deletion or an insertion. A variant may encode a homolog, isoform, or transcript variant of a therapeutic protein or a fragment thereof encoded by the reference nucleic acid sequence, wherein the homolog, isoform or transcript variant is characterized by a degree of identity or homology, respectively, as defined herein.

In some instances, a functional variant of a polynucleotide or polypeptide includes at least one nucleic acid substitution (e.g., 1-100 nucleic acid or amino acid substitutions, 1-50 nucleic acid or amino acid substitutions, 1-20 nucleic acid or amino acid substitutions, 1-10 nucleic acid or amino acid substitutions, e.g., 1 nucleic acid or amino acid substitution, 2 nucleic acid or amino acid substitutions, 3 nucleic acid or amino acid substitutions, 4 nucleic acid or amino acid substitutions, 5 nucleic acid or amino acid substitutions, 6 nucleic acid or amino acid substitutions, 7 nucleic acid or amino acid substitutions, 8 nucleic acid or amino acid substitutions, 9 nucleic acid or amino acid substitutions, or 10 nucleic acid or amino acid substitutions). Nucleic acid substitutions that result in the expressed polypeptide having an exchanged in amino acids from the same class are referred to herein as conservative substitutions. In particular, these are amino acids having aliphatic side chains, positively or negatively charged side chains, aromatic groups in the side chains or amino acids, the side chains of which can form hydrogen bridges, e.g., side chains which have a hydroxyl function. By conservative substitution, e.g., an amino acid having a polar side chain may be replaced by another amino acid having a corresponding polar side chain, or, for example, an amino acid characterized by a hydrophobic side chain may be substituted by another amino acid having a corresponding hydrophobic side chain (e.g., serine (threonine) by threonine (serine) or leucine (isoleucine) by isoleucine (leucine)).

In order to determine the percentage to which two sequences (e.g., nucleic acid sequences, e.g., DNA or amino acid sequences) are identical, the sequences can be aligned in order to be subsequently compared to one another. For this purpose, gaps can be inserted into the sequence of the first sequence and the component at the corresponding position of the second sequence can be compared. If a position in the first sequence is occupied by the same component as is the case at a corresponding position in the second sequence, the two sequences are identical at this position. The percentage, to which two sequences are identical, is a function of the number of identical positions divided by the total number of positions. The percentage to which two sequences are identical can be determined using a mathematical algorithm. A preferred, but not limiting, example of a mathematical algorithm, which can be used is the algorithm of Karlin et al. (1993), PNAS USA, 90:5873-5877 or Altschul et al. (1997), Nucleic Acids Res., 25:3389-3402. Such an algorithm can be integrated, for example, in the BLAST program. Sequences which are identical to the sequences of the present invention to a certain extent can be identified by this program.

As used herein, the term “flank,” “flanking,” and “flanked” refer to a pair of regions or points on a nucleic acid molecule (e.g., a plasmid) that are outside a reference region of the nucleic acid molecule. In some embodiments, a pair of regions or points flanking a reference region on a nucleic acid are adjacent to (i.e., abut) the reference region (i.e., there are no intervening bases between the reference point and the flanking point). In other embodiments, a pair of regions or points on a nucleic acid molecule that flank a reference region are separated from the reference region by one or more intervening bases (e.g., up to 1,000 intervening bases). For example, a first and second restriction site are said to flank a given sequence if the first restriction site is 200 bases upstream of the sequence and the second restriction site is 100 bases downstream of the sequence. In some embodiments, all intervening sequences between a flanking region or point and a reference region are devoid of bacterial sequences. In such embodiments, there are no bacterial sequences other than an ori sequence in a circular DNA vector produced by self-ligating a vector sequence that was cut out of a parental plasmid at restriction sites or recombination sites flanking the vector sequence. For example, in such embodiments, an exogenous restriction enzyme that cuts sites flanking a vector sequence may produce a circular DNA vector having a sequence between the 5′ end and 3′ end of the therapeutic sequence; however, this region contains no bacterial sequences (e.g., drug-resistance genes). Such intervening sequences may be artifacts from sticky end ligation, e.g., corresponding to overhang bases generated by the exogenous restriction enzyme.

The term “ABCA4” refers to any native ABCA4 from any vertebrate source, including mammals such as primates (e.g., human and cynomolgus monkeys) and rodents (e.g., mice and rats), unless otherwise indicated, as well as functional variants (e.g., natural or synthetic variants), e.g., mutants, muteins, analogs, subunits, receptor complexes, isotypes, splice variants, and fragments thereof. Functional variants can be determined on the basis of known ABCA4 signaling. ABCA4 encompasses full-length, unprocessed ABCA4, as well as any form of ABCA4 that results from native processing in the cell. An exemplary human ABCA4 sequence is provided as NCBI Reference Sequence: NG_009073 or NM_000350.

The term “MYO7A” refers to any native MYO7A (also known as DFNB2, MYU7A, NSRD2, USH1B, DFNA11, or MYOVIIA) from any vertebrate source, including mammals such as primates (e.g., human and cynomolgus monkeys) and rodents (e.g., mice and rats), unless otherwise indicated, as well as functional variants (e.g., natural or synthetic variants), e.g., mutants, muteins, analogs, subunits, receptor complexes, isotypes, splice variants, and fragments thereof. Functional variants can be determined on the basis of known MYO7A signaling. MYO7A encompasses full-length, unprocessed MYO7A, as well as any form of MYO7A that results from native processing in the cell. An exemplary human MYO7A sequence is provided as National Center for Biotechnology Information (NCBI) Gene ID: 4647.

As used herein, the term “self-replicating RNA molecule” refers to a self-replicating genetic element comprising an RNA that replicates from one origin of replication.

As used herein, the term “operatively linked” or “operatively coupled” refers to an arrangement of elements, wherein the components so described are configured so as to perform their usual function. A nucleic acid is “operatively linked” or “operatively coupled” when it is placed into a functional relationship with another nucleic acid sequence. For example, a promoter is operatively linked to one or more heterologous genes if it affects the transcription of the one or more heterologous genes. Further, control elements operatively linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence, and the promoter sequence can still be considered “operatively linked” or “operatively coupled” to the coding sequence.

As used herein, a “vector” refers to a nucleic acid molecule capable of carrying a sequence of interest to which is it linked into a target cell in which the sequence of interest can then be transcribed, replicated, processed, and/or expressed in the target cell. After a target cell or host cell processes the sequence of interest of the vector, the sequence of interest is not considered a vector. One type of vector is a “plasmid”, which refers to a circular double stranded DNA loop capable of autonomous replication and containing a bacterial backbone including a bacterial origin of replication and a selectable marker, into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”).

As used herein, the terms “individual” and “subject” are used interchangeably and include any mammal in need of treatment or prophylaxis, e.g., by a circular DNA vector, or pharmaceutical composition thereof, described herein. In some embodiments, the individual or subject is a human. In other embodiments, the individual or subject is a non-human mammal (e.g., a non-human primate (e.g., a monkey), a mouse, a pig, a rabbit, a cat, or a dog). The individual or subject may be male or female.

As used herein, an “effective amount” or “effective dose” of a circular DNA vector, or pharmaceutical composition thereof, refers to an amount sufficient to achieve a desired biological, pharmacological, or therapeutic effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat a disease may slow or stop disease progression or increase partial or complete response, relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, circular DNA vectors of the invention are used to delay development of a disease or to slow the progression of a disease.

The terms “level of expression” or “expression level” are used interchangeably and generally refer to the amount of a polynucleotide or an amino acid product or protein in a biological sample (e.g., retina). “Expression” generally refers to the process by which gene-encoded information is converted into the structures present and operating in the cell. Therefore, according to the invention, “expression” may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, the term “expression persistence” refers to the duration of time during which a sequence of interest, or a functional portion thereof (e.g., one or more coding sequences of a circular DNA vector), is expressible in the cell in which it was transfected (“intra-cellular persistence”) or any progeny of the cell in which it was transfected (“trans-generational persistence”). A sequence of interest, such as a therapeutic sequence, or functional portion thereof, may be expressible if it is not silenced, e.g., by DNA methylation and/or histone methylation and compaction. Expression persistence can be assessed by detecting or quantifying (i) mRNA transcribed from the sequence in the target cell or progeny thereof (e.g., through qPCR, RNA-seq, or any other suitable method) and (ii) protein translated from the sequence in the target cell or progeny thereof (e.g., through Western blot, ELISA, or any other suitable method). In some instances, expression persistence is assessed by detecting or quantifying therapeutic DNA in the target cell or progeny thereof (e.g., the presence of circular DNA vector in the target cell, e.g., through episomal DNA copy number analysis) in conjunction with either or both of (i) mRNA transcribed from the therapeutic sequence in the target cell or progeny thereof and (ii) protein translated from the therapeutic sequence in the target cell or progeny thereof. Expression persistence of a sequence of interest, or a functional portion thereof, can be quantified relative to a reference vector, such as a control vector having one or more bacterial signatures not present in the vector of the invention (e.g., a plasmid), using any gene expression characterization method known in the art. Expression persistence can be quantified at any given time point following administration of the vector. For example, in some embodiments, expression of a circular DNA vector of the invention persists for at least two weeks after administration if it is detectable in the target cell, or progeny thereof, two weeks after administration of the circular DNA vector. In some embodiments, expression of a gene “persists” in a target cell if it is detectable in the target cell at one week, two weeks, three weeks, four weeks, six weeks, two months, three months, four months, five months, six months, seven months, eight months, nine months, ten months, eleven months, one year, or longer after administration. In some embodiments, expression of a sequence is said to persist for a given period after administration if any detectable fraction of the original expression level remains (e.g., at least 1%, at least 5%, at least 10%, at least 20%, at least 30%, at least 40%, at least 50%, at least 70%, or at least 100% of the original expression level) after the given period of time (e.g., one week, two weeks, three weeks, four weeks, six weeks, two months, three months, four months, five months, six months, seven months, eight months, nine months, ten months, eleven months, one year, or longer after administration).

As used herein, “intra-cellular persistence” refers to the duration of time during which a sequence, or a functional portion thereof (e.g., one or more coding sequences of a circular DNA vector), is expressible in the cell in which it was transfected (e.g., a target cell, such as a post-mitotic cell or a quiescent cell). Intra-cellular persistence can be assessed by detecting or quantifying (i) mRNA transcribed from the sequence in the target cell and (ii) protein translated from the sequence in the target cell. In some instances, intra-cellular persistence is assessed by detecting or quantifying DNA in the target cell (e.g., the presence of circular DNA vector in the target cell) in conjunction with either or both of (i) mRNA transcribed from the sequence in the target cell and (ii) protein translated from the sequence in the target cell. In some embodiments, the circular DNA vector of the invention exhibits improved intra-cellular persistence relative to a reference vector (e.g., a plasmid DNA vector).

As used herein, “trans-generational persistence” refers to the duration of time during which a sequence, or a functional portion thereof (e.g., one or more coding sequences of a DNA vector), is expressible in progeny of the cell in which the gene was transfected (e.g., progeny of the target cell, such as first-generation, second-generation, third-generation, or fourth-generation descendants of the cell in which the gene was transfected, e.g., through a circular DNA vector). Trans-generational persistence accounts for any dilution of a gene over cell divisions and may therefore be useful in measuring persistence of a vector in a dividing tissue over time. In some embodiments, the circular DNA vector of the invention exhibits improved trans-generational persistence relative to a reference vector (e.g., a plasmid DNA vector). Trans-generational persistence can be assessed by detecting or quantifying (i) mRNA transcribed from the vector sequence in progeny of the target cell and (ii) protein translated from the vector sequence in progeny of the target cell. In some instances, intra-cellular persistence is assessed by detecting or quantifying DNA in progeny of the target cell (e.g., the presence of circular DNA vector in progeny of the target cell) in conjunction with either or both of (i) mRNA transcribed from the sequence in progeny of the target cell and (ii) protein translated from the sequence in progeny of the target cell. In some embodiments, the circular DNA vector of the invention exhibits improved trans-generational persistence relative to a reference vector (e.g., a plasmid DNA vector).

As used herein, the term “copy number” of a DNA molecule refers to the average number of copies of the DNA molecule per cell in a given population of cells.

The term “pharmaceutically acceptable” means safe for administration to a mammal, such as a human. In some embodiments, a pharmaceutically acceptable composition is approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which a vector or composition of the invention is administered. Examples of suitable pharmaceutical carriers are described in “Remington's Pharmaceutical Sciences,” Mack Publishing Co., Easton, PA., 23rd edition, 2020.

As used herein, the term “about” refers to a value within +10% variability from the reference value, unless otherwise specified.

For any conflict in definitions between various sources or references, the definition provided herein shall control.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

### II. Methods of Producing Circular DNA Vectors in Bacteria

Embodiments disclosed herein include methods of producing circular DNA vectors in engineered bacterial cells. The engineered bacterial cells disclosed herein can be used to produce circular DNA vectors from a parental plasmid. In some embodiments, the engineered bacterial cell includes a Rep gene encoding a bacterial replication protein integrated into the bacterial genome and a parental plasmid. In some embodiments, the Rep gene is included on an extrachromosomal DNA molecule such as, for example, a plasmid (e.g., a helper plasmid) or a bacterial artificial chromosome (“BAC”). In some embodiments, the Rep gene is included on the parental plasmid. The parental plasmid comprises a vector sequence and a backbone sequence. The vector sequence includes an ori sequence corresponding to the Rep gene and does not include a selectable marker. The backbone sequence includes a selectable marker and does not include the ori sequence included in the vector sequence, but may, in some embodiments, include a different ori sequence. The parental plasmid also has restriction enzyme recognition sequences or site-specific recombination sequences flanking the vector sequence arranged so that the plasmid backbone sequence can be separated from the vector sequence inside the cell by restriction digestion or site-specific recombination. In the case of restriction digestion, the circular DNA vector is then formed by self-ligation of the vector sequence. In the case of site-specific recombination, the circular DNA vector is formed as recombination is completed. Expression of the rep protein after separation of the vector sequence and formation of the circular DNA vector can maintain the circular DNA vector at a high copy number, despite the circular DNA vector lacking a selectable marker. In contrast, maintenance of the plasmid backbone sequence in the engineered bacterial cell after separation can be avoided by changing the culture conditions to remove selective pressure for the selectable marker. Culturing of a population of bacterial cells with a high copy number of circular DNA vector under conditions in which the parental plasmid is not maintained can efficiently produce a high yield of highly pure circular DNA vector.

### 1. Engineered Bacterial Cells

Methods of producing circular DNA vectors disclosed herein include the use of engineered bacterial cells, which can include, for example engineered E. coli bacterial cells or other suitable bacteria. In some embodiments, the engineered bacterial cells include an exogenous Rep gene encoding a replication protein integrated into the bacterial genome and a parental plasmid having an ori sequence that corresponds with the Rep gene. In some embodiments, the Rep gene is not integrated into the bacterial genome, but is present on an extrachromosomal DNA molecule, such as a plasmid or BAC.

In some embodiments, the engineered bacterial cells have an exogenous Rep gene integrated into the bacterial genome. Any suitable chromosomal integration process can be used to incorporate the Rep gene into the bacterial genome, including integration cassettes and procedures that are well-known in the art. In some embodiments, the Rep gene encodes a ColE2-P9 replication protein or a related protein. In some exemplary embodiments, the Rep gene encodes a ColE2-P9 replication protein that has the amino acid sequence set forth in SEQ ID NO: 1. Other suitable replication proteins include replication proteins encoded by naturally-occurring plasmids, including, for example, those that are related to ColE2-P9 such as ColE3-CA38. The replication proteins can be used in embodiments described herein in conjunction with their corresponding origin of replication sequences.

The ori sequence included in the vector sequence of a parental plasmid is chosen so that it corresponds with the Rep gene that is integrated into the genome of the engineered bacterial cell or is otherwise present in the engineered bacterial cell, such as on a plasmid or BAC. In some exemplary embodiments, the ori comprises a nucleotide sequence as set forth in SEQ ID NO: 2. Thus, embodiments of the engineered bacterial cells disclosed herein include a functional pair of a replication protein and origin of replication sequence that allow for replication of the parental plasmid and/or circular DNA vector. In some embodiments, the ori sequence present in the vector sequence is the ColE2-P9 ori sequence or a functional fragment thereof. In some embodiments, the ori sequence present in the vector sequence is a functional fragment of the ColE2-P9 ori sequence that has the DNA sequence set forth in SEQ ID NO: 2. The 40 base pair functional fragment set forth in SEQ ID NO:2 is capable of supporting vector replication in a cell expressing the ColE2-P9. In some embodiments, a shorter or longer functional fragment may be used. In some embodiments, a 31 base pair fragment of ColE2-P9 can be used. Other suitable ori sequences include, without limitation, ori sequences and functional fragments thereof that correspond with suitable Rep proteins, such as, for example the ori sequence of ColE3-CA38. In some embodiments, the ori is no more than or is less than 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 150, 200, 250, 300, 350, or 400 nucleotides in length. In some embodiments, the ori sequence is a functional modified version of a naturally occurring ori, such as, for example, an ori sequence that has been modified to be shorter than a corresponding naturally occurring ori sequence, while still retaining the ability to support replication initiation. In some embodiments, the ori sequence is a naturally occurring ori sequence.

In some embodiments, the Rep gene is operatively linked to an inducible promoter. Suitable inducible promoters include without limitation, a P_{T7} promoter that is induced by T7 RNA polymerase, a heat inducible P_{L} promoter, a P_{tac} promoter that is suppressible by LacI (and therefore inducible by the absence or removal of LacI), a P_{bad} promoter that is inducible by arabinose. Other inducible promoters known in the art can also be used in embodiments disclosed herein including, for example, bacteriophage promoters (e.g. P_{L}s1con, T3, T7, SP6) and bacterial promoters (e.g. PmgrB, pLlacO), Ptre2, pLtetO, Plac/ara, Pm). Examples of bacterial promoters for use in accordance with the present disclosure include, without limitation, positively regulated E. coli promoters such as positively regulated σ⁷⁰ promoters (e.g., inducible pBad/araC promoter, Lux cassette right promoter, modified lambda Prm promoter, plac Or2-62 (positive), pBad/AraC with extra REN sites, pBad, P(Las) TetO, P(Las) CIO, P(Rhl), Pu, FecA, pRE, cadC, hns, pLas, pLux), os promoters (e.g., Pdps), σ³² promoters (e.g., heat shock) and σ⁵⁴ promoters (e.g., glnAp2); negatively regulated E. coli promoters such as negatively regulated σ⁷⁰ promoters (e.g., Promoter (PRM+), modified lambda Prm promoter, TetR-TetR-4C P(Las) TetO, P(Las) CIO, P(Lac) IQ, RecA_DlcxO_dLacO1, dapAp, FccA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, plac_lux, pTet_Lac, plac/Mnt, pTet/Mnt, LsrA/cI, pLux/cI, LacI, LacIQ, pLacIQ1, pLas/cl, pLas/Lux, pLux/Las, pRecA with LexA binding site, reverse bBa_R0011, pLacI/ara-1, pLacIq, rmB P1, cadC, hns, PfhuA, pBad/araC, nhaA, OmpF, RcnR), σ^{S} promoters (e.g., Lutz-Bujard LacO with alternative sigma factor σ³⁸), σ³² promoters (e.g., Lutz-Bujard LacO with alternative sigma factor σ³²), and σ⁵⁴ promoters (e.g., glnAp2); negatively regulated B. subtilis promoters such as repressible B. subtilis σ^{A} promoters (e.g., Gram-positive IPTG-inducible, Xyl, hyper-spank) and σ^{B} promoters. Other inducible bacterial promoters may be used in accordance with the present disclosure. For example, a cuminic acid inducible promoter, such as pCymRC, may be used in some embodiments.

In some embodiments, the expression level of the replication protein affects the copy number of a parental plasmid or circular DNA vector comprising a corresponding ori sequence. Thus, when a relatively low copy number (e.g., an average of less than 5, 10, or 20 copies per cell) is desired, the engineered bacterial cells can be maintained in conditions in which the replication protein is expressed at a relatively low level. When a relatively high copy number is desired (e.g., an average of more than 20, 50, or 100 copies per cell), the engineered bacterial cells can be maintained in conditions in which the replication protein is expressed from an inducible promoter at a relatively high level. In some embodiments, the Rep gene is operatively linked to an inducible promoter that provides a first level of expression in non-inducing conditions and that can be induced to provide a second, higher level of expression that results in a higher copy number of a parental plasmid or a circular DNA vector that comprises a corresponding ori sequence. In some embodiments, it is advantageous to maintain the parental plasmid in the engineered bacterial cell at a low copy number before the vector sequence and backbone sequence of the parental plasmid are separated. In embodiments in which the vector sequence and backbone sequence are separated by restriction digestion, having a relatively low copy number can help to ensure that the linearized vector sequence self-ligates rather than ligating with backbone sequence or other copies of the vector sequence. After separation of the vector sequence from the backbone sequence, and formation of a circular DNA vector, it is advantageous in some embodiments to have the circular DNA vector, which contains an ori sequence, to be maintained at a relatively high copy number in order to produce a high yield of circular DNA vector. Thus, in some embodiments, after formation of the circular DNA vector within the engineered bacterial cell, higher expression levels of the replication protein are induced by, for example, adding a molecule that induces higher expression from the inducible promoter operatively linked to the Rep gene. In some embodiments the inducible promoter is maintained in an uninduced state until after separation of the vector sequence from the backbone sequence. In some embodiments, the inducible promoter is induced after separation of the vector sequence from the backbone sequence. In some embodiments, the inducible promoter is induced simultaneous with the separation of the vector sequence from the backbone sequence. In some embodiments, the inducible promoter is induced before the separation of the vector sequence from the backbone sequence.

In some embodiments, the copy number of the parental plasmid is maintained at a copy number of at least about, at most about, or about 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, copies per cell, or between any two of these values. In some embodiments, the copy number of the circular DNA vector is maintained at at least about, at most about, or about 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, or 400 copies per cell, or between any two of these values.

In some embodiments, the Rep gene integrated into the engineered bacterial cell genome is under control of a constitutive or non-inducible promoter. In some embodiments, expression of the replication protein is not adjusted before, during, or after the separation of the vector sequence from the backbone sequence. In some embodiments, the parental plasmid before separation and the circular DNA vector after separation are maintained at a copy number of at least about, at most about, or about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 copies per cell, or between any two of these values.

In addition to a parental plasmid or a vector sequence separated from a parental plasmid, engineered bacteria may also include other extrachromosomal DNA molecules such as helper plasmids or BACs. The extrachromosomal DNA molecules may encode, for example, exogenous recombinases, restriction enzymes, replication proteins, ligases, selectable markers, counterselection markers, or reporter genes. In some embodiments, extrachromosomal DNA molecules other than the vector sequence are removed from the engineered bacterial cell before purification of a circular DNA vector from a culture of engineered bacterial cells. In some embodiments, an extrachromosomal DNA molecule can be removed from engineered bacterial cells by culturing cells under conditions that do not apply selective pressure for maintaining the extrachromosomal DNA molecule or by culturing cells under counterselection conditions that reduce or eliminate growth of cells that include the extrachromosomal DNA molecule.

In some embodiments, extrachromosomal DNA molecules included in engineered bacterial cells include reporter constructs that can be used to track the presence of the extrachromosomal DNA molecule in cells. For example, a backbone sequence of a parental plasmid or a helper plasmid or BAC can include genes encoding a visually detectable protein such as GFP or RFP. In that case, visual observation of colony color under UV light can reveal whether the extrachromosomal DNA molecule is present in cells of the colony. In this way, colonies that lack a given extrachromosomal DNA molecule can be detected. Other suitable reporter constructs that can be detected in other ways can also be used to determine whether an engineered bacterial cell or colony contains a given extrachromosomal DNA molecule.

### B. Parental Plasmid

Embodiments of engineered bacteria disclosed herein include a parental plasmid (also revered to as a template plasmid or plasmid template) that comprises a vector sequence and a backbone sequence separated from each other by two restriction sites or recombination sites.

The vector sequence includes an ori sequence and a sequence of interest, which in some embodiments is a therapeutic coding sequence, a reporter construct, or a combination thereof. The vector sequence can include any of the components of circular DNA vector embodiments described herein. In some embodiments, the vector sequence does not comprise any sequences of bacterial origin other than the ori sequence. In some embodiments, the parental plasmid includes restriction sites or recombination sites immediately adjacent to the ori sequence and/or a therapeutic sequence or reporter construct so that there is no extraneous or non-functional DNA included in the vector sequence that becomes the circular DNA vector.

In some embodiments, the backbone sequence includes a selectable marker and does not include an ori sequence corresponding to the exogenous replication protein encoded by an integrated Rep gene. In some embodiments, the backbone sequence comprises an ori sequence that does not correspond to the integrated Rep gene, i.e., that is orthologous to the ori sequence in the vector sequence and to the integrated Rep gene. In some embodiments, the selectable marker comprised in the backbone sequence helps ensure that the parental plasmid is maintained in a population of engineered bacterial cells cultured under conditions wherein the selectable marker is necessary for cell growth or survival. For example, in some embodiments, the selectable marker is an antibiotic resistance gene. Culturing engineered bacterial cells in the presence of the corresponding antibiotic applies selective pressure so that the parental plasmid is maintained in a population of engineered bacterial cells. However, upon removal of selective pressure, such as by changing the growth media to one that lacks the antibiotic corresponding to an antibiotic resistance gene, a DNA molecule that includes the antibiotic resistance gene may be lost from the population, especially if such DNA molecule does not comprise an ori sequence. Thus, after separation of the vector sequence from the backbone sequence, the backbone sequence may be lost from the population or fail to be maintained in significant quantities if culture conditions do not apply selective pressure to maintain it.

In some embodiments, the backbone sequence comprises a counterselection marker. The counterselection marker may provide a way to selectively grow cells that do not include the backbone sequence. In some embodiments, growing cells under counterselection conditions after separation of the vector sequence from the backbone sequence may promote purity and reduce the amount of the backbone sequence in the culture and/or in a composition comprising purified vector sequence. Suitable counterselection markers are known in the art and may include, for example, pheS, sacB, thyA, lacY, gata-1, ccdB, rpsL, or tetAR.

Restriction sites or recombination sites flanking the vector sequence in the parental plasmid can be selected from any suitable restriction sites or recombination sites that do not occur within the vector sequence.

### C. Restriction Digestion & Ligation

Embodiments disclosed herein include a step of restriction digestion to separate the vector sequence from the backbone sequence of the parental plasmid. In some embodiments, the restriction digestion occurs within the engineered bacterial cell. In some embodiments, the restriction enzyme that digests the parental plasmid is an exogenous restriction enzyme that is expressed from an exogenous restriction enzyme gene introduced into the engineered bacterial cell. In some embodiments, the exogenous gene is integrated into the genome of the engineered bacterial cell. In some embodiments, the exogenous gene is encoded on a plasmid or BAC within the engineered bacterial cell. In some embodiments, it is necessary to suppress or delay induction of expression of the restriction enzyme until such time as separation of the vector sequence from the backbone sequence of the parental plasmid is desired. Thus, in some embodiments, the exogenous gene is operatively linked to an inducible promoter. When separation is desired, expression of the restriction enzyme can be induced, and the separation can proceed.

In some embodiments, the restriction enzyme used to separate the vector sequence from the backbone sequence is an exogenous restriction enzyme that is introduced into the engineered bacterial cell across the cell membrane. In some embodiments, this is accomplished by electroporation. A non-limiting example of an electroporation and digestion procedure is as follows: Electrocompetent engineered E. coli harboring a parent plasmid are cultured to OD of 0.8 in SOB at 30° C. The bacteria are washed three times with ice cold 10% glycerol and resuspended in 10% glycerol. 0.5 μl of each restriction enzyme and ligase are mixed with the electrocompetent cells—1 μg of DNA is digested with 10 units of restriction enzymes. The mixture is transferred to a cuvette (1 mm gap) and electroporated using an electroporator (BTX) using the 1800 volt setting. The cells are rescued by growing in SOC for 1 hr at 37° C. and arc plated on LB agar plate without antibiotics. Colonies are grown and DNA is purified using QIAGEN miniprep kit.

After the separation of the vector sequence, the circular DNA vector can be formed by self-ligating the vector sequence. In some embodiments, the ligation occurs within the engineered bacterial cell. In some embodiments, the ligase that joins the ends of the vector sequence is an exogenous ligase that is expressed from an exogenous ligase gene introduced into the engineered bacterial cell. The ligase can be, for example, T3 ligase, a T4 ligase, or a T7 ligase. In some embodiments, the exogenous ligase gene is integrated into the genome of the engineered bacterial cell. In some embodiments, the exogenous ligase gene is encoded on a plasmid within the engineered bacterial cell. In some embodiments, expression of the ligase is suppressed or is not induced until such time as separation of the vector sequence from the backbone sequence of the parental plasmid is accomplished. Thus, in some embodiments, the exogenous ligase gene is operatively linked to an inducible promoter.

In some embodiments the ligase is an exogenous ligase that is introduced into the engineered bacterial cell across the cell membrane. In some embodiments, this is accomplished by electroporation, which may be done according to the electroporation protocol described above. In some embodiments, the electroporation of restriction enzymes and ligase is done in a single step with both restriction and ligase enzymes entering the cells in a single electroporation step. In some embodiments, the restriction enzyme(s) and ligase are added separately to the cells.

In some embodiments, self-ligation of the vector sequence is accomplished by an endogenous ligase produced by the engineered bacterial cell.

In some embodiments, an exogenous restriction enzyme and an exogenous ligase are present within the engineered bacterial cell at the same time. In some embodiments, the exogenous restriction enzyme is introduced into the engineered bacterial cell (e.g., by electroporation of an exogenous restriction enzyme, by transformation with a DNA molecule encoding an exogenous restriction enzyme, or by induction of expression of an exogenous restriction enzyme gene under control of an inducible promoter) before the exogenous ligase is introduced into the engineered bacterial cell (e.g., by electroporation of an exogenous ligase, by transformation with a DNA molecule encoding an exogenous ligase, or by induction of expression of an exogenous ligase gene under control of an inducible promoter). In some embodiments, the exogenous restriction enzyme is introduced into the engineered bacterial cell before the exogenous ligase is introduced into the engineered bacterial cell. In some embodiments, the exogenous restriction enzyme is introduced into the engineered bacterial cell at the same time as the exogenous ligase.

### D. Site Specific Recombination

Site-specific recombination may be carried out using various systems that lead to site-specific recombination between sequences. In some embodiments, the site-specific recombination involves two specific sequences that are capable of recombining with one another in the presence of a recombinase.

In some embodiments, the recombinase that separates the vector sequence from the plasmid sequence is an exogenous recombinase that is expressed from an exogenous recombinase gene introduced into the engineered bacterial cell. In some embodiments, the exogenous recombinase gene is integrated into the genome of the engineered bacterial cell. In some embodiments, the exogenous recombinase gene is encoded on a plasmid or BAC within the engineered bacterial cell. In some embodiments, it is necessary to suppress or delay induction of expression of the recombinase until such time as separation of the vector sequence from the backbone sequence of the parental plasmid is desired. Thus, in some embodiments, the exogenous recombinase gene is operatively linked to an inducible promoter, such as any of the inducible promoters disclosed herein. When separation is desired, expression of the exogenous recombinase can be induced, and the separation can proceed. In some embodiments, the exogenous recombinase is expressed at the time that the parental plasmid is introduced into the engineered bacterial cell, which may cause the parental plasmid to undergo recombination without having to induce expression of the recombinase. In some embodiments, the recombinase is expressed at a relatively low level at the time the parental plasmid is introduced into the engineered bacterial cell. As an example, in some embodiments, the engineered bacterial cell may include an exogenous recombinase gene (which may be, for example, integrated into the bacterial chromosome or included on a plasmid or BAC present within the bacterial cell before introduction of the parental plasmid into the engineered bacterial cell) that is operatively coupled to an inducible promoter that provides for a low level of expression in non-inducing conditions. Introducing the parental plasmid into an engineered bacterial cell with an appropriately low level of expression of the exogenous recombinase may allow for growth of colonies on media selective for the selectable marker on the backbone sequence of the parental plasmid, while also inducing sufficient recombination of the parental plasmid to generate a population of cells in the colony that have the vector sequence separated from the backbone sequence.

In some embodiments, the recombinase used to separate the vector sequence from the backbone sequence is an exogenous recombinase that is introduced into the engineered bacterial cell across the cell membrane. In some embodiments, this is accomplished by electroporation. A non-limiting example of an electroporation and recombination procedure is as follows: Electrocompetent engineered E. coli harboring a parent plasmid is cultured to OD of 0.8 in SOB at 30° C. The bacteria are washed three times with ice cold 10% glycerol and resuspended in 10% glycerol. 1 μl of Cre (15 units, NEB, M0298M) is mixed with 50 μl of electrocompetent cells. The mixture is transferred to a cuvette (1 mm gap) and electroporated using an electroporator (BTX) using the 1800 volt setting. The cells are rescued by growing in SOC for 1 hr at 37° C. and are plated on LB agar plate without antibiotics. Colonies are grown and DNA is purified using QIAGEN miniprep kit. 1 μg of DNA is digested with 10 units of restriction enzymes.

The specific recombination system used in embodiments disclosed herein can be of different origins. In particular, the specific sequences and the recombinases used can belong to different structural classes, such as the integrase family of bacteriophage A or to the resolvase family of the transposon Tn3.

Recombinases belonging to the integrase family of bacteriophage A include, for example, the integrase of the phages lambda (Landy et al., Science 197:1147, 1977), P22, and ¢80 (Leong et al., J. Biol. Chem. 260:4468, 1985), HP1 of Haemophilus influenza (Hauser et al., J. Biol. Chem. 267 6859, 1992), the Cre integrase of phage P1 (which recognizes and causes recombination at LoxP sites), the integrase of the plasmid pSAM2 (EP 350,341) or alternatively the FLP recombinase of the 2μ plasmid. In embodiments in which circular DNA vectors are prepared by recombination by means of a site-specific system of the integrase family of bacteriophage λ, the resulting circular DNA vectors generally comprise a sequence resulting from the recombination between two att attachment sequences of the corresponding bacteriophage or plasmid.

Recombinases belonging to the family of the transposon Tn3 include, for example, the resolvase of the transposon Tn3 or of the transposons Tn21 and TnS22 (Stark et al., Trends Genet, 8, 432-439, 1992); the Gin invertase of bacteriophage mu, or, alternatively, the resolvase of plasmids, such as that of the par fragment of RP4 (Albert et al., Mol. Microbiol. 12:131, 1994). In embodiments in which circular DNA vectors are prepared by recombination by means of a site-specific system of the family of the transposon Tn3, the resulting circular DNA vectors generally comprise a sequence resulting from the recombination between two recognition sequences of the resolvase of the transposon in question.

In some embodiments, site-specific recombination sequences on the parental plasmid are derived from a bacteriophage. In some embodiments, the sequences are attachment sequences (attP and attB sequences) of a bacteriophage integrase or sequences derived from such attachment sequences. These sequences are capable of recombining specifically with one another in the presence of a recombinase referred to as an integrase with or without an excisionase. The term “sequences derived from such attachment sequences” includes the sequences obtained by modification(s) of the attachment sequences of the bacteriophages that retain the capacity to recombine specifically in the presence of the appropriate recombinase. Thus, such sequences can be reduced fragments of these sequences or, alternatively, fragments extended by the addition of other sequences (restriction sites, and the like). They can also be variants obtained by mutations, in particular by point mutations, such as attP-GA and attB-GA attachment sequences, for example.

In some embodiments, the recognition sequences and recombinase used are from tyrosine recombinase family members such as, for example, Flp, XerC, XerD, λ integrase, or HP1 integrase, or serine recombinase family members such as, for example, ϕBT1, TP901, Bxb1, MR11, A118, ϕK38, ϕC31, or Wβ.

In some embodiments, the recognition sequences and recombinase are from Bxb1 (e.g., the exogenous recombinase is Bxb1 and the recognition sequences are attP-GA and attB-GA).

### E. Amplification of Circular DNA Vector by Culturing Cells

After formation of a circular DNA vector in engineered bacterial cells according to embodiments described herein, the amount of circular DNA vector produced can be increased by culturing a population of engineered bacterial cells comprising a circular DNA vector. The culture conditions can be chosen to maximize bacterial cell growth and production of additional copies of a circular DNA vector. In some embodiments, the culture conditions are chosen so as to induce a high level of expression of a replication protein and thereby support a high copy number of the circular DNA vector having the corresponding ori sequence. In some embodiments, the culture conditions are chosen to remove selective pressure for maintenance of the backbone sequence that comprises a selectable marker, such that the backbone sequence is not maintained through rounds of cell division. In some embodiments, culture conditions are chosen that provide counterselection pressure for a counterselection marker present on a backbone sequence so that cells that include the backbone sequence have diminished growth potential or cannot grow.

In some embodiments, culturing a population of engineered bacterial cells comprising the circular DNA vector results in maintenance of the circular DNA vector in such cultured cells through at least 2, 3, 4, 5, 6, 7, 8, 9, 10, 15, 20, 25, 30, 35, 40, 45, 50, 55, 60, 65, 70, 75, 80, 85, 90, 95, or 100 rounds of cell division. In some embodiments the cultured population of engineered bacterial cells maintains the circular DNA vector at an average copy number of at least 1, 2, 3, 4, 5, 6, 7, 8, 9, 10, 11, 12, 13, 14, 15, 16, 17, 18, 19, 20, copics per cell after at least 10, 11, 12, 13, 14, 15, 16, 17, 18, 19, 20, 25, 30, 35, 40, 45, 50, 55, 60, 65, 70, 75, 80, 85, 90, 95, or 100 doublings (e.g., at least 1 copy per cell after at least 10 doublings (e.g., at least 5 copies per cell after at least 10 doublings, at least 10 copies per cell after at least 10 doublings, or at least 20 copies per cell after at least 10 doublings), at least 1 copy per cell after at least 20 doublings (e.g., at least 5 copies per cell after at least 20 doublings, at least 10 copies per cell after at least 20 doublings, or at least 20 copies per cell after at least 20 doublings), at least 1 copy per cell after at least 50 doublings (e.g., at least 5 copies per cell after at least 50 doublings, at least 10 copies per cell after at least 50 doublings, or at least 20 copies per cell after at least 50 doublings), or at least 1 copy per cell after at least 100 doublings (e.g., at least 5 copies per cell after at least 100 doublings, at least 10 copies per cell after at least 100 doublings, or at least 20 copies per cell after at least 100 doublings)). In some embodiments, the average copy number of backbone sequence after separation of the vector sequence from the backbone sequence is less than 5, 4, 3, 2, 1, 0.5, 0.1, 0.01, or 0.001 copies per cell or is undetectable after at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 doublings (e.g., less than 0.001 copies per cell after at least 1 doubling (e.g., less than 0.001 copies per cell after at least 10 doublings, less than 0.001 copies per cell after at least 20 doublings, less than 0.001 copies per cell after at least 50 doublings, or less than 0.001 copies per cell after at least 100 doublings), less than 0.01 copies per cell after at least 1 doubling (e.g., less than 0.01 copies per cell after at least 10 doublings, less than 0.01 copies per cell after at least 20 doublings, less than 0.01 copies per cell after at least 50 doublings, or less than 0.01 copies per cell after at least 100 doublings), less than 0.1 copies per cell after at least 1 doubling (e.g., less than 0.1 copies per cell after at least 10 doublings, less than 0.1 copies per cell after at least 20 doublings, less than 0.1 copies per cell after at least 50 doublings, or less than 0.1 copies per cell after at least 100 doublings), or less than 1 copy per cell after at least 1 doubling (e.g., less than 1 copy per cell after at least 10 doublings, less than 1 copy per cell after at least 20 doublings, less than 1 copy per cell after at least 50 doublings, or less than 1 copy per cell after at least 100 doublings)). In some embodiments, the average copy number of backbone sequence after separation of the vector sequence from the backbone sequence is less than 5, 4, 3, 2, 1, 0.5, 0.1, 0.01, or 0.001 copies per cell or is undetectable after at most 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 doublings (e.g., less than 0.001 copies per cell after at most 1 doubling (e.g., less than 0.001 copies per cell after at most 10 doublings, less than 0.001 copies per cell after at most 20 doublings, less than 0.001 copies per cell after at most 50 doublings, or less than 0.001 copies per cell after at most 100 doublings), less than 0.01 copies per cell after at most 1 doubling (e.g., less than 0.01 copies per cell after at most 10 doublings, less than 0.01 copies per cell after at most 20 doublings, less than 0.01 copies per cell after at most 50 doublings, or less than 0.01 copies per cell after at most 100 doublings), less than 0.1 copies per cell after at most 1 doubling (e.g., less than 0.1 copies per cell after at most 10 doublings, less than 0.1 copies per cell after at most 20 doublings, less than 0.1 copies per cell after at most 50 doublings, or less than 0.1 copies per cell after at most 100 doublings), or less than 1 copy per cell after at most 1 doubling (e.g., less than 1 copy per cell after at le most at 10 doublings, less than 1 copy per cell after at most 20 doublings, less than 1 copy per cell after at most 50 doublings, or less than 1 copy per cell after at most 100 doublings)).

In some embodiments, culturing a population of engineered bacterial cells comprising the circular DNA vector results in maintenance of the circular DNA vector in such cultured cells through at least 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 290, 294, 300, 350, 400, 450, or 500 rounds of cell division (e.g., as confirmed by Sanger sequencing). In some embodiments the cultured population of engineered bacterial cells maintains the circular DNA vector at an average copy number of at least 1, 2, 3, 4, 5, 6, 7, 8, 9, 10, 11, 12, 13, 14, 15, 16, 17, 18, 19, 20, copies per cell after at least 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 290, 294, 300, 350, 400, 450, or 500 doublings. In some embodiments, the average copy number of backbone sequence after separation of the vector sequence from the backbone sequence is less than 5, 4, 3, 2, 1, 0.5, 0.1, 0.01, or 0.001 copies per cell or is undetectable after at most 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 290, 294, 300, 350, 400, 450, or 500 doublings (e.g., less than 0.001 copies per cell after at most 1 doubling (e.g., less than 0.001 copies per cell after at most 10 doublings, less than 0.001 copies per cell after at most 20 doublings, less than 0.001 copies per cell after at most 50 doublings, or less than 0.001 copies per cell after at most 500 doublings), less than 0.01 copies per cell after at most 1 doubling (e.g., less than 0.01 copies per cell after at most 10 doublings, less than 0.01 copies per cell after at most 20 doublings, less than 0.01 copies per cell after at most 50 doublings, or less than 0.01 copies per cell after at most 100 doublings), less than 0.1 copies per cell after at most 1 doubling (e.g., less than 0.1 copies per cell after at most 10 doublings, less than 0.1 copies per cell after at most 20 doublings, less than 0.1 copies per cell after at most 50 doublings, or less than 0.1 copies per cell after at most 100 doublings), or less than 1 copy per cell after at most 1 doubling (e.g., less than 1 copy per cell after at le most at 10 doublings, less than 1 copy per cell after at most 20 doublings, less than 1 copy per cell after at most 50 doublings, or less than 1 copy per cell after at most 100 doublings)).

Some embodiments include a culture of engineered bacterial cells in which the average copy number of a circular DNA vector or parental plasmid is at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 copies per cell, or is between any two of these values. In some embodiments, the culture comprises at least 10⁵, 10⁶, 10⁷, 10⁸, 10⁹, 10¹⁰, 10¹¹, or 10¹² total cells, or between any two of these values. In some embodiments, the culture comprises at least 10⁴, 10⁵, 10⁶, 10⁷, 10⁸, 10⁹, or 10¹⁰ cells/ml, or between any two of these values.

### E. Recovery of Circular DNA Vector

Circular DNA vector produced by embodiments disclosed herein can be recovered from a culture of engineered bacteria by extraction and purification procedures known in the art. In some embodiments, at least 0.001, 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, or 5.0 mg of circular DNA vector can be recovered per liter of cultured engineered bacterial cells. In some embodiments, the circular DNA vector goes through purification procedures to reduce the amount of bacterial contaminants, such as endotoxin, to levels acceptable for use in a pharmaceutical composition. Suitable purification procedures include chromatography procedures, such as anion exchange chromatography or hydrophobic interaction chromatography.

In some embodiments, the circular DNA vector is purified by gel electrophoresis to further avoid contamination by backbone sequence that may be maintained in a culture of engineered bacterial cells. In some embodiments, no purification is necessary to avoid detectable contamination of the circular DNA vector with backbone sequence.

In some embodiments, the circular DNA vector can be purified from a culture of engineered bacterial cells described herein without contamination of the purified product by backbone sequence or by any other extrachromosomal DNA molecules. In some embodiments, a composition of isolated circular DNA vector purified from engineered bacterial cells disclosed herein includes less than 10, 1, 0.1, 0.01, 0.001, or 0.0001 ng/ml of DNA comprising backbone sequences. In some embodiments, DNA comprising backbone sequence is undetectable in the composition by quantitative PCR. In some embodiments, these purity levels are achieved without a gel purification or column purification step being performed after isolation of the circular DNA vector from the engineered bacterial cells.

In some embodiments, methods of making circular DNA vector disclosed herein comply with current good manufacturing practice (GMP) according to the standards promulgated by the U.S. Food & Drug Administration and set forth in 21 C.F.R. Parts 210 and 211, which are incorporated herein by reference in their entirety.

### III. Circular DNA Vectors

Provided herein are circular DNA vectors produced by any of the methods of production described herein. In some instances, such circular DNA vectors persist intracellularly (e.g., in dividing or in quiescent cells, such as post-mitotic cells) as episomes, e.g., in a manner similar to AAV vectors. In any of the embodiments, described herein, a circular DNA vector may be a non-integrating vector. circular DNA vectors provided herein can be naked DNA vectors, devoid of components inherent to viral vectors (e.g., viral proteins) and bacterial plasmid DNA, such as immunogenic components (e.g., immunogenic bacterial signatures (e.g., CpG islands or CpG motifs)) or components additionally, or otherwise associated with reduced persistence (e.g., CpG islands or CpG motifs). The circular DNA vectors produced as described herein feature one or more therapeutic sequences and may lack plasmid backbone elements (e.g., a drug resistance gene). In some embodiments, the circular DNA vectors lack a recombination site. In some embodiments, in particular in embodiments in which recombination is used to remove the vector sequence from the plasmid backbone sequence, the circular DNA vector includes a recombination site.

In some embodiments, the vector contains DNA in which at least 50% (e.g., at least 60%, at least 70%, at least 80%, at least 90%, at least 95%, at least 97%, at least 99%, or essentially all) of the DNA lacks one or more elements of bacterial plasmid DNA, such as immunogenic components (e.g., immunogenic bacterial signatures (e.g., CpG motifs)) or components additionally or otherwise associated with reduced persistence (e.g., CpG islands). In some embodiments, at least 50% (e.g., at least 60%, at least 70%, at least 80%, at least 90%, at least 95%, at least 97%, at least 99%, or essentially all) of the DNA lacks CpG methylation. In some embodiments, the vector contains DNA in which at least 50% (e.g., at least 60%, at least 70%, at least 80%, at least 90%, at least 95%, at least 97%, at least 99%, or essentially all) of the DNA lacks bacterial methylation signatures, such as Dam methylation and Dem methylation. For example, in some embodiments, the vector contains DNA in which at least 50% (e.g., at least 60%, at least 70%, at least 80%, at least 90%, at least 95%, at least 97%, at least 99%, or essentially all) of the GATC sequences are unmethylated (e.g., by Dam methylase). Additionally, or alternatively, the vector contains DNA in which at least 50% (e.g., at least 60%, at least 70%, at least 80%, at least 90%, at least 95%, at least 97%, at least 99%, or essentially all) of the CCAGG sequences and/or CCTGG sequences are unmethylated (e.g., by Dem methylase).

In some embodiments, the circular DNA vector is persistent in vivo (e.g., the circular DNA vector exhibits improved expression persistence (e.g., intra-cellular persistence and/or trans-generational persistence) and/or therapeutic persistence relative to a reference vector, e.g., a circular DNA vector produced in bacteria or having one or more bacterial signatures not present in the vector of the invention, e.g., plasmid DNA). In some embodiments, expression persistence of the circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, intra-cellular persistence of the circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, trans-generational persistence of the circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, therapeutic persistence of the circular DNA vector is 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector. In some embodiments, the reference vector is a circular vector or plasmid that (a) has the same coding sequence as a circular DNA vector to which it is being compared, and (b) has one or more bacterial signatures not present in the circular DNA vector to which it is being compared, which signatures may include, for example, an antibiotic resistance gene or other selectable marker.

In some embodiments, expression of a circular DNA vector persists for one week, two weeks, three weeks, four weeks, six weeks, two months, three months, four months, five months, six months, seven months, eight months, nine months, ten months, eleven months, one year, or longer after administration. In particular embodiments, the circular DNA vector exhibits intra-cellular persistence and/or trans-generational persistence of one week, two weeks, three weeks, four weeks, six weeks, two months, three months, four months, five months, six months, seven months, eight months, nine months, ten months, eleven months, one year, or longer after administration. In some embodiments, therapeutic persistence of a circular DNA vector endures for one week, two weeks, three weeks, four weeks, six weeks, two months, three months, four months, five months, six months, seven months, eight months, nine months, ten months, eleven months, one year, or longer after administration.

In some embodiments, expression and/or therapeutic effect of the circular DNA vector persists for one week to four weeks, from one month to four months, or from four months to one year (e.g., at least one week, at least two weeks, at least one month, or longer). In some embodiments, the expression level of the circular DNA vector does not decrease by more than 90%, by more than 50%, or by more than 10% in the 1 week or more, e.g., 2 weeks, 3 weeks, 5 weeks, 7 weeks, 9 weeks or more, 13 weeks or more, 18 weeks or more following transfection from levels observed within the first 1, 2, or 3 days.

The circular DNA vector may be monomeric, dimeric, trimeric, tetrameric, pentameric, hexameric, etc. In some preferred embodiments, the circular DNA vector is monomeric. In some embodiments, the DNA vector is supercoiled. The circular DNA vector may be supercoiled due to the endogenous processes within the engineered bacterial cell or due to treatment with a topoisomerase (e.g., gyrase). In some embodiments, the circular DNA vector is a monomeric, supercoiled circular DNA molecule. In some embodiments, the circular DNA vector is nicked. In some embodiments, the circular DNA vector is open circular. In some embodiments, the circular DNA vector is double-stranded circular. In some embodiments, a composition comprising the circular DNA vector comprises at least about 70, 75, 80, 85, 90, 91, 92, 93, 94, 95, 96, 97, 98, 99, or 99.9% supercoiled monomer. In some embodiments, a composition comprising the circular DNA vector comprises at least about 70, 75, 80, 85, 90, 91, 92, 93, 94, 95, 96, 97, 98, 99, or 99.9% supercoiled monomer without treatment by an exogenous topoisomerase.

### IV. Therapeutic Sequences

In some embodiments, coding sequences of circular DNA vectors described herein contain a therapeutic sequence, which may include one or more protein-coding domains and/or one or more non-protein coding domains (e.g., a therapeutic nucleic acid).

In particular embodiments involving a therapeutic protein-coding therapeutic domain, the therapeutic sequence includes, linked in the 5′ to 3′ direction: a promoter and a single therapeutic protein-coding domain (e.g., a single transcription unit); a promoter and two or more therapeutic protein-coding domains (e.g., a multicistronic unit); or a first transcription unit and one or more additional transcription units (e.g., a multi-transcription unit). Any such protein-coding therapeutic sequences may further include non-protein coding domains, such as polyadenylation sites, control elements, enhancers, sequences to mark DNA (e.g., for antibody recognition), PCR amplification sites, sequences that define restriction enzyme sites, site-specific recombinase recognition sites, sequences that are recognized by a protein that binds to and/or modifies nucleic acids, linkers, splice sites, pre-mRNA binding domains, regulatory sequences, and/or a therapeutic nucleic acid (e.g., a microRNA-encoding sequence). Therapeutic protein-coding domains can be full-length protein-coding domains (e.g., corresponding to a native gene or natural variant thereof) or a functional portion thereof, such as a truncated protein-coding domain (e.g., minigene).

In some embodiments, the therapeutic sequence encodes a monomeric protein (e.g., a monomeric protein with secondary structure under physiological conditions, e.g., a monomeric protein with secondary and tertiary structure under physiological conditions, e.g., a monomeric protein with secondary, tertiary, and quaternary structure under physiological conditions). Additionally, or alternatively, the therapeutic sequence may encode a multimeric protein (e.g., a dimeric protein (e.g., a homodimeric protein or heterodimeric protein), a trimeric protein, etc.).

In particular instances, the therapeutic sequence includes an ocular gene. In some embodiments, the ocular gene is a gene that is expressed in ocular tissue, such as, for example retinal tissue, which may include, for example, photoreceptor cells and/or retinal pigment epithelial (RPE) cells. In some embodiments, the coding sequence in expression constructs disclosed herein is a human ABCA4 or MYO7A gene sequence. An exemplary human ABCA4 gene sequence is provided as National Center for Biotechnology Information (NCBI) Reference Sequence: NG_009073. The amino acid sequence of an exemplary ABCA4 protein is given by Protein Accession No. P78363.3. An exemplary human MYO7A gene sequence is provided as NCBI Gene ID: 4647. The amino acid sequence of an exemplary MYO7A protein is given by Protein Accession No. Q13402.

In some embodiments, the therapeutic sequence encodes an antibody, or a portion, fragment, or variant thereof. Antibodies include fragments that are capable of binding to an antigen, such as Fv, single-chain Fv (scFv), Fab, Fab′, di-scFv, sdAb (single domain antibody), (Fab′)2 (including a chemically linked F(ab′)2), and nanobodies. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. Antibodies also include chimeric antibodies and humanized antibodies. Furthermore, for all antibody constructs provided herein, variants having the sequences from other organisms are also contemplated. Thus, if a human version of an antibody is disclosed, one of skill in the art will appreciate how to transform the human sequence-based antibody into a mouse, rat, cat, dog, horse, etc. sequence. Antibody fragments also include cither orientation of single chain scFvs, tandem di-scFv, diabodies, tandem tri-sdcFv, minibodies, nanobodies, etc. In some embodiments, such as when an antibody is an scFv, a single polynucleotide of a therapeutic gene sequence encodes a single polypeptide comprising both a heavy chain and a light chain linked together. Antibody fragments also include nanobodies (e.g., sdAb, an antibody having a single, monomeric domain, such as a pair of variable domains of heavy chains, without a light chain). Multispecific antibodies (e.g., bispecific antibodies, trispecific antibodies, etc.) are known in the art and contemplated as expression products of the therapeutic gene sequences of the present invention.

In some instances, the therapeutic sequence encodes one or more proteins (e.g., a single protein, two proteins, three proteins, four proteins, or more), each having a length of at least 25 amino acids, at least 50 amino acids, at least 100 amino acids, at least 200 amino acids, at least 500 amino acids, at least 1,000 amino acids, at least 1,500 amino acids, at least 2,000 amino acids, at least 2,500 amino acids, at least 3,000 amino acids, or more (e.g., from 25 to 5,000 amino acids, from 50 to 5,000 amino acids, from 100 to 5,000 amino acids, from 200 to 5,000 amino acids, from 500 to 5,000 amino acids, from 1,000 to 5,000 amino acids, from 1,500 to 5,000 amino acids, or from 2,000 to 5,000 amino acids; e.g., from 25 to 4,000 amino acids, from 50 to 4,000 amino acids, from 100 to 4,000 amino acids, from 200 to 4,000 amino acids, from 500 to 4,000 amino acids, from 1,000 to 4,000 amino acids, from 1,500 to 4,000 amino acids, or from 2,000 to 4,000 amino acids; e.g., from 25 to 3,000 amino acids, from 50 to 3,000 amino acids, from 100 to 3,000 amino acids, from 200 to 3,000 amino acids, from 500 to 3,000 amino acids, from 1,000 to 3,000 amino acids, from 1,500 to 3,000 amino acids, or from 2,000 to 3,000 amino acids). In embodiments in which such therapeutic sequence encodes two or more proteins, the therapeutic sequence can be a multicistronic therapeutic sequence or a multi-transcription unit therapeutic sequence. Such a multicistronic therapeutic sequence may be, for example, a tri-cistronic cassette encoding Flt3L, IL-12, and XCL1, as described herein in Example 7.

In embodiments involving a non-protein coding therapeutic sequence, the therapeutic sequence lacks a protein-coding domain (e.g., a therapeutic protein-coding domain). For instance, in some embodiments, a therapeutic sequence includes a non-protein-coding therapeutic nucleic acid, such as a short hairpin RNA (shRNA)-encoding sequence or an immune activating therapeutic nucleic acid (e.g., a TLR agonist).

In some embodiments, the therapeutic sequence or other sequence of interest (which may include non-therapeutic coding sequences such as reporter genes used for measuring expression or persistence) is from 0.1 Kb to 100 Kb in length (e.g., the sequence is from 0.2 Kb to 90 Kb, from 0.5 Kb to 80 Kb, from 1.0 Kb to 70 Kb, from 1.5 Kb to 60 Kb, from 2.0 Kb to 50 Kb, from 2.5 Kb to 45 Kb, from 3.0 Kb to 40 Kb, from 3.5 Kb to 35 Kb, from 4.0 Kb to 30 Kb, from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 0.1 Kb to 0.5 Kb, from 0.5 Kb to 1.0 Kb, from 1.0 Kb to 2.5 Kb, from 2.5 Kb to 4.5 Kb, from 4.5 Kb to 8 Kb, from 8 Kb to 10 Kb, from 10 Kb to 15 Kb, from 15 Kb to 20 Kb in length, or greater, e.g., from 0.1 Kb to 0.25 Kb, from 0.25 Kb to 0.5 Kb, from 0.5 Kb to 1.0 Kb, from 1.0 Kb to 1.5 Kb, from 1.5 Kb to 2.0 Kb, from 2.0 Kb to 2.5 Kb, from 2.5 Kb to 3.0 Kb, from 3.0 Kb to 3.5 Kb, from 3.5 Kb to 4.0 Kb, from 4.0 Kb to 4.5 Kb, from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater). In some embodiments, the therapeutic sequence is at least 10 Kb (e.g., from 10 Kb to 15 Kb, from 15 Kb to 20 Kb, or from 20 Kb to 30 Kb; e.g., from 10 Kb to 13 Kb, from 10 Kb to 12 Kb, or from 10 Kb to 11 Kb; e.g., from 10-11 Kb, from 11-12 Kb, from 12-13 Kb, from 13-14 Kb, or from 14-15 Kb). In some embodiments, the sequence is at least 1,100 bp in length (e.g., from 1,100 bp to 10,000 bp, from 1,100 bp to 8,000 bp, or from 1,100 bp to 5,000 bp in length). In some embodiments, the sequence is at least 2,500 bp in length (e.g., from 2,500 bp to 15,000 bp, from 2,500 bp to 10,000 bp, or from 2,500 bp to 5,000 bp in length; e.g., from 2,500 bp to 5,000 bp, from 5,000 bp to 7,500 bp, from 7,500 bp to 10,000 bp, from 10,000 bp to 12,500 bp, or from 12,500 bp to 15,000 bp). In some embodiments, the sequence is at least 8,000 bp, at least 9,000 bp, at least 10,000 bp, at least 11,000 bp, at least 12,000 bp at least 13,000 bp, at least 14,000 bp, at least 15,000 bp, at least 16,000 bp (e.g., 11,000 bp to 16,000 bp, 12,000 bp to 16,000 bp, 13,000 bp to 16,000 bp, 14,000 bp to 16,000 bp, or 15,000 bp to 16,000 bp). In particular embodiments, the sequence is sufficiently large to encode a protein and is not an oligonucleotide therapy (e.g., not an antisense, siRNA, shRNA therapy, etc.).

In some embodiments, the 3′ end of a sequence of interest, such as a therapeutic or non-therapeutic coding sequence, is connected to the 5′ end of an ori sequence in the circular DNA vector by a non-bacterial sequence (e.g., a recombination site, e.g., a recombination scar) of no more than 50 bp (e.g., from 3 bp to 34 bp, from 4 bp to 20 bp, from 5 bp to 12 bp, or from 6 bp to 10 bp; e.g., from 3 bp to 5 bp, from 4 bp to 6 bp, from 8 bp to 12 bp, from 12 bp to 18 bp, from 18 bp to 24 bp, from 24 bp to 30 bp, from 30 bp to 35 bp, or from 35 bp to 40 bp; e.g., 3 bp, 4 bp, 5 bp, 6 bp, 7 bp, 8 bp, 10 bp, 15 bp, 20 bp, 25 bp, 30 bp, 31 bp, 32 bp, 33 bp, 34 bp, 35 bp, 36 bp, 37 bp, 38 bp, 39 bp, 40 bp, 41 bp, 42 bp, 43 bp, 44 bp, 45 bp, 46 bp, 47 bp, 48 bp, 49 bp, or 50 bp).

In some embodiments, the 3′ end of a sequence of interest, such as a therapeutic or non-therapeutic coding sequence, is connected to the 5′ end of an ori sequence in the circular DNA vector by a non-bacterial sequence of no more than 30 bp (e.g., from 3 bp to 24 bp, from 4 bp to 18 bp, from 5 bp to 12 bp, or from 6 bp to 10 bp; e.g., from 3 bp to 5 bp, from 4 bp to 6 bp, from 8 bp to 12 bp, from 12 bp to 18 bp, from 18 bp to 24 bp, or from 24 bp to 30 bp; e.g., 3 bp, 4 bp, 5 bp, 6 bp, 7 bp, 8 bp, 10 bp, 15 bp, 20 bp, 25 bp, or 30 bp).

In some embodiments, the sequence of interest included in circular DNA vectors described herein includes a reporter sequence in addition to a therapeutic protein-encoding domain or a therapeutic non-protein encoding domain. In some embodiments, the therapeutic sequence lacks a reporter sequence. In some embodiments, the sequence of interest includes a reporter sequence and does not include a therapeutic sequence. The reporter sequence can be, for example, a reporter gene. Such reporter genes can be useful in verifying therapeutic gene sequence expression, for example, in specific cells and tissues. Reporter sequences that may be provided in a circular DNA vector include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

As part of the therapeutic sequence or other sequence of interest, circular DNA vectors of the invention may include conventional control elements which modulate or improve transcription, translation, and/or expression in a target cell. Suitable control elements are described in International Publication No. WO 2021/055760, which is incorporated herein by reference in its entirety.

In some instances, a self-replicating RNA molecule includes (i) a replicase-encoding sequence (e.g., an RNA sequence that encodes an RNA-dependent RNA polymerase which can transcribe RNA from the self-replicating RNA molecule) and (ii) a heterologous modulatory gene. The polymerase can be an alphavirus replicase, e.g., an alphavirus replicase comprising one, two, three, or all four alphavirus nonstructural proteins nsP1, nsP2, nsP3, and nsP4. In some instances, the polymerase is a VEE replicase, e.g., a VEE replicase comprising one, two, three, or all four alphavirus nonstructural proteins nsP1, nsP2, nsP3, and nsP4.

In some instances of the present invention, a self-replicating RNA molecule does not encode alphavirus structural proteins (e.g., capsid proteins). Such self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins can be replaced by gene(s) encoding the heterologous modulatory protein(s) of interest, such that the subgenomic transcript encodes the heterologous modulatory protein(s) rather than the structural alphavirus virion proteins.

Accordingly, in some instances, a self-replicating RNA molecule of the invention can have two open reading frames. The first (S′) open reading frame encodes a replicase; the second (3′) open reading frame encodes one or more (e.g., two or three) therapeutic proteins. In some embodiments, the RNA may have additional (e.g., downstream) open reading frames, e.g., to encode further genes or to encode accessory polypeptides.

Suitable self-replicating RNA molecules can have various lengths. In some embodiments of the invention, the length of the self-replicating RNA molecule is from 5,000 to 50,000 nucleotides (i.e., 5 kb to 50 kb). In some instances, the self-replicating RNA molecule is 5 kb to 20 kb in length (e.g., from 6 kb to 18 kb, from 7 kb to 16 kb, from 8 kb to 14 kb, or from 9 kb to 12 kb in length, e.g., from 5 kb to 6 kb, from 6 kb to 7 kb, from 7 kb to 8 kb, from 8 kb to 9 kb, from 9 kb to 10 kb, from 10 kb to 11 kb, from 11 kb to 12 kb, from 12 kb to 13 kb, from 13 kb to 14 kb, from 14 kb to 15 kb, from 15 kb to 16 kb, from 16 kb to 18 kb, or from 18 kb to 20 kb in length, e.g., about 5 kb, about 6 kb, about 7 kb, about 8 kb, about 9 kb, about 10 kb, about 10.5 kb, about 11 kb, about 11.5 kb, about 12 kb, about 12.5 kb, about 13 kb, about 14 kb, about 15 kb, about 16 kb, about 17 kb, about 18 kb, about 19 kb, or about 20 kb in length).

A self-replicating RNA molecule may have a 3′ poly-A tail. Additionally, the self-replicating RNA molecule may include a poly-A polymerase recognition sequence (e.g., AAUAAA).

In a particular embodiment, the RNA according to the invention does not encode a reporter molecule, such as luciferase or a fluorescent protein, such as green fluorescent protein (GFP).

In some embodiments, the replicase encoded by the self-replicating RNA can be a variant of any of the replicases described herein. In some embodiments, the variant is a functional fragment (e.g., a fragment of the protein that is functionally similar or functionally equivalent to the protein).

### V. Pharmaceutical Compositions

Improvements in efficiency render the present methods particularly amenable to scalable manufacturing of pharmaceutical compositions containing circular DNA vectors. Any of the methods of producing circular DNA vectors described herein can be adapted for production of pharmaceutical compositions containing the circular DNA vector in a pharmaceutically acceptable carrier.

As part of any of the methods described herein, downstream purification processes can be readily applied. For instance, various chromatography steps are known in the art and can be suitably adapted for removal of bacterial byproducts, endotoxin, bacterial artificial chromosomes (BAC), helper plasmids, etc. In some instances, pharmaceutical compositions of bacterially produced circular DNA vectors are purified by anion exchange chromatography or hydrophobic interaction chromatography.

In some embodiments, a pharmaceutical formulation of the invention contains at least 1.0 mg circular DNA vector in a pharmaceutically acceptable carrier (e.g., from 1.0 mg to 10 g, from 1.0 mg to 5.0 g, from 1.0 mg to 1.0 g, from 1.0 mg to 500 mg, from 1.0 mg to 200 mg, from 1.0 mg to 100 mg, from 1.0 mg to 50 mg, from 1.0 mg to 25 mg, from 1.0 mg to 20 mg, from 1.0 mg to 15 mg, from 1.0 mg to 10 mg, from 1.0 mg to 5.0 mg, from 2.0 mg to 10 g, from 2.0 mg to 5.0 g, from 2.0 mg to 1.0 g, from 2.0 mg to 500 mg, from 2.0 mg to 200 mg, from 2.0 mg to 100 mg, from 2.0 mg to 50 mg, from 2.0 mg to 25 mg, from 2.0 mg to 20 mg, from 2.0 mg to 15 mg, from 2.0 mg to 10 mg, from 2.0 mg to 5.0 mg, from 5.0 mg to 10 g, from 5.0 mg to 5.0 g, from 5.0 mg to 1.0 g, from 5.0 mg to 500 mg, from 5.0 mg to 200 mg, from 5.0 mg to 100 mg, from 5.0 mg to 50 mg, from 5.0 mg to 25 mg, from 5.0 mg to 20 mg, from 5.0 mg to 15 mg, from 5.0 mg to 10 mg, from 10 mg to 10 g, from 10 mg to 5.0 g, from 10 mg to 1.0 g, from 10 mg to 500 mg, from 10 mg to 200 mg, from 10 mg to 100 mg, from 10 mg to 50 mg, from 10 mg to 25 mg, from 10 mg to 20 mg, or from 10 mg to 15 mg).

In some embodiments, a pharmaceutical formulation of the invention contains at least 2.0 mg circular DNA vector in a pharmaceutically acceptable carrier. In some embodiments, a pharmaceutical formulation produced by any of the methods described herein contains at least 5.0 mg circular DNA vector in a pharmaceutically acceptable carrier. In some embodiments, a pharmaceutical formulation produced by any of the methods described herein contains at least 10.0 mg circular DNA vector in a pharmaceutically acceptable carrier.

In some embodiments, the pharmaceutical formulation of the invention is substantially devoid of impurities. For instance, in some embodiments, the pharmaceutical formulation contains <2.0% protein content by mass (e.g., <1.9%, <1.8%, <1.7%, <1.6%, <1.5%, <1.4%, <1.3%, <1.2%, <1.1%, <1.0%, <0.9%, <0.8%, <0.7%, <0.6%, <0.5%, <0.4%, <0.3%, <0.2%, <0.1%, <0.05%, or <0.01% protein content by mass). In some instances, protein content is determined by bicinchoninic acid assay. Additionally or alternatively, protein content is determined by ELISA.

In some instances, the pharmaceutical formulation contains <5.0% RNA content by mass (e.g., <4.5%, <4.0%, <3.5%, <3.0%, <2.5%, <2.0%, <1.9%, <1.8%, <1.7%, <1.6%, <1.5%, <1.4%, <1.3%, <1.2%, <1.1%, <1.0%, <0.9%, <0.8%, <0.7%, <0.6%, <0.5%, <0.4%, <0.3%, <0.2%, <0.1%, <0.05%, or <0.01% RNA content by mass). In some embodiments, the RNA content is determined by agarose gel electrophoresis. In some embodiments, the RNA content is determined by quantitative PCR. In some embodiments, the RNA content is determined by fluorescence assay (e.g., Ribogreen).

In some embodiments, the pharmaceutical formulation contains <5.0% gDNA content by mass (e.g., <4.5%, <4.0%, <3.5%, <3.0%, <2.5%, <2.0%, <1.9%, <1.8%, <1.7%, <1.6%, <1.5%, <1.4%, <1.3%, <1.2%, <1.1%, <1.0%, <0.9%, <0.8%, <0.7%, <0.6%, <0.5%, <0.4%, <0.3%, <0.2%, <0.1%, <0.05%, or <0.01% gDNA content by mass). In some embodiments, the gDNA content is determined by agarose gel electrophoresis or capillary electrophoresis. In some embodiments, the gDNA content is determined by quantitative PCR. In some embodiments, the gDNA content is determined by Southern blot.

In some embodiments, the pharmaceutical formulation contains <40 EU/mg endotoxin. In some embodiments, the pharmaceutical formulation contains <20 EU/mg endotoxin. In some embodiments, the pharmaceutical formulation contains <10 EU/mg endotoxin. In some embodiments, the pharmaceutical formulation contains <5 EU/mg endotoxin (e.g., <4 EU/mg endotoxin, <3 EU/mg endotoxin, <2 EU/mg endotoxin, <1 EU/mg endotoxin, <0.5 EU/mg endotoxin), e.g., as measured by Limulus Amebocyte Lysate (LAL) assay.

In some embodiments, pharmaceutical compositions disclosed herein comply with current good manufacturing practice (GMP) according to the standards promulgated by the U.S. Food & Drug Administration and set forth in 21 C.F.R. Parts 210 and 211, which are incorporated herein by reference in their entirety.

Pharmaceutical compositions provided herein may include one or more pharmaceutically acceptable carriers, such as excipients and/or stabilizers that are nontoxic to the individual being treated (e.g., human patient) at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

If the pharmaceutical composition is provided in liquid form, the pharmaceutically acceptable carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl, CaI₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂)) or potassium chloride (KCl), wherein further anions may be present. CaCl₂) can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂)). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from theobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers; antioxidants; and preservatives.

The pharmaceutical composition according to the present invention may be provided in liquid or in dry (e.g., lyophilized) form. In a particular embodiment, the nucleic acid vector of the pharmaceutical composition is provided in lyophilized form. Lyophilized compositions including nucleic acid vector of the invention may be reconstituted in a suitable buffer, advantageously based on an aqueous carrier, prior to administration, e.g., Ringer-Lactate solution, Ringer solution, or a phosphate buffer solution.

In certain embodiments of the invention, any of the circular DNA vectors of the invention can be complexed with one or more cationic or polycationic compounds, e.g., cationic or polycationic polymers, cationic or polycationic peptides or proteins, e.g. protamine, cationic or polycationic polysaccharides, and/or cationic or polycationic lipids.

According to a particular embodiment, the circular DNA vector of the invention may be complexed with lipids to form one or more liposomes, lipoplexes, or lipid nanoparticles. Therefore, in one embodiment, the pharmaceutical composition comprises liposomes, lipoplexes, and/or lipid nanoparticles comprising a circular DNA vector.

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems for circular DNA vectors. Cationic lipids, such as MAP, (1,2-dioleoyl-3-trimethylammonium-propane) and DOTMA (N-[1-(2,3-dioleoyloxy) propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

Thus, in one embodiment of the invention, a circular DNA vector is complexed with cationic lipids and/or neutral lipids and thereby forms liposomes, lipid nanoparticles, lipoplexes or neutral lipid-based nanoliposomes in the present pharmaceutical compositions.

In a particular embodiment, a pharmaceutical composition comprises the circular DNA vector of the invention that is formulated together with a cationic or polycationic compound and/or with a polymeric carrier. Accordingly, in a further embodiment of the invention, the circular DNA vector as defined herein is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4:1 (w/w) to about 1:1 (w/w) or of about 3:1 (w/w) to about 1:1 (w/w), e.g., from about 3:1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the circular DNA vector to the one or more polycations is in the range of about 0.1 to 10, including a range of about 0.3 to 4, of about 0.5 to 2, of about 0.7 to 2 and of about 0.7 to 1.5.

The nucleic acid vectors described herein can also be associated with a vehicle, transfection or complexation agent for increasing the transfection efficiency and/or the expression of the modulatory gene according to the invention.

In some instances, the circular DNA vector according to the invention is complexed with one or more polycations, preferably with protamine or oligofectamine. Further cationic or polycationic compounds, which can be used as transfection or complexation agent may include cationic polysaccharides, for example chitosan, polybrene, cationic polymers, e.g. polyethyleneimine (PEI), cationic lipids, e.g. DOTMA: [1-(2,3-siolcyloxy) propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP: dioleoyloxy-3-(trimethylammonio) propane, DC-6-14: O,O-ditetradecanoyl-N-(α-trimethylammonioacetyl) diethanolamine chloride, CLIP1: rac-[(2,3-dioctadecyloxypropyl) (2-hydroxyethyl)]-dimethylammonium chloride, CLIP6: rac-[2 (2,3-dihexadecyloxypropyl-oxymethyloxy)ethyl]trimethylammonium, CLIP9: rac-[2 (2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., block polymers consisting of a combination of one or more cationic blocks (e.g., selected from a cationic polymer as mentioned above) and of one or more hydrophilic or hydrophobic blocks (e.g. polyethyleneglycole); etc.

According to a particular embodiment, the pharmaceutical composition includes the circular DNA vector encapsulated within or attached to a polymeric carrier. A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herein by reference. In this context, the cationic components that form basis for the polymeric carrier by disulfide-crosslinkage are typically selected from any suitable cationic or polycationic peptide, protein or polymer suitable for this purpose, particular any cationic or polycationic peptide, protein or polymer capable of complexing the nucleic acid vector as defined herein or a further nucleic acid comprised in the composition, and thereby preferably condensing the nucleic acid vector. The cationic or polycationic peptide, protein or polymer, may be a linear molecule; however, branched cationic or polycationic peptides, proteins or polymers may also be used.

Every disulfide-crosslinking cationic or polycationic protein, peptide or polymer of the polymeric carrier, which may be used to complex the circular DNA vector according to the invention included as part of the pharmaceutical composition may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the circular DNA vector of the present invention may be formed by disulfide-crosslinked cationic (or polycationic) components. In particular, such cationic or polycationic peptides or proteins or polymers of the polymeric carrier, which comprise or are additionally modified to comprise at least one SH moiety, can be selected from proteins, peptides, and polymers as a complexation agent.

In other embodiments, the circular DNA vector according to the invention may be administered naked in a suitable buffer without being associated with any further vehicle, transfection, or complexation agent.

### VI. Methods of Use

Provided herein are methods of inducing expression (e.g., persistent expression) of a sequence of interest (e.g., a therapeutic sequence) in a subject in need thereof (e.g., as part of a gene therapy regimen) by administering to the subject any of the circular DNA vectors, or pharmaceutical compositions thereof, described herein. Target cells or tissues of a subject can be characterized by examining a nucleic acid sequence (e.g., an RNA sequence, e.g., an mRNA sequence) of the host cell, such as by Southern Blotting or PCR analysis, to detect or quantify the presence (e.g., persistence) of the therapeutic sequence delivered. Alternatively, expression of the therapeutic sequence in the subject can be characterized (e.g., quantitatively or qualitatively) by monitoring the progress of a disease being treated by delivery of the therapeutic sequence (e.g., associated with a defect or mutation targeted by the therapeutic sequence). In some embodiments, transcription or expression (e.g., persistent transcription or persistent expression) of the therapeutic sequence is confirmed by observing a decline in one or more symptoms associated with the disease.

Accordingly, embodiments of the invention include methods of treating a disease in a subject by administering to the subject any of the circular DNA vectors, or pharmaceutical compositions thereof, described herein. Any of the circular DNA vectors, or pharmaceutical compositions thereof, described herein can be administered to a subject in a dosage from 1 μg to 10 mg of DNA (e.g., from 5 μg to 5.0 mg, from 10 μg to 2.0 mg, or from 100 μg to 1.0 mg of DNA, e.g., from 10 μg to 20 μg, from 20 μg to 30 μg, from 30 μg to 40 μg, from 40 μg to 50 μg, from 50 μg to 75 μg, from 75 μg to 100 μg, from 100 μg to 200 μg, from 200 μg to 300 μg, from 300 μg to 400 μg, from 400 μg to 500 μg, from 500 μg to 1.0 mg, from 1.0 mg to 5.0 mg, or from 5.0 mg to 10 mg of DNA, e.g., about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 600 μg, about 700 μg, about 750 μg, about 1.0 mg, about 2.0 mg, about 2.5 mg, about 5.0 mg, about 7.5 mg, or about 10 mg of DNA).

In some embodiments, administration of a circular DNA vector, or a pharmaceutical composition thereof, is less likely to induce an immune response in a subject compared with administration of other gene therapy vectors (e.g., plasmid DNA vectors and viral vectors).

In some instances, the circular DNA vectors, and pharmaceutical compositions thereof, provided herein are amenable to repeat dosing due to their ability to transfect target cells without triggering an immune response or inducing a reduced immune response relative to a reference vector, such as a plasmid DNA vector or an AAV vector, as discussed above. Thus, the invention provides methods of repeatedly administering the circular DNA vectors and pharmaceutical compositions described herein. Any of the aforementioned dosing quantities may be repeated at a suitable frequency and duration. In some embodiments, the subject receives a dose about twice per day, about once per day, about five times per week, about four times per week, about three times per week, about twice per week, about once per week, about twice per month, about once per month, about once every six weeks, about once every two months, about once every three months, about once every four months, twice per year, once yearly, or less frequently. In some embodiments, the number and frequency of doses corresponds with the rate of turnover of the target cell. It will be understood that in long-lived post-mitotic target cells transfected using the vectors described herein, a single dose of vector may be sufficient to maintain expression of the heterologous gene within the target cell for a substantial period of time. Thus, in other embodiments, a circular DNA vector provided herein may be administered to a subject in a single dose. The number of occasions in which a circular DNA vector is delivered to the subject can be that which is required to maintain a clinical (e.g., therapeutic) benefit.

Methods of the invention include administration of a circular DNA vector or pharmaceutical composition thereof through any suitable route. The circular DNA vector or pharmaceutical composition thereof can be administered systemically or locally, e.g., intravenously, ocularly (e.g., intravitreally (e.g., by intravitreal injection), subretinally, by eye drop, intraocularly, intraorbitally), intramuscularly, intradermally, intrahepatically, intracerebrally, intramuscularly, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, orally, topically, transdermally, by inhalation, by aerosolization, by injection (e.g., by jet injection), by electroporation, by implantation, by infusion (e.g., by continuous infusion), by localized perfusion bathing target cells directly, by catheter, by lavage, in creams, or in lipid compositions.

Circular DNA vectors described herein can be delivered into cells via in vivo electrotransfer (e.g., in vivo electroporation). In vivo electroporation has been demonstrated in certain tissues, such as skin, skeletal muscle, certain tumor types, and lung epithelium. Delivery of naked DNA into cells by in vivo electroporation involves administration of the DNA into target tissue, followed by application of electrical field to temporarily increase cell membrane permeability within the tissue by generating pores, allowing the DNA molecules to cross cell membranes. As an example, delivery to skin using in vivo electroporation is described in Cha & Daud Hum. Vaccin. Immunother. 2012, 8 (11): 1734-1738, which is incorporated by reference in its entirety. In vivo electroporation of skeletal muscle is described in Sokolowska & Blachnio-Zabielska, Int. J. Molecular Sci. 2019, 20:2776, which is incorporated by reference in its entirety. Intratumoral delivery using in vivo electroporation is described in Aung et al. Gene Therapy 2009, 16:830-839, which is incorporated by reference in its entirety. In vivo electroporation of DNA into lung cells is described in Pringle et al. J. Gene Med. 2007, 9:369-380, which is incorporated by reference in its entirety. In vivo electrotransfer of circular DNA vectors to cells in the eye (e.g., retinal cells and/or photoreceptor cells) is described in International Patent Publication No. WO 2022/198138, which is incorporated by reference in its entirety. In some instances, after administration of the circular DNA vector to the eye, an electrode can be positioned within the interior of the eye (e.g., within about 1 mm from the retina), and an electric field can be transmitted through the electrode into a target ocular tissue at conditions suitable for electrotransfer of the circular DNA vector into the target cell (e.g., by applying six to ten pulses from 10-100 V each). Devices and systems having electrodes suitable for transmitting electric fields in mammalian tissues are commercially available and can be useful in the methods disclosed herein. In some instances, the electric field is transmitted through an electrode comprising a needle (e.g., a needle positioned within the vitreous humor or in the subretinal space). Suitable needle electrodes include CLINIPORATOR® electrodes marketed by IGEA® and needle electrodes marketed by AMBU®. Methods of the invention include administration of any of the circular DNA vectors described herein, or pharmaceutical compositions thereof, to skin, skeletal muscle, tumors (including, e.g., melanomas), eye, and lung via in vivo electrotransfer.

Additionally, or alternatively, circular DNA vectors or pharmaceutical compositions thereof can be administered to host cells ex vivo, such as by cells explanted (or otherwise derived from, e.g., induced differentiation) from an individual patient, followed by reimplantation of the host cells into a patient, e.g., after selection for cells which have incorporated the vector. Thus, in some aspects, the disclosure provides transfected host cells and methods of administration thereof for treating a disease.

Additionally, or alternatively, the present invention includes methods of treating a subject having a disease or disorder by administering to the subject the isolated DNA vector (or a composition thereof) of the invention.

Assessment of the efficiency of transfection of any of the circular DNA vectors described herein can be performed using any method known in the art or described herein. Isolating a transfected cell can also be performed in accordance with standard techniques. For example, a cell comprising a therapeutic gene can express a visible marker, such as a fluorescent protein (e.g., GFP) or other reporter protein, encoded by the sequence of the heterologous gene that aids in the identification and isolation of a cell or cells comprising the heterologous gene. Cells containing a therapeutic gene can also be characterized by examining the nucleic acid sequence (e.g., an RNA sequence, e.g., an mRNA sequence) of the host cell, such as by Southern Blotting or PCR analysis, to assay for the presence of the heterologous gene contained in the vector.

Accordingly, methods of the present invention include, after administering any of the circular DNA vectors encoding a gene as described herein to a subject, subsequently detecting the expression of the gene in the subject. Expression can be detected one week to four weeks after administration, one month to four months after administration, four months to one year after administration, one year to five years after administration, or five years to twenty years after administration (e.g., at least one week, at least two weeks, at least one month, at least four months, at least one year, at least two years, at least five years, at least ten years after administration). At any of these detection timepoints, persistence (e.g., episomal persistence) of the circular DNA vector may be observed. In some embodiments, the persistence of the circular DNA vector is from 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector (e.g., a circular vector produced in bacteria or having one or more bacterial signatures not present in the vector of the invention).

### VII. Kits and Articles of Manufacture

In another aspect of the invention, described herein is an article of manufacture or a kit containing any of the circular DNA vectors, or pharmaceutical compositions thereof. The article of manufacture includes a container and a label or package insert on or associated with the container. Suitable containers include, for example, bottles, vials, syringes, IV solution bags, etc. The containers may be formed from a variety of materials, such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). At least one active agent in the composition is a circular DNA vector of the invention or a pharmaceutical composition comprising the circular DNA vector. The label or package insert indicates that the composition is used for treating the condition treatable by its contents. Moreover, the article of manufacture may comprise (a) a first container with a composition contained therein, wherein the composition comprises a circular DNA vector, or pharmaceutical composition thereof; and (b) a second container with a composition contained therein, wherein the composition comprises an additional therapeutic agent. The article of manufacture may further comprise a package insert indicating that the compositions can be used to treat a particular condition. Alternatively, or additionally, the article of manufacture may further comprise a second (or third) container comprising a pharmaceutically acceptable carrier, such as bacteriostatic water for injection (BWFI), phosphate-buffered saline, Ringer's solution, dextrose solution, or any of the pharmaceutically acceptable carriers disclosed above. It may further include other materials desirable from a commercial and user standpoint, including other buffers, diluents, filters, needles, syringes, or other delivery devices.

### EXAMPLES

### Example 1. ColE2-P9 Replication Origin Confers Stable Plasmid Maintenance

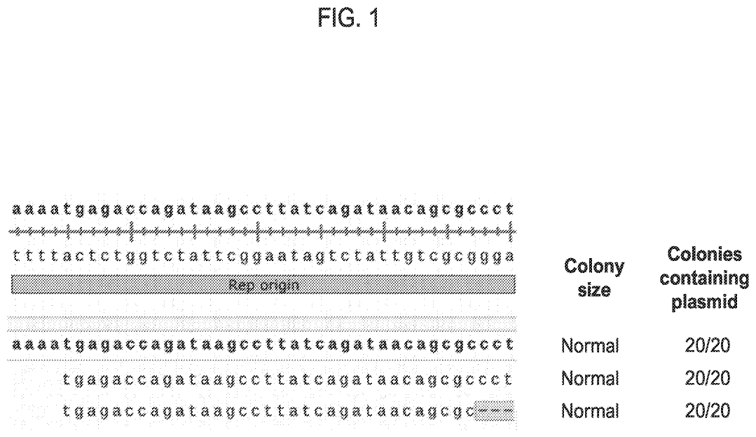

To test whether a ColE2-P9 replication origin (ori) positioned within a plasmid could confer stable maintenance of the plasmid in E. coli, plasmids containing one of three variations of the ori (SEQ ID) NOs: 2-4), an R6K origin, and a carbenicillin resistant marker were constructed and prepared in pir+ cells. Plasmids were transformed into S1037 and selected on LB agar plates supplemented with carbenicillin. A colony from each plate was cultured in LB without antibiotics at 37C. Overnight cultures were diluted 1000-fold in fresh LB without antibiotics and cultured at 37C. After five passages, each overnight culture was re-streaked on LB agar plate without antibiotics and grown overnight at 37C. From each plate, 20 colonies were plated on LB agar plate with or without carbenicillin. After six days total (during which an estimated ≥210 population doublings occurred colony size for all origin variants was normal, and plasmid was detected in all 20 colonies for all three origin variants (FIG. 1). These results demonstrate that the 40-bp replication origin allowed plasmids to remain stable over many population doublings in E. coli.

### Example 2. Production of Parental Plasmid for Production of Circular DNA Vectors

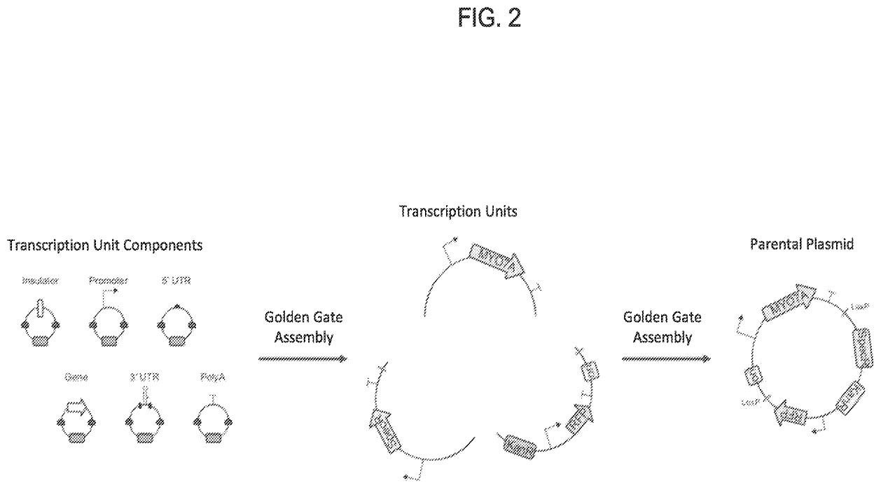

FIG. 2 shows an example of a production process for a parental plasmid that can be used to produce a circular DNA vector according to embodiments disclosed herein. Individual components of transcription units are assembled into transcription units by Golden Gate assembly. The transcription units are then assembled into a parental plasmid by Golden Gate assembly. In this example, the parental plasmid includes LoxP recombination site sequences flanking a vector sequence (the segment that includes ori and the MYO7A gene) and separating the vector sequence from a backbone sequence (the segment that includes Speck, KanR, and RFP genes).

### Example 3. In Vivo Production of Circular DNA Vector from Parental Plasmid

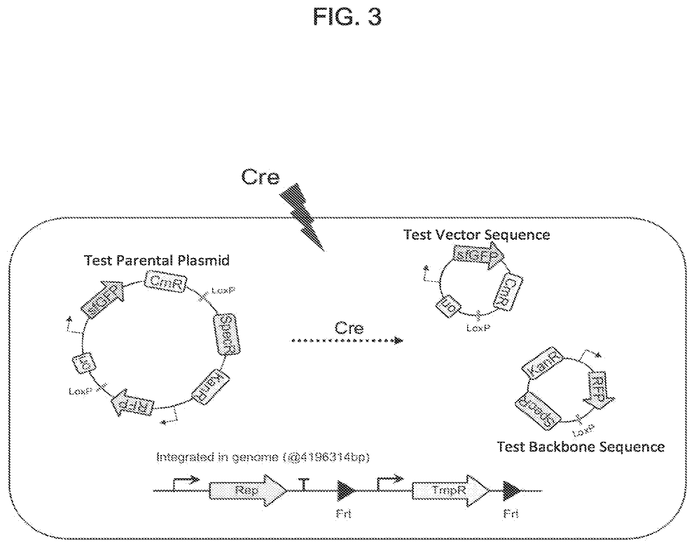

FIG. 3 shows an experimental process for in vivo production of a circular DNA vector. A test parental plasmid containing a vector sequence flanked by LoxP recombination sites is produced using a Golden Gate assembly method. The test vector sequence of the test parental plasmid includes a ColE2-P9 ori sequence and a reporter gene (sfGFP). For purposes of the experiment, the test vector sequence also includes a chloramphenicol resistance gene (CmR), although embodiments of circular DNA vectors described herein lack antibiotic resistance genes. The test parental plasmid also includes a test backbone sequence that includes antibiotic resistance genes SpecR and KanR and a reporter gene (RFP). The test parental plasmid was transformed into an engineered E. coli cell that had a ColE2-P9 Rep gene under control of a constitutive promoter (J23119) integrated into the genome. Additional versions of the test parental plasmid were made that had, instead of LoxP sites flanking the test vector sequence, two I-PpoI restriction sites, two I-SceI restriction sites, two P1-SceI restriction sites, two I-CeuI restriction sites, or one P1-PspI restriction site and one SceI restriction site flanking the test vector sequence. The test parental plasmid with LoxP sites was designated p1603, and the test parental plasmid with two P1-SceI restriction sites was designated p1600.

Bacterial colonies harboring the parental test plasmid were identified by being positive for RFP fluorescence. Cre recombinase was electroporated into cells harboring p1603 to induce recombination at the LoxP sites and produce test circular DNA vector. The procedure for Cre electroporation was as follows: Electrocompetent engineered E. coli harboring a parental plasmid cultured to OD of 0.8 in SOB at 30° C. E. coli was washed three times with ice cold 10% glycerol and resuspended in 10% glycerol. 1 μl of Cre (15 units, NEB, M0298M) was mixed with 50 μl of electrocompetent cells. The mixture was transferred to a cuvette (1 mm gap) and electroporated using an electroporator (BTX) using 1800 volt setting. The cells were rescued by growing in SOC for 1 hr at 37° C., and plated on LB agar plate without antibiotics. Colonies were grown and DNA was purified using QIAGEN miniprep kit. The electroporated cells were spread on LB plates without antibiotics. Colonies on the LB plates that were GFP-positive were streaked on LB with kanamycin (Kan) and spectinomycin (Spec) to test for loss of the test backbone sequence (Kan/Spec-sensitive colonies),

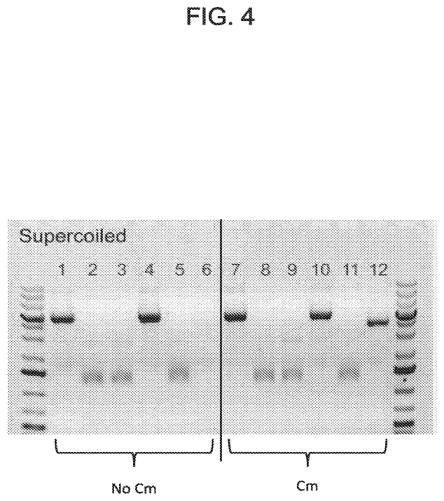

FIG. 4 shows an agarose gel electrophoresis of extragenomic DNA purified from cultures of individual colonies and shows that (1) test circular DNA vector was produced by Cre electroporation of cells harboring p1603 and (2) the test circular DNA vector was maintained in the cells in the absence of selective pressure. Lanes 2, 3, and 5, show extragenomic DNA derived from p1603-transformed cells that were GFP-positive and Kan/Spec-sensitive after Cre electroporation and that were grown in rich media lacking chloramphenicol. The bands in these lanes run at approximately 1500 bp, which is the expected size for the test circular DNA vector, showing that Cre electroporation resulted in production of test circular DNA vector. Lanes 8, 9, and 11 correspond to lanes 2, 3, and 5, respectively, but were grown in chloramphenicol-containing rich media. The abundance of DNA in lanes 2, 3, and 5, were similar to lanes 8, 9, and 11, showing that the circular DNA vector is maintained in the cells without selective pressure. Lane 4 shows extragenomic DNA derived from p1603-transformed cells that were GFP-positive, RFP-positive, and Kan/Spec-resistant after Cre electroporation. The band in this lane runs at approximately 5000 bp, which is the expected size for the test parental plasmid. Lane 10 corresponds with lane 4, but shows DNA from cells grown in chloramphenicol-containing media. Lane 1 shows extrachromosomal DNA purified from a GFP-positive, RFP-positive, Kan/Spec-resistant colony from p1600-transformed cells. The band in this lane runs at approximately 5000 bp, which is the expected size for the test parental plasmid. Lane 7 corresponds with lane 1 but shows DNA from cells grown in chloramphenicol-containing media. Lane 6 shows extrachromosomal DNA purified from the same engineered E. coli cells transformed with a plasmid lacking the ColE2-P9 ori and grown in media lacking chloramphenicol. No detectable plasmid was recovered from this culture, which may indicate that the ori is required for maintenance of the plasmid in the absence of selective pressure. Lane 12 shows DNA purified from a culture of the same cells used for lane 6 but grown in the presence of chloramphenicol. Plasmid was recovered from these cells, which may indicate that selective pressure maintained the plasmid in the cells.

### Example 4. Circular DNA Vectors with ColE2-P9 Ori are Maintained in Engineered Cells Expressing ColE2-P9 Replication Protein without Selective Pressure

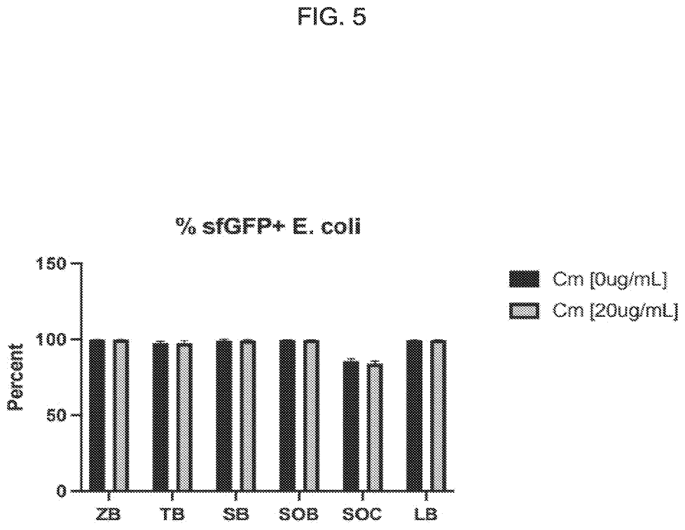

To test the ability of circular DNA vectors having an ori sequence (e.g., SEQ ID 2) from ColE2-P9 to be maintained in cells expressing ColE2-P9 replication protein (e.g., SEQ ID 1) in the absence of selective pressure, cells harboring a test circular DNA vector produced by Cre recombination as described in Example 2 were cultured in various broths with and without chloramphenicol. The percentage of sfGFP-positive cells in each culture was quantified. The results are shown in FIG. 5. The test circular DNA vector was maintained at a high level in all media with and without chloramphenicol, other than SOC media, which had lower maintenance levels both with and without chloramphenicol.

Test circular DNA vector was purified from TB and ZB cultures without chloramphenicol and quantified. The TB culture yielded 0.33 mg/L of test circular DNA vector, and the SB culture yielded 0.45 mg/L of test circular DNA vector. These results demonstrate that the circular DNA vectors were maintained regardless of selective pressure.

### Example 5. Process for In Vivo Production of Circular DNA Vector with Counterselection

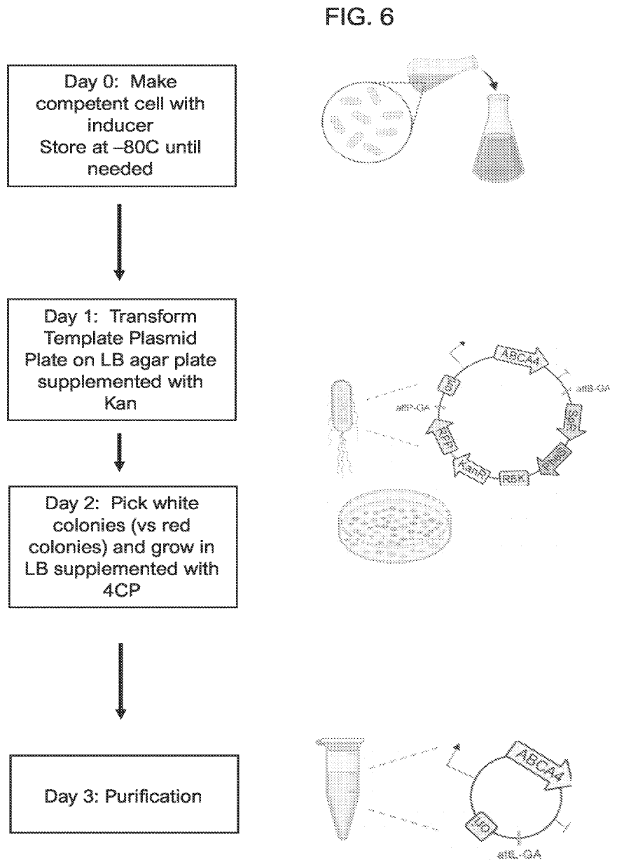

FIG. 6 shows an exemplary process of producing a circular DNA vector of the invention using counterselection. The transgene in this example is ABCA4, but it will be appreciated that a promoter driving ABCA4 could be substituted with other transgene cassettes. At day 0, competent engineered bacterial cells expressing a Rep gene are prepared using any of the processes described herein (e.g., by transforming cells with recombinase encoded on a bacterial artificial chromosome (BAC)). At day 1, cells are plated on LB agar plate supplemented with Kan, and template plasmid is added. In this example, the template plasmid included the ABCA4 transgene downstream of a promoter and a replication origin (ori). This ori-ABCA4 cassette was flanked by recombination sites (attP-GA and attB-GA). On the opposite side of the plasmid (backbone region) were selectable markers: antibiotic resistance genes SpR and KanR, counterselection marker PheS, and fluorescent marker RFP. On day 2, white colonies were picked out from the red colonies and grown in LB supplemented with 4CP for counterselection. At day 3, circular DNA vectors are purified.

### Example 6. Testing Inducible Bxb1 to Produce Circular DNA Vectors

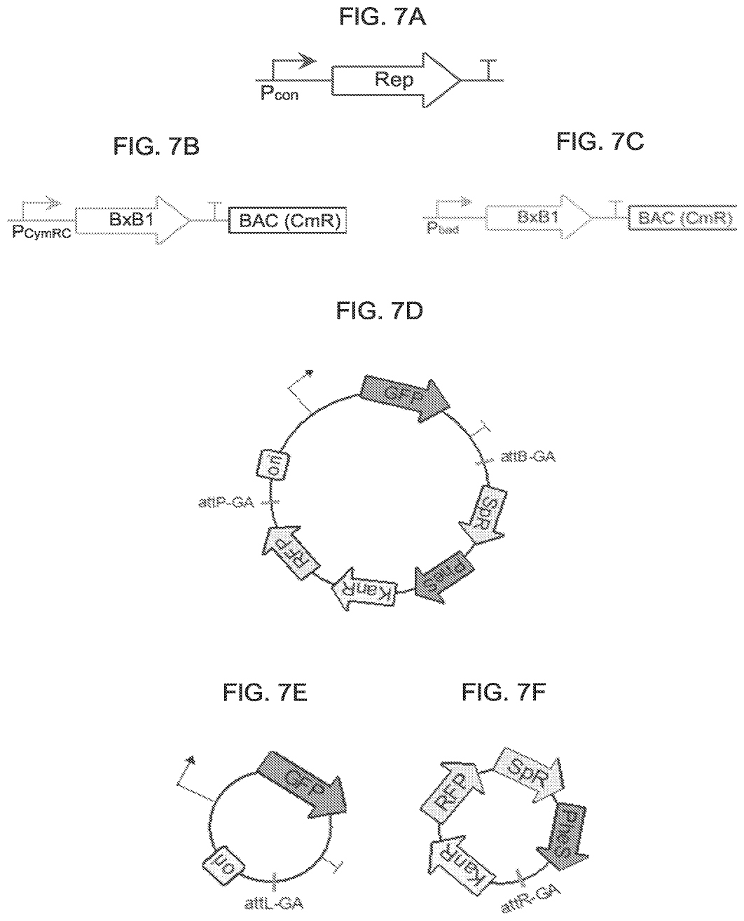

To test whether Bxb1 could be effective as an exogenous recombinase to produce circular DNA vectors, Bxb1 recombinase was encoded on a bacterial artificial chromosome (BAC) and transformed into host E. coli having a Rep gene (SEQ ID) NO: 1) integrated into its genome and driven by a constitutive promoter (FIG. 7A)). Two inducible Bxb1 BACs were tested: 1696 (FIG. 7B; SEQ ID NO: 5) included a cuminic acid inducible promoter and a chloramphenicol (Cm) resistance (CmR) gene, and 1697 (FIG. 7C; SEQ ID NO: 6) included an arabinose inducible promoter and a CmR gene. Each BAC was transformed by electroporation into S1037 cells and plated in the presence of chloramphenicol.

Next, the cells were transformed with a template plasmid carrying GFP as a reporter transgene (FIG. 7D). The ColE2-P9 replication origin (ori) was positioned upstream of GFP and its promoter, and recombination sites (attP-GA and attB-GA) flanked the ori-GFP cassette. On the opposite side of the plasmid (backbone region) were selectable markers (antibiotic resistance genes SpR and KanR, counterselection marker PheS, and fluorescent marker RFP). Thus, cells containing the template plasmid are GFP+, RFP+, Kan resistant, Spec resistant, and 4CP sensitive, whereas the cells containing only the circular DNA vector (FIG. 7E) (without the backbone byproduct (FIG. 7F)) are GFP+, RFP−, Kan-sensitive, Spec-sensitive, and 4CP resistant.

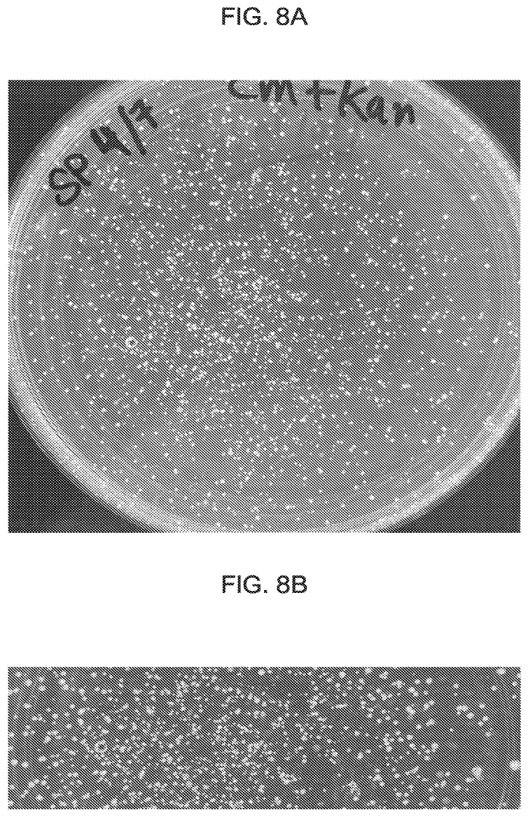

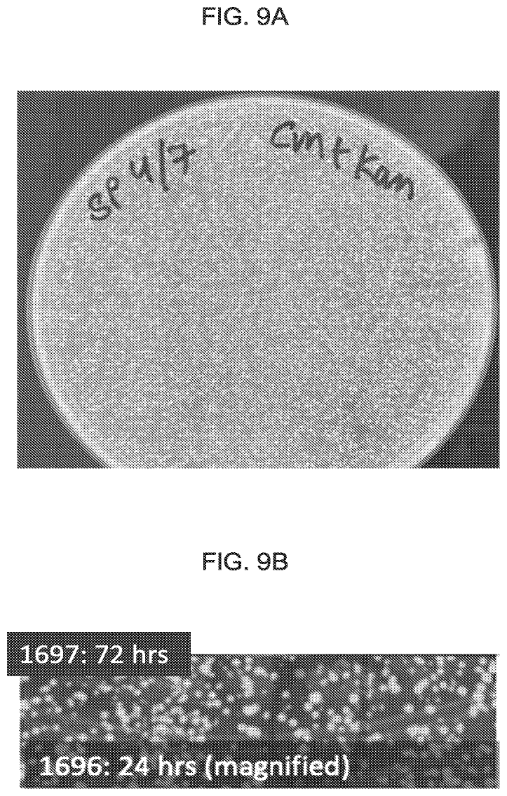

Template plasmids were electroporated into S1037 cells without inducers and plated on Cm+Kan plates. Results 24 and 72-hours post-transformation are shown in FIGS. 8 and 9. FIGS. 8A and 8B show that the majority of colonies harboring 1696 BAC were green at 24 and 72 hours post-transformation, respectively. A few red colonies were observed. Green colonies were selected and circular DNA vector presence and sequence was confirmed by Sanger sequencing and gel electrophoresis. In contrast to 1696, FIGS. 9A and 9B show that most colonies harboring 1697 BAC were yellow at 24 and 72-hours post-transformation, respectively, while a few green colonies were observed at 72 hours (FIG. 9B).

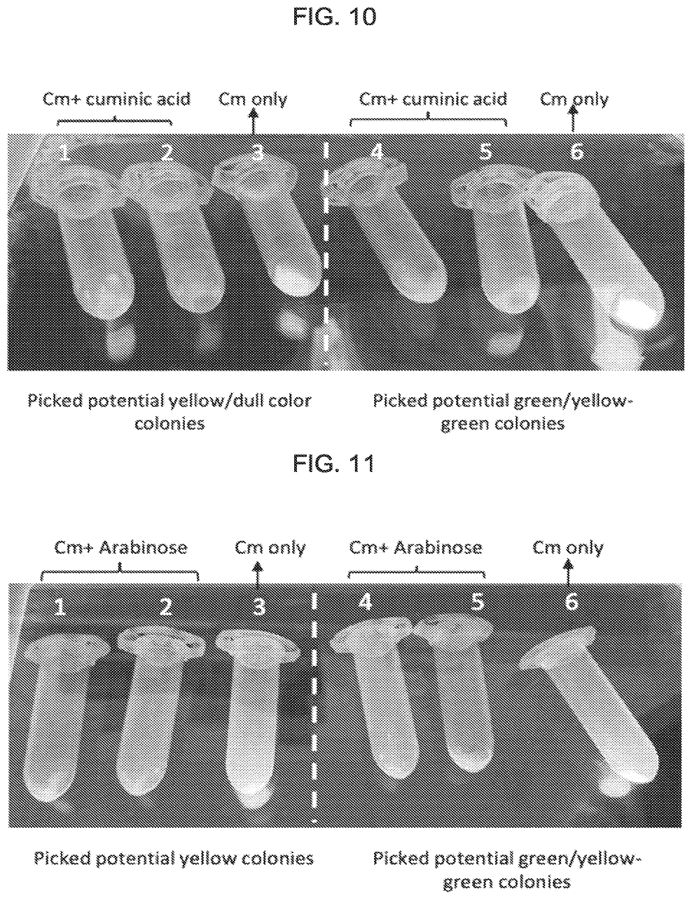

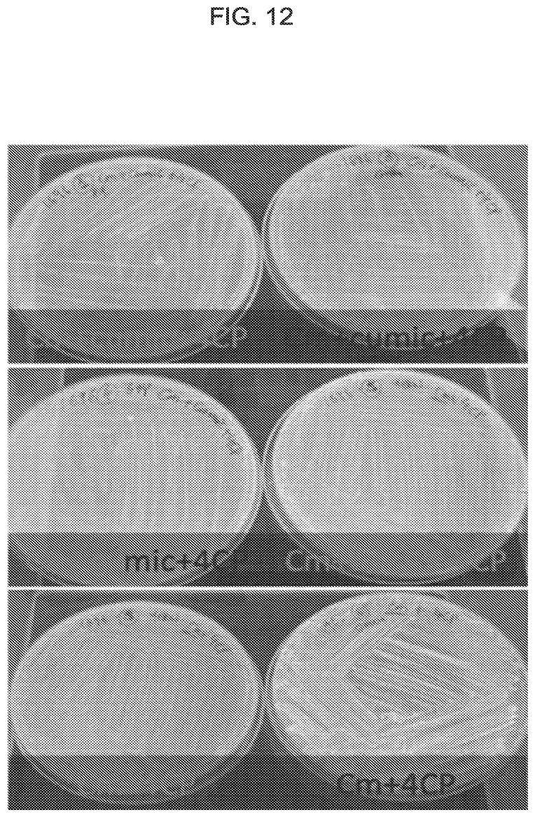

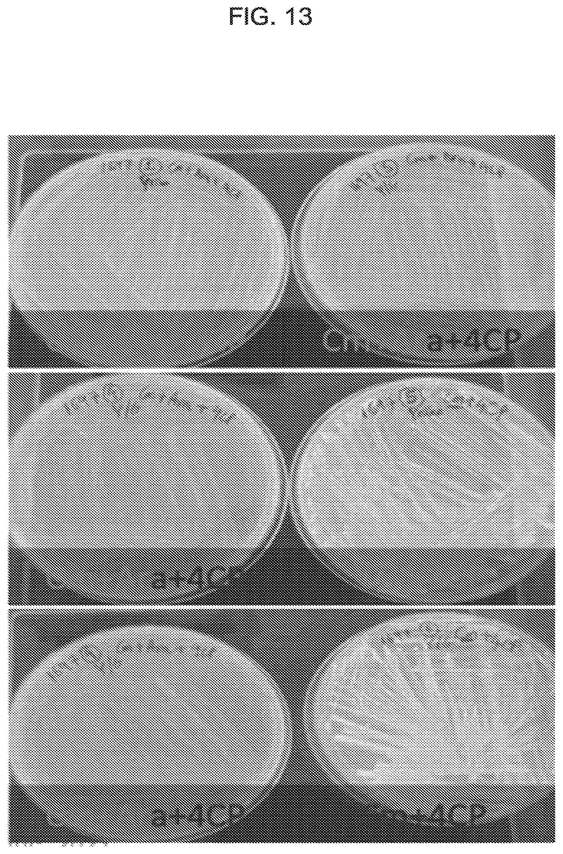

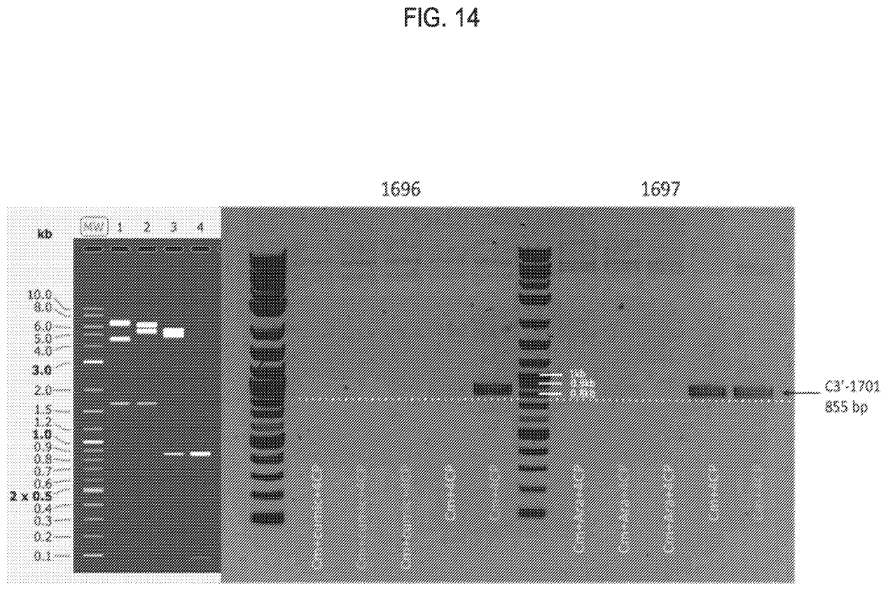

To assess the effects from Cm and arabinose inducers, various colored colonies from each plate were picked and incubated with Cm and either cuminic acid or arabinose for 24 hours. Results are shown in FIG. 10 (1696) and FIG. 11 (1697). Next, each culture was diluted 500-fold and grown overnight in LB supplemented with 4CP for counterselection, with or without inducers. Overnight cultures were re-streaked on plain LB agar plates and observed for fluorescence. Results are shown in FIGS. 12 (1696) and 13 (1697). A single colony from each plate was grown overnight in LB supplemented with 4CP, miniprepped, and digestion mapped (BsaI). Gel electrophoresis results are shown in FIG. 14. Predicted bands for each expected species 1-4 are shown in Table 1, below:

TABLE 1
Predicted bands for each digestion species

| Lane | | |
|---|---|---|
| Number | Species | Band Sizes |
| 1 | 1696 | 6427 bp; 4593 bp; 1673 bp |
| 2 | 697 | 6225 bp; 5288 bp; 1673 bp |
| 3 | Template plasmid | 5166 bp; 855 bp |
| 4 | Circular DNA vector | 855 bp; 90 bp |

For colonies harboring either type of BAC (1696 or 1697), addition of either inducer appeared to increase GFP expressing colonies, indicating that expression of Bxb1 occurred in the absence of inducing agent. In fact, Bxb1 recombination had already occurred after transformation of template plasmid and plating on Cm/Kan plate. In 1606 BAC containing bacteria, non-induced Bxb1 resulted in >90% green colonies, indicating that more than 90% contained circular DNA vector with little or no template plasmid.

### Example 7. Production of circular DNA vectors containing therapeutic transgenes

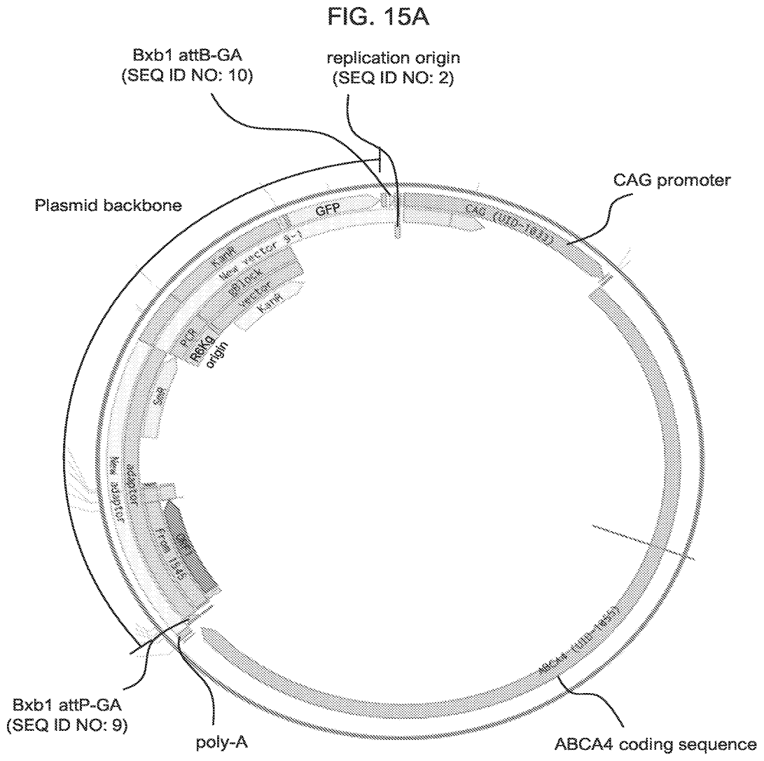

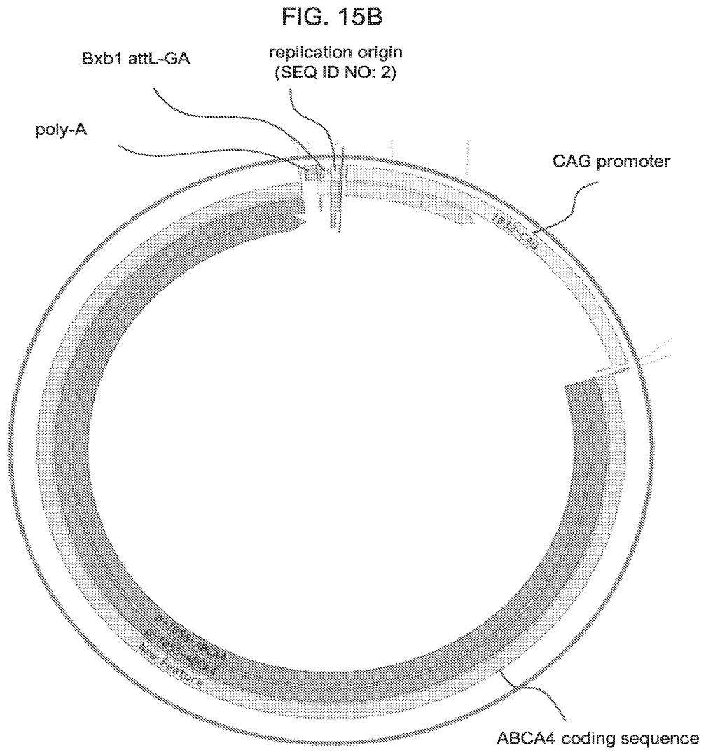

In this study, circular DNA vectors were made to include various types of therapeutic transgenes: (1) ABCA4, (2) IL-12, and (3) a tri-cistronic cassette encoding Flt3L, IL-12, and XCL1. ABCA4 and IL-12 constructs included a CAG promoter, and the tricistronic construct included CAG promoters upstream of each of the three genes. Exemplary sequences for the ABCA4 template plasmid and resulting circular DNA vector are given by FIG. 15A (SEQ ID NO: 7) and FIG. 15B (SEQ ID) NO: 8), respectively.

First, S1037 cells were transformed with 1696 BAC and grown overnight. After one day, cells were made competent and transformed with template plasmid encoding ABCA4, IL-12, or tricistronic cassette. Cells were plated on LB agar plate supplemented with Kan. After a three-day culture, small white colonies were picked (leaving red colonies) and grown overnight in LB+4CP for counterselection before purification.

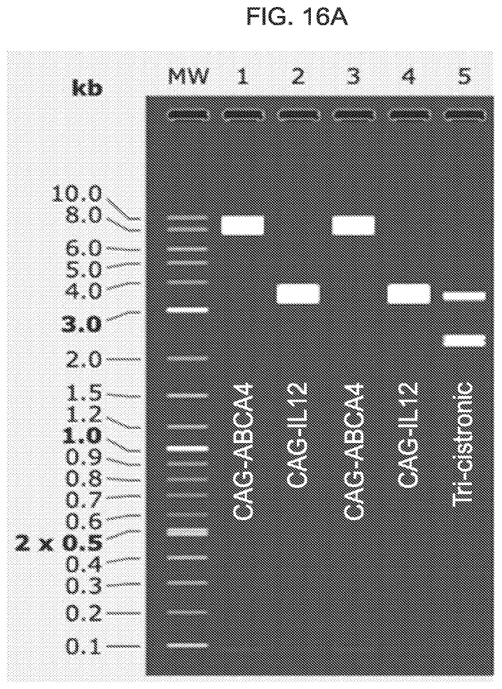

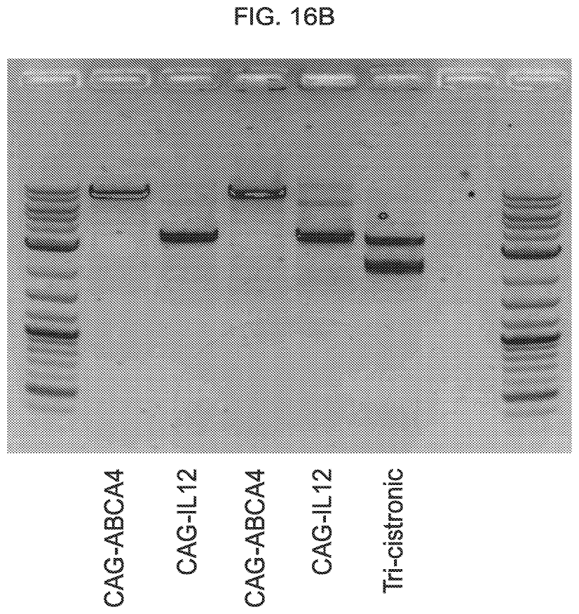

Purified constructs were screened from a single colony in each group using digestion mapping with BsaI. All colonies yielded circular DNA vector bands of the expected sizes, as shown in FIGS. 16A (theoretical gel profile) and 16B (actual gel profile). These results demonstrate that the circular DNA vector production process described herein can be broadly utilized to efficiently produce circular DNA vectors having transgenes of various sizes and configurations (e.g., multicistronic).

### Example 8. Stability of Circular DNA Vector Over Bacterial Growth and Scale-Up

A meaningful advantage of the vector system described herein is the ability to grow bacterial cells harboring circular DNA vectors without selection markers (e.g., after selectable markers and other bacterial backbone elements have been removed from culture). Toward this end, Applicant tested whether circular DNA vectors containing a therapeutic transgene could be stably expressed in bacterial culture over the many cell divisions associated with scaled up vector production.

Cells identified as containing ABCA4-encoding circular DNA vector without backbone, as in Example 7, were cultured overnight (14-16 hours each) over seven sequential nights. After the seventh culture, presence of circular DNA vector was confirmed by sequencing. Based on an average rate of three divisions per hour, this culture had undergone at least 294 divisions, and, surprisingly, had maintained expression of circular DNA vector despite the absence of selection.

Leveraging this remarkable stability, cells were scaled up to produce a larger quantity of circular DNA vector containing an ABCA4 transgene. Cells were re-streaked on LB plates and grown to produce a 2.5 L prep. A glycerol stock was produced, and this stock was re-streaked to produce a 25 L prep. Circular DNA vectors were purified from this prep, which yielded 18 mg of circular DNA vectors.

### Example 9. Detection of Monomeric Circular DNA Vectors

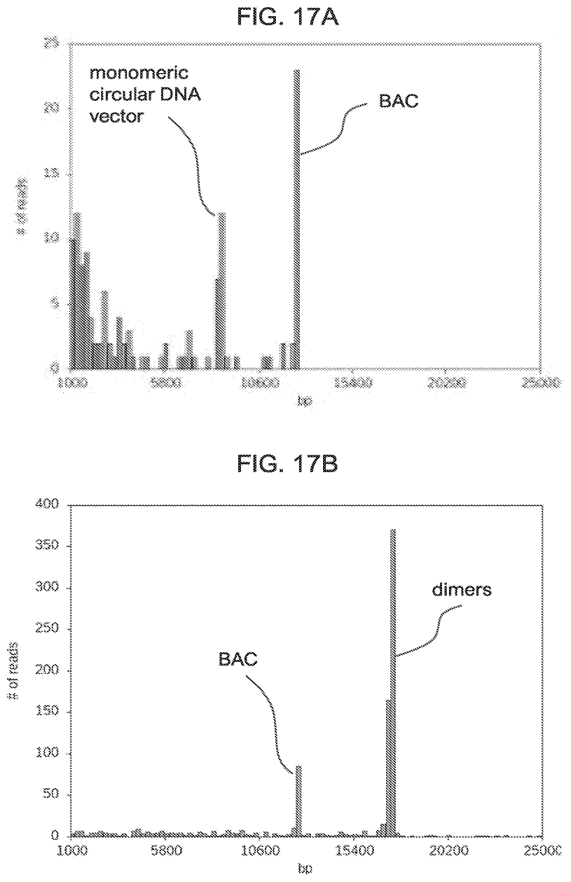

In this study, circular DNA vectors containing the ABCA4 transgene produced using 1696 BAC were sequenced to confirm that circular DNA vector is in monomeric form (as opposed to dimeric, which can result from recombination in trans with another template plasmid). In this study, S1037 bacteria were cultured in LB broth containing 25 μg/mL. Kan at 37C for two hours. Next, cells were transferred to a plate containing 4CP and 10 ng of DNA and incubated overnight at 37C. Samples were analyzed by long read sequencing using conventional methods (Oxford Nanopore). As shown in FIG. 17A, a monomer peak was observed, and no dimers we observed. In contrast, when cultures were incubated with Kan overnight, circular DNA vectors were primarily dimeric (FIG. 17B).

### Example 10. Circular DNA Vectors Made Using Bxb1 Helper Plasmid

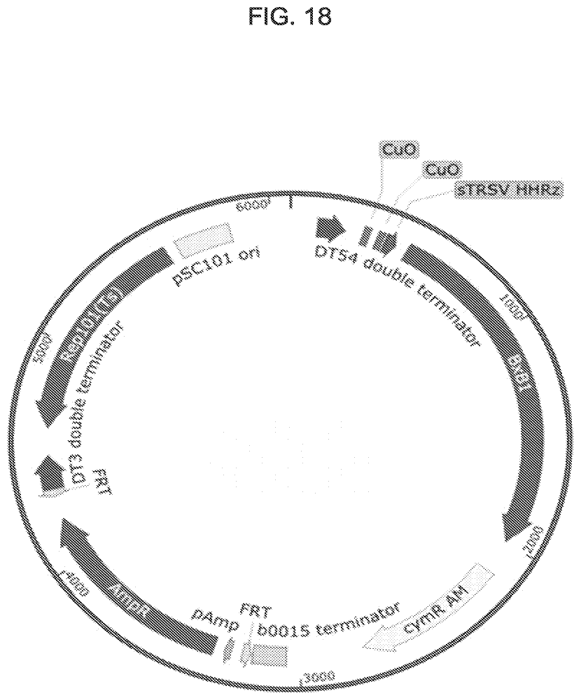

As an alternative strategy to BAC-Bxb1, circular DNA vectors were made using Bxb1 transformed into host cells using helper plasmids. An exemplary helper plasmid is shown in FIG. 18, which includes a cumate inducible promoter (CuO). Additionally, the helper plasmid included a temperature sensitive backbone to allow for removal of the helper plasmid following production of the circular DNA vector. The DNA sequence of this helper plasmid is given by SEQ ID NO: 11. Host cells used in this method were the same as in similar examples—S1037 cells having Rep (SEQ ID NO: 1) integrated in the host genome and driven by a constitutive promoter. Template plasmid was the same as in Example 6 (FIG. 7D).

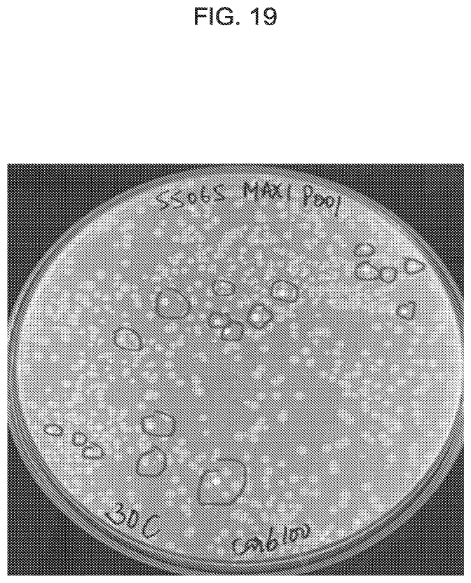

In this study, helper plasmid was transformed into S1037 cells and incubated with 100 μg/mL carbenicillin (carb100) overnight. Then plasmid template was transformed and plated with carb100 and 500 μM Cuma, and cells were grown at 30 C. Using this helper plasmid approach, several green colonies were observed (FIG. 19), indicating successful production of cells containing circular DNA vector without backbone byproducts.

### Example 11. Circular DNA Vectors Made by Integrating Bxb1 into the Host Genome

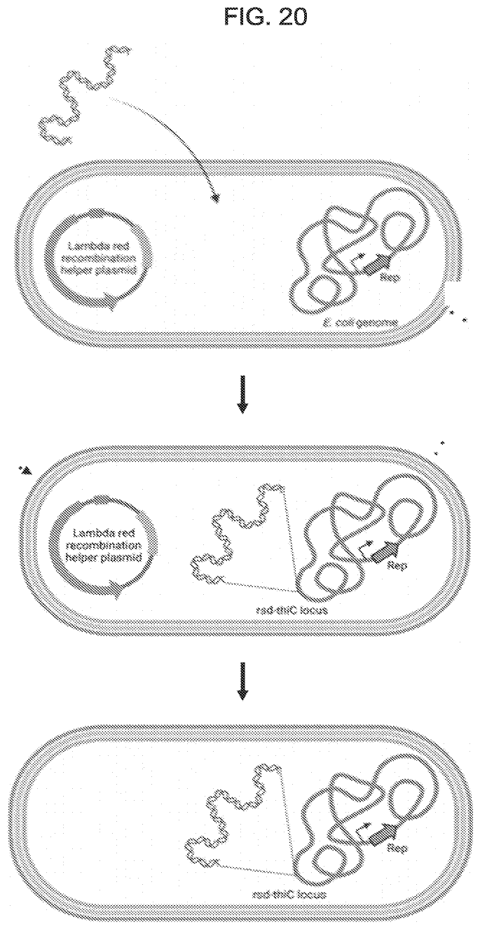

Another source of recombinase provided herein is via integration into the bacterial host genome. In this example, integration of Bxb1 was performed following the process illustrated in FIG. 20. First, S1037 cells harboring lambda red recombination helper plasmid were grown with 0.2% arabinose and made electrocompetent. Then 500 ng of linearized 1696 BAC was electroporated. After BAC 1696 was integrated into rsd-thiC locus, the lambda red recombination plasmid was removed.

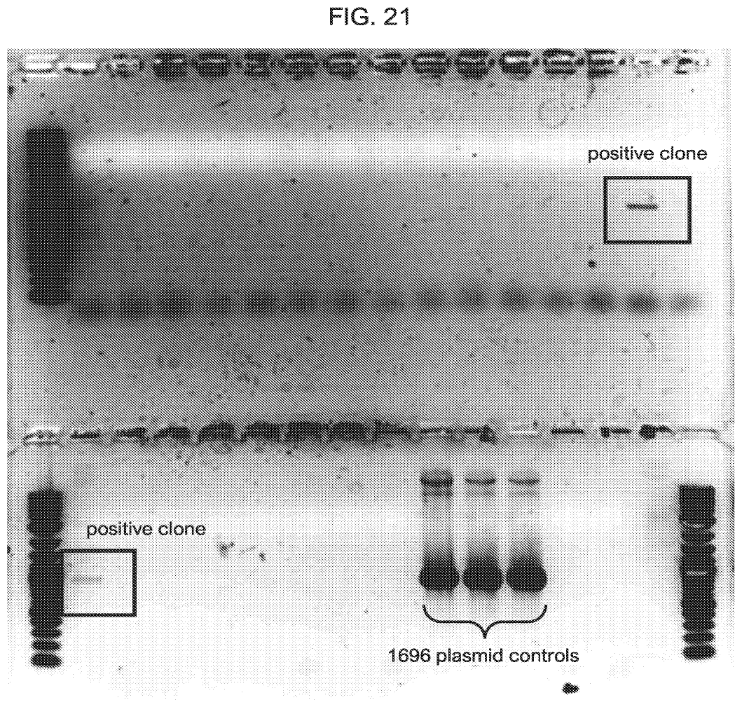

Colony PCR was performed on the resulting colonies, and positive clones were identified using gel electrophoresis (FIG. 21). Resulting cells are engineered to express both Rep and Bxb1 by genomic integration.

### Example 12. ABCA4 Protein Expression by Circular DNA Vectors Having a ColE2-P9 Ori

To determine whether bacterially produced circular DNA vectors having a ColE2-derived origin of replication are capable of expressing protein in human cells, an in vitro study was performed in which such circular DNA vectors carrying a human ABCA4 gene were transfected into HEK293T cells, and ABCA4 protein expression was assessed by western blot.

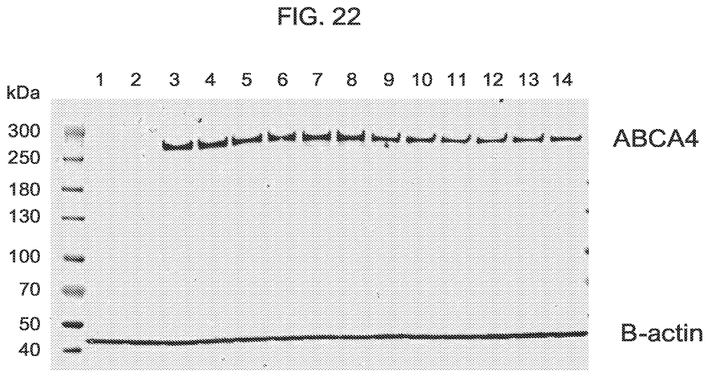

HEK293T cells were seeded in 24 well plates at 150,000 cells in 0.5 mL of standard media. Plates were incubated for 24 hours at 37C. At time of transfection, cells were 60-80% confluent. Cells were transfected with circular DNA vectors using Lipofectamine 3000 (Invitrogen) following the manufacturer's protocol. The total amount of DNA added per well was 500 ng. After 24 hours of incubation at 37C, cells were harvested for analysis by western blot, using beta actin as a control. Western blot results are shown in FIG. 22, and each lane is identified in Table 2, below.

TABLE 2
Sample identification for protein expression assay

| Lanes | Sample |
|---|---|
| 1 & 2 | Lipofectamine only (negative control) |
| 3 & 4 | Synthetic circular DNA (positive control) |
| 5-14 | Bacterially derived circular DNA |

These results show that bacterially produced circular DNA vectors having a ColE2-derived origin expressed their ABCA4 transgene in HEK293T human cells.

Sequence Listing

| | |
|---|---|
| SEQ ID NO: 1 | MSAVLQRFREKLPHKPYCTNDFAYGVRILPKNIAILARFIQQNQPHALYWLPFDVDRTG |
| ColE2-P9 | ASIDWSDRNCPAPNITVKNPRNGHAHLLYALALPVRTAPDASASALRYAAAIERALCE |
| replication | KLGADVNYSGLICKNPCHPEWQEVEWREEPYTLDELADYLDLSASARRSVDKNYGLG |
| protein | RNYHLFEKVRKWAYRAIRQGWPVFSQWLDAVIQRVEMYNASLPVPLSPAECRAIGKSI |
| | AKYTHRKFSPEGFSAVQAARGRKGGTKSKRAAVPTSARSLKPWEALGISRATYYRKL |
| | KCDPDLAK |
| SEQ ID NO: 2 | AGGGCGCTGTTATCTGATAAGGCTTATCTGGTCTCATTTT |
| ColE2-P9 | |
| origin of | |
| replication | |
| (40-bp) | |
| SEQ ID NO: 3 | AGGGCGCTGTTATCTGATAAGGCTTATCTGGTCTCA |
| ColE2-P9 | |
| origin of | |
| replication | |
| (36-bp) | |
| SEQ ID NO: 4 | GCGCTGTTATCTGATAAGGCTTATCTGGTCTCA |
| ColE2-P9 | |
| origin of | |
| replication | |
| (33-bp) | |
| SEQ ID NO: | gcggccgcaaggggttcgcgtcagcgggtgttggcgggtgtcggggctggcttaactatgcggcatcaga |
| 5: BAC1696 | gcagattgtactgagagtgcaccatatgcggtgtgaaataccgcacagatgcgtaaggagaaaataccgcat |
| | caggcgccattcgccattcagctgcgcaactgttgggaagggcgatcggtgcgggcctcttcgctattacgccagctggcga |
| | sagggggatgtgctgcaaggcgattaagttgggtaacgccagggttttcccagtcacgacgttgtaaa |
| | acgacggccagtgaattgtaatacgactcactatagggcgaattcGGTCTCAAGGCGC |
| | TGGAAGCGCGCTTTGTGCTGGAAGATAAGCTGATTCTGCTGGTGCTTGACGCCGCC |
| | CGCGTCAAACATCCTGCTTGAGTTCTGCGCTGTTAACGGGAAACACAGAAAAAAGC |
| | CCGCACCTGACAGTGCGGGCTTTTTTTTTCGACCAAAGGCTCGGTACCAAATTCCA |
| | GAAAAGACACCCGAAAGGGTGTTTTTTCGTTTTGGTCCcaattattgaaggcctccctaacggggggc |
| | ctttttttgtttctggtcAcccgcttaacgatcgttggctgaacaaacagacaatctggtctgtttgt |
| | attatggaaaatttttctgtataatagattcaacaaacagacaatctggtctgtttgtattatagctgt |
| | caccggatgtgctttcccggtctgatgagtccgtgaggacgaaacagcctctaaaataattttgtttaa |
| | TACTAGAGAAAGAGGAGAAATACTAGATGCGTGCACTGGTTGTTATT |
| | CGTCTGAGCCGTGTTACCGATGCAACCACCAGTCCGGAACGTCAGCTGGAAAGCTG |
| | TCAGCAGCTGTGTGCACAGCGTGGTTGGGATGTTGTTGGTGTTGCCGAAGATCTGG |
| | ATGTTAGCGGTGCAGTTGATCCGTTTGATCGTAAACGTCGTCCGAATCTGGCACGT |
| | TGGCTGGCATTTGAAGAACAGCCGTTTGATGTTATTGTTGCCTATCGTGTTGATCGT |
| | CTGACCCGTAGCATTCGTCATCTGCAGCAGCTGGTTCATTGGGCAGAAGATCATAA |
| | AAAGCTGGTTGTGAGCGCAACCGAAGCACATTTTGATACCACCACACCGTTTGCAG |
| | CAGTTGTTATTGCACTGATGGGCACCGTTGCACAGATGGAACTGGAAGCAATTAAA |
| | GAACGTAATCGTAGCGCAGCCCATTTTAACATTCGTGCAGGTAAATATCGTGGTAG |
| | CCTGCCTCCGTGGGGTTATCTGCCGACACGTGTTGATGGTGAATGGCGTCTGGTTCC |
| | GGATCCTGTTCAGCGTGAACGTATTCTGGAAGTTTATCATCGTGTGGTGGATAATC |
| | ATGAACCGCTGCATCTGGTTGCACATGATCTGAATCGTCGTGGTGTTCTGAGCCCG |
| | AAAGATTATTTTGCACAGCTGCAGGGTCGTGAACCGCAGGGTCGCGAATGGTCAGC |
| | AACCGCACTGAAACGTAGCATGATTAGCGAAGCAATGCTGGGTTATGCAACCCTG |
| | AATGGTAAAACCGTTCGTGATGATGATGGTGCACCGCTGGTTCGTGCAGAACCGAT |
| | TCTGACACGTGAACAGCTGGAAGCACTGCGTGCCGAACTGGTTAAAACCAGCCGT |
| | GCAAAACCGGCAGTTAGCACCCCGAGCCTGCTGCTGCGTGTTCTGTTTTGTGCAGT |
| | TTGTGGTGAACCGGCATACAAATTTGCCGGTGGTGGTCGTAAACATCCGCGTTATC |
| | GTTGTCGTAGCATGGGTTTTCCGAAACATTGTGGTAATGGTACAGTTGCAATGGCA |
| | GAATGGGATGCATTTTGCGAAGAACAGGTTCTGGATCTGCTGGGTGATGCCGAACG |
| | TCTGGAAAAAGTTTGGGTTGCAGGTAGCGATAGCGCAGTTGAACTGGCCGAAGTTA |
| | ATGCAGAACTGGTTGATCTGACCAGCCTGATTGGTAGTCCGGCATATCGTGCCGGT |
| | AGTCCGCAGCGTGAAGCACTGGATGCACGTATTGCAGCACTGGCAGCACGTCAAG |
| | AAGAATTAGAAGGTCTGGAAGCCCGTCCGAGCGGTTGGGAATGGCGTGAAACCGG |
| | TCAGCGTTTTGGTGATTGGTGGCGTGAGCAGGATACCGCAGCCAAAAATACCTGGC |
| | TGCGTAGTATGAATGTTCGCCTGACCTTTGATGTTCGCGGTGGTCTGACGCGTACCA |
| | TTGATTTTGGCGATCTGCAAGAATATGAACAGCATCTGCGTCTGGGTAGCGTTGTT |
| | GAACGTCTGCATACCGGCATGAGCTAActcggtaccaaattccagaaaagaggcctcccgaaaggggggcct |
| | tttttcgttttggtccaatggcggcgcgccatcgaatggtgcaaaacctttcgcggtatggcatgatagcgc |
| | ccggaagagagtcaattcagggtggtgaatatgagcccgaaacgtcgtacccaggcagaacgtgcaatgga |
| | aacccagggtaaactgattgcagcagcactgggtgttctgcgtgaaaaaggttatgcaggttttcgtattgc |
| | agatgttccgggtgcagccggtgttagccgtggtgcacagagccatcattttccgaccaaactggaactg |
| | ctgctggcaacctttgaatggctgtatgagcagattaccgaacgtagccgtgcacgtctggcaaaactga |
| | aaccggaagatgatgttattcagcagatgctggatgatgcagcagaattttttctggatgatgattttagca |
| | tcggcctggatctgattgttgcagcagatcgtgatccggcactgcgtgaaggtattcagcgtaccgttgaacg |
| | taatcgttttgttgttgaagatatgtggctgggtgtgctggtgagccgtggtctgagccgtgatgatgccgaa |
| | gatattctgtggctgatttttaacagcgttcgtggtctggtagttcgtagcctgtggcagaaagataaaga |
| | acgttttgaacgtgtgcgtaatagcaccctggaaattgcacgtgaacgttatgcaaaattcaaacgttgataa |
| | ggatcctaattggtaacgaatcagacaattgacggctcgagggagtagcatagggtttgcagaatccctgctt |
| | cgtccatttgacaggcacattatgcatcgatgataagctgtcaaacatgagcagatcctctacgccggacgca |
| | tcgtggccggcatcaccggcgccacaggtgcggttgctggcgcctatatcgccgacatcaccgatggggaag |
| | atcgggctcgccacttcgggctcatgagcaaatattttatctgaggtgcttcctcgctcactgactcgctgcac |
| | gaggcagacctcaGGATCTccaggcatcaaataaaacgaaaggctcagtcgaaagactgggcctttcgttttat |
| | ctgttgtttgtcggtgaacgctctctactagagtcacactggctcaccttcggggggcctttctgcgtttata |
| | GGATCTgcgaagttcctatactttctagagaataggaacttcgGATCTgtgcgcggaacccctatttgtttat |
| | ttttctaaatacattcaaatatgtatccgctcatgagacaataaccctgataaatgcttcaataatattgaaaa |
| | aggaagagtatgagtattcaacatttccgtgtcgcccttattcccttttttgcggcattttgccttcctgtttttg |
| | ctcacccagaaacgctggtgaaagtaaaagatgctgaagatcagttgggtgcacgagtgggttacatcgaactgga |
| | tctcaacagcggtaagatccttgagagttttcgccccgaagaacgttttccaatgatgagcacttttaaagttctg |
| | ctatgtggcgcggtattatcccgtattgacgccgggcaagagcaactcggtcgccgcatacactattctcagaatg |
| | acttggttgagtactcaccagtcacagaaaagcatcttacggatggcatgacagtaagagaattatgcagtgctgc |
| | cataaccatgagtgataacactgcggccaacttacttctgacaacgatcggaggaccgaaggagctaaccgctttt |
| | ttgcacaacatgggggatcatgtaactcgccttgatcgttgggaaccggagctgaatgaagccataccaaacgacga |
| | gcgtgacaccacgatgcctgtagcaatggcaacaacgttgcgcaaactattaactggcgaactacttactctagctt |
| | cccggcaacaattaatagactggatggaggcggataaagttgcaggaccacttctgcgctcggcccttccggctggc |
| | tggtttattgctgataaatctggagccggtgagcgtggttctcgcggtatcattgcagcactggggccagatggtaa |
| | gccctcccgtatcgtagttatctacacgacggggagtcaggcaactatggatgaacgaaatagacagatcgctgaga |
| | taggtgcctcactgattaagcattggtaactgtcagaccaagtttactcatatatactttagattgatttaaaactt |
| | catttttaatttaaaaggatctaggtgaagatcctttttgataatcGGATCTgcgaagttcctatactttctaga |
| | gaataggaacttcgCCGGCTTATCGGTCAGTTTCACCTGATTTACGTAAAAACCCGCTTCGG |
| | CGGGTTTTTGCTTTTGGAGGGGCAGAAAGATGAATGACTGTCCACGACGCTATACC |
| | CAAAAGAAAAAAAAAAAAaCCCGCCCCTGACAGGGCGGGGTTTTTTTTGCTTCAAA |
| | TGCGGATTTGACAGCTAGCTCAGTCCTAGGTATTGTGCTAGCCGTAATTTACATTCA |
| | ATGCCCCATTTGCGGGGCTAATTTCTTGTCGGAGTGCCTTAACTGGCTGAGACCaagc |
| | ttgagtattctatagtctcacctaaatagcttggcgtaatcatggtcatagctgtttcctgtgtgaaattgttatcc |
| | gctcacaattccacacaacatacgagccggaagcatasagtgtaaagcctggggtgcctaatgagtgagctaactca |
| | cattaattgcgttgcgctcactgcccgctttccagtcgggaaacctgtcgtgccagctgcattaatgaatcggccaa |
| | cgcgaaccccttgcggccgcccgggccgtcgaccaattctcatgtttgacagcttatcatcgaatttctgccattca |
| | tccgcttattatcacttattcaggcgtagcaaccaggcgtttaagggcaccaataactgccttaaaaaaattacgcc |
| | ccgccctgccactcatcgcagtactgttgtaattcattaagcattctgccgacatggaagccatcacaaacggcatga |
| | tgaacctgaatagccagcggcatcagcaccttgtcgccttgcgtataatatttgcccatggtgaaaacgggggcgaag |
| | aagttgtccatattggccacgtttaaatcaaaactggtgaaactcacccagggattggctgagacgaaaaacatattc |
| | tcaataaaccctttagggaaataggccaggttttcaccgtaacacgccacatcttgcgaatatatgtgtagaaactgc |
| | cggaaatcgtcgtggtattcactccagagcgatgaaaacgtttcagtttgctcatggaaaacggtgtaacaagggtga |
| | acactatcccatatcaccagctcaccgtctttcattgccatacgaaattccggatgagcattcatcagggggcaagaa |
| | tgtgaataaaggccggataaaacttgtgcttatttttctttacggtctttaaaaaggccgtaatatccagctgaacgg |
| | tctggttataggtacattgagcaactgactgaaatgcctcaaaatgttctttacgatgccattgggatatatcaacgg |
| | tggtatatccagtgatttttttctccattttagcttccttagctcctgaaaatctcgataactcaaaaaatacgcccg |
| | gtagtgatcttatttcattatggtgaaagttggaacctcttacgtgccgatcaacgtctcattttcgccaaaagttgg |
| | cccagggcttcccggtatcaacagggacaccaggatttatttattctgcgaagtgatcttccgtcacaggtatttatt |
| | cgcgataagctcatggagcggcgtaaccgtcgcacaggaaggacagagaaagcgcggatctgggaagtgacggacaga |
| | acggtcaggacctggattggggaggcggttgccgccgctgctgctgacggtgtgacgttctctgttccggtcacacca |
| | catacgttccgccattcctatgcgatgcacatgctgtatgccggtataccgctgaaagttctgcaaagcctgatggga |
| | cataagtccatcagttcaacggaagtctacacgaaggtttttgcgctggatgtggctgcccggcaccgggtgcagttt |
| | gcgatgccggagtctgatgcggttgcgatgctgaaacaattatcctgagaataaatgccttggcctttatatggaaat |
| | gtggaactgagtggatatgctgtttttgtctgttaaacagagaagctggctgttatccactgagaagcgaacgaaaca |
| | gtcgggaaaatctcccattatcgtagagatccgcattattaatctcaggagcctgtgtagcgtttataggaagtagtg |
| | ttctgtcatgatgcctgcaagcggtaacgaaaacgatttgaatatgccttcaggaacaatagaaatcttcgtgcggtg |
| | ttacgttgaagtggagcggattatgtcagcaatggacagaacaacctaatgaacacagaaccatgatgtggtctgtcc |
| | ttttacagccagtagtgctcgccgcagtcgagcgacagggcgaagccctcggctggttgccctcgccgctgggctggc |
| | ggccgtctatggccctgcaaacgcgccagaaacgccgtcgaagccgtgtgcgagacaccgcggccggccgccggcgtt |
| | gtggatacctcgcggaaaacttggccctcactgacagatgaggggcggacgttgacacttgaggggccgactcacccg |
| | gcgcggcgttgacagatgaggggcaggctcgatttcggccggcgacgtggagctggccagcctcgcaaatcggcgaaa |
| | acgcctgattttacgcgagtttcccacagatgatgtggacaagcctggggataagtgccctgcggtattgacacttga |
| | ggggcgcgactactgacagatgaggggcgcgatccttgacacttgaggggcagagtgctgacagatgagggggcacct |
| | attgacatttgaggggctgtccacaggcagaaaatccagcatttgcaagggtttccgcccgtttttcggccaccgcta |
| | acctgtcttttaacctgcttttaaaccaatatttataaaccttgtttttaaccagggctgcgccctgtgcgcgtgac |
| | cgcgcacgccgaaggggggtgcccccccttctcgaaccctcccggtcgagtgagcgaggaagcaccagggaacagcac |
| | ttatatattctgcttacacacgatgcctgaaaaaacttcccttggggttatccacttatccacggggatatttttata |
| | attattttttttatagtttttagatcttcttttttagagcgccttgtaggcctttatccatgctggttctagagaagg |
| | tgttgtgacaaattgccctttcagtgtgacaaatcaccctcaaatgacagtcctgtctgtgacaaattgcccttaacc |
| | ctgtgacaaattgccctcagaagaagctgttttttcacaaagttatccctgcttattgactcttttttatttagtgtg |
| | acaatctaaaaacttgtcacacttcacatggatctgtcatggcggaaacagcggttatcaatcacaagaaacgtaaaa |
| | atagcccgcgaatcgtccagtcaaacgacctcactgaggcggcatatagtctctcccgggatcaaaaacgtatgctgt |
| | atctgttcgttgaccagatcagaaaatctgatggcaccctacaggaacatgacggtatctgcgagatccatgttgcta |
| | aatatgctgaaatattcggattgacctctgcggaagccagtaaggatatacggcaggcattgaagagtttcgggggaa |
| | ggaagtggttttttatcgccctgaagaggatgccggcgatgaaaaaggctatgaatcttttccttggtttatcaaacg |
| | tgcgcacagtccatccagagggctttacagtgtacatatcaacccatatctcattcccttctttatcgggttacagaa |
| | ccggtttacgcagtttcggcttagtgaaacaaaagaaatcaccaatccgtatgccatgcgtttatacgaatccctgtgt |
| | cagtatcgtaagccggatggctcaggcatcgtctctctgaaaatcgactggatcatagagcgttaccagctgcctcaaa |
| | gttaccagcgtatgcctgacttccgccgccgcttcctgcaggtctgtgttaatgagatcaacagcagaactccaatgcg |
| | cctctcatacattgagaaaaagaaaggccgccagacgactcatatgtattttccttccgcgatatcacttccatgacga |
| | caggatagtctgagggttatctgtcacagatttgagggtggttcgtcacatttgttctgacctactgagggtaatttgt |
| | cacagttttgctgtttccttcagcctgcatggattttctcatactttttgaactgtaattuttaaggaagccaaatttg |
| | agggcagtttgtcacagttgatttccttctctttcccttcgtcatgtgacctgatatcgggggttagttcgtcatca |
| | ttgatgagggttgattatcacagtttattactctgaattggctatccgcgtgtgtacctctacctggagtttttcccac |
| | ggtggatatttcttcttgcgctgagcgtaagagctatctgacagaacagttcttctttgcttcctcgcsagttcgctcg |
| | ctatgctcggttacacggctgcggcgagcgctagtgataataagtgactgaggtatgtgctcttcttatctccttttgt |
| | agtgttgctcttattttaaacaactttgcggttttttgatgactttgcgattttgttgttgctttgcagtaaattgcaa |
| | gatttaataaaaaaacgcaaagcaatgattaaaggatgttcagaatgaaactcatggaaacacttaaccagtgcataaa |
| | cgctggtcatgaaatgacgaaggctatcgccattgcacagtttaatgatgacagcccggaagcgaggaaaataacccg |
| | gcgctggagaataggtgaagcagcggatttagttggggtttcttctcaggctatcagagatgccgagaaagcagggcgac |
| | taccgcacccggatatggaaattcgaggacgggttgagcaacgtgttggttatacaattgaacaaattaatcatatgcgt |
| | gatgtgtttggtacgcgattgcgacgtgctgaagacgtatttccaccggtgatcggggttgctgcccataaaggtggcgt |
| | ttacaaaacctcagtttctgttcatcttgctcaggatctggctctgaaggggctacgtgttttgctcgtggaaggtaacg |
| | acccccagggaacagcctcaatgtatcacggatgggtaccagatcttcatattcatgcagaagacactctcctgcctttc |
| | tatcttggggaaaaggacgatgtcacttatgcaataaagcccacttgctggccggggcttgacattattccttcctgtct |
| | ggctctgcaccgtattgaaactgagttaatgggcaaatttgatgaaggtaaactgcccaccgatccacacctgatgctcc |
| | gactggccattgaaactgttgctcatgactatgatgtcatagttattgacagcgcgcctaacctgggtatcggcacgatt |
| | aatgtcgtatgtgctgctgatgtgctgattgttcccacgcctgctgagttgtttgactacacctccgcactgcagttttt |
| | cgatatgcttcgtgatctgctcaagaacgttgatcttaaagggttcgagcctgatgtacgtattttgcttaccaaataca |
| | gcaatagtaatggctctcagtccccgtggatggaggagcaaattcgggatgcctggggaagcatggttctaaaaaatgtt |
| | gtacgtgaaacggatgaagttggtaaaggtcagatccggatgagaactgtttttgaacaggccattgatcaacgctcttc |
| | aactggtgcctggagaaatgctctttctatttgggaacctgtctgcaatgaaattttcgatcgtctgattaaaccacgct |
| | gggagattagataatgaagcgtgcgcctgttattccaaaacatacgctcaatactcaaccggttgaagatacttcgttat |
| | cgacaccagctgccccgatggtggattcgttaattgcgcgcgtaggagtaatggctcgcggtaatgccattactttgcct |
| | gtatgtggtcgggatgtgaagtttactcttgaagtgctccggggtgatagtgttgagaagacctctcgggtatggtcagg |
| | taatgaacgtgaccaggagctgcttactgaggacgcactggatgatctcatcccttcttttctactgactggtcaacaga |
| | caccggcgttcggtcgaagagtatctggtgtcatagaaattgccgatgggagtcgccgtcgtaaagctgctgcacttacc |
| | gaaagtgattatcgtgttctggttggcgagctggatgatgagcagatggctgcattatccagattgggtaacgattatcg |
| | cccaacaagtgcttatgaacgtggtcagcgttatgcaagccgattgcagaatgaatttgctggaaatatttctgcgctgg |
| | ctgatgcggaaaatatttcacgtaagattattacccgctgtatcaacaccgccaaattgcctaaatcagttgttgctctt |
| | ttttctcaccccggtgaactatctgcccggtcaggtgatgcacttcaaaaagcctttacagataaagaggaattacttaa |
| | gcagcaggcatctaaccttcatgagcagaaaaaagctggggtgatatttgaagctgaagaagttatcactcttttaactt |
| | ctgtgcttaaaacgtcatctgcatcaagaactagtttaagctcacgacatcagtttgctcctggagcgacagtattgtat |
| | aagggcgataaaatggtgcttaacctggacaggtctcgtgttccaactgagtgtatagagaaaattgaggccattcttaa |
| | ggaacttgaaaagccagcaccctgatgcgaccacgttttagtctacgtttatctgtctttacttaatgtcctttgttaca |
| | ggccagaaagcataactggcctgaatattctctctgggcccactgttccacttgtatcgtcggtctgataatcagactgg |
| | gaccacggtcccactcgtatcgtcggtctgattattagtctgggaccacggtcccactcgtatcgtcggtctgattatta |
| | gtctgggaccacggtcccactcgtatcgtcggtctgataatcagactgggaccacggtcccactcgtatcgtcggtctga |
| | ttattagtctgggaccatggtcccactcgtatcgtcggtctgattattagtctgggaccacggtcccactcgtatcgtcg |
| | gtctgattattagtctggaaccacggtcccactcgtatcgtcggtctgattattagtctgggaccacggtcccactcgta |
| | tcgtcggtctgattattagtctgggaccacgatcccactcgtgttgtcggtctgattatcggtctgggaccacggtccca |
| | cttgtattgtcgatcagactatcagcgtgagactacgattccatcaatgcctgtcaagggcaagtattgacatgtcgtcg |
| | taacctgtagaacggagtaacctcggtgtgcggttgtatgcctgctgtggattgctgctgtgtcctgcttatccacaaca |
| | ttttgcgcacggttatgtggacaaaatacctggttacccaggccgtgccggcacgttaaccgggctgcatccgatgcaag |
| | tgtgtcgctgtcgacgagctcgcgagctcggacatgaggttgccccgtattcagtgtcgctgatttgtattgtctgaagt |
| | tgtttttacgttaagttgatgcagatcaattaatacgatacctgcgtcataattgattatttgacgtggtttgatggcct |
| | ccacgcacgttgtgatatgtagatgataatcattatcactttacgggtcctttccggtgatccgacaggttacggggcgg |
| | cgacctcgcgggttttcgctatttatgaaaattttccggtttaaggcgtttccgttcttcttcgtcataacttaatgttt |
| | ttatttaaaataccctctgaaaagaaaggaaacgacaggtgctgaaagcgagctttttggcctctgtcgtttcctttctc |
| | tgtttttgtccgtggaatgaacaatggaagtccgagctcatcgctaataacttcgtatagcatacattatacgaagttat |
| | attcgat |
| SEQ ID NO: | GCGGCCGCAAGGGGTTCGCGTCAGCGGGTGTTGGCGGGTGTCGGGGCTGGCTTAA |
| 6: BAC1697 | CTATGCGGCATCAGAGCAGATTGTACTGAGAGTGCACCATATGCGGTGTGAAATAC |
| | CGCACAGATGCGTAAGGAGAAAATACCGCATCAGGCGCCATTCGCCATTCAGCTG |
| | CGCAACTGTTGGGAAGGGCGATCGGTGCGGGCCTCTTCGCTATTACGCCAGCTGGC |
| | GAAAGGGGGATGTGCTGCAAGGCGATTAAGTTGGGTAACGCCAGGGTTTTCCCAG |
| | TCACGACGTTGTAAAACGACGGCCAGTGAATTGTAATACGACTCACTATAGGGCGA |
| | ATTCGGTCTCAACCAATTGGCGCGCGCTTCGCAATAAAATCTCCCTTCGCCCAGTAT |
| | TGGGAAAAGTAGATACATTCAAACTGTGTACGCTGTTTCGTCTCACAGTTCACGAC |
| | ATTTAATAAAAAAAGGGGGTCGCAAGATCGCCCTTTTTTACGTATGACACAGTGA |
| | AAAATGGCGCCCATCGGCGCCATTTTTTTATGcaattattgaaggcctccctaacggggggcctttttttgttt |
| | ctggtcAcccgcttaacgatcgttggctgagasaccaattgtccatattgcatcagacattgccgtcactgcgtct |
| | tttactggctcttctcgctaaccaaaccggtaaccccgcttattaaaagcattctgtaacaaagcgggaccaaagc |
| | catgacaaaaacgcgtaacasaagtgtctataatcacggcagaasagtccacattgattatttgcacggcgtcaca |
| | ctttgctatgccatagcatttttatccataagattagcggatcctacctgacgctttttatcgcaactctctactg |
| | tttctccatTcccgagctgtcaccggatgtgctttccggtctgatgagtccgtgaggacgaaacagcct |
| | ctacaaataattttgtttaaTACTAGAGAAAGAGGAGAAATACTAGATGCGTGCACTGGTTGTTA |
| | TTCGTCTGAGCCGTGTTACCGATGCAACCACCAGTCCGGAACGTCAGCTGGAAAGC |
| | TGTCAGCAGCTGTGTGCACAGCGTGGTTGGGATGTTGTTGGTGTTGCCGAAGATCT |
| | GGATGTTAGCGGTGCAGTTGATCCGTTTGATCGTAAACGTCGTCCGAATCTGGCAC |
| | GTTGGCTGGCATTTGAAGAACAGCCGTTTGATGTTATTGTTGCCTATCGTGTTGATC |
| | GTCTGACCCGTAGCATTCGTCATCTGCAGCAGCTGGTTCATTGGGCAGAAGATCAT |
| | AAAAAGCTGGTTGTGAGCGCAACCGAAGCACATTTTGATACCACCACACCGTTTGC |
| | AGCAGTTGTTATTGCACTGATGGGCACCGTTGCACAGATGGAACTGGAAGCAATTA |
| | AAGAACGTAATCGTAGCGCAGCCCATTTTAACATTCGTGCAGGTAAATATCGTGGT |
| | AGCCTGCCTCCGTGGGGTTATCTGCCGACACGTGTTGATGGTGAATGGCGTCTGGT |
| | TCCGGATCCTGTTCAGCGTGAACGTATTCTGGAAGTTTATCATCGTGTGGTGGATA |
| | ATCATGAACCGCTGCATCTGGTTGCACATGATCTGAATCGTCGTGGTGTTCTGAGC |
| | CCGAAAGATTATTTTGCACAGCTGCAGGGTCGTGAACCGCAGGGTCGCGAATGGTC |
| | AGCAACCGCACTGAAACGTAGCATGATTAGCGAAGCAATGCTGGGTTATGCAACC |
| | CTGAATGGTAAAACCGTTCGTGATGATGATGGTGCACCGCTGGTTCGTGCAGAACC |
| | GATTCTGACACGTGAACAGCTGGAAGCACTGCGTGCCGAACTGGTTAAAACCAGC |
| | CGTGCAAAACCGGCAGTTAGCACCCCGAGCCTGCTGCTGCGTGTTCTGTTTTGTGC |
| | AGTTTGTGGTGAACCGGCATACAAATTTGCCGGTGGTGGTCGTAAACATCCGCGTT |
| | ATCGTTGTCGTAGCATGGGTTTTCCGAAACATTGTGGTAATGGTACAGTTGCAATG |
| | GCAGAATGGGATGCATTTTGCGAAGAACAGGTTCTGGATCTGCTGGGTGATGCCGA |
| | ACGTCTGGAAAAAGTTTGGGTTGCAGGTAGCGATAGCGCAGTTGAACTGGCCGAA |
| | GTTAATGCAGAACTGGTTGATCTGACCAGCCTGATTGGTAGTCCGGCATATCGTGC |
| | CGGTAGTCCGCAGCGTGAAGCACTGGATGCACGTATTGCAGCACTGGCAGCACGTC |
| | AAGAAGAATTAGAAGGTCTGGAAGCCCGTCCGAGCGGTTGGGAATGGCGTGAAAC |
| | CGGTCAGCGTTTTGGTGATTGGTGGCGTGAGCAGGATACCGCAGCCAAAAATACCT |
| | GGCTGCGTAGTATGAATGTTCGCCTGACCTTTGATGTTCGCGGTGGTCTGACGCGT |
| | ACCATTGATTTTGGCGATCTGCAAGAATATGAACAGCATCTGCGTCTGGGTAGCGT |
| | TGTTGAACGTCTGCATACCGGCATGAGCTAActcggtaccaaattccagaaaagaggcctcccgaaaggg |
| | gggccttttttcgttttggtccaatggcggcgcgccatcgaatggtgcaaaacctttcgcggtatggcatgatag |
| | cgcccggaagagagtcaattcagggtggtgaatatggctgaagcgcaaaatgatcccctgctgccgggatactcg |
| | tttaatgcccatctggtggcgggtttaacgccgattgaggccaacggttatctcgatttttttatcgaccgaccg |
| | ctgggaatgaaaggttatattctcaatctcaccattcgcggtcagggggtggtgaaaaatcagggacgagaattt |
| | gtttgccgaccgggtgatattttgctgttcccgccaggagagattcatcactacggtcgtcatccggaggctcgc |
| | gaatggtatcaccagtgggtttactttcgtccgcgcgcctactggcatgaatggcttaactggccgtcaatattt |
| | gccaatacggggttctttcgcccggatgaagcgcaccagccgcatttcagcgacttttttgggcaaatcattaac |
| | gccgggcaaggggaagggcgctattcggagctgctggcgataaatctgcttgagcaattgttactgcggcgcatg |
| | ctagcgattaacggatcgctccatccaccgatggataatcgggtacgcgaggcttgtcagtacatcagcgatcac |
| | ctggcagacagcaattttgatatcgccagcgtcgcacagcatgtttgcttgtcgccgtcgcgtctgtcacatctt |
| | ttccgccagcagttagggattagcgtcttaagctggcgcgaggaccaacgtatcagccaggcgaagctgcttttga |
| | gcaccacccggatgcctatcgccaccgtcggtcgcaatgttggttttgacgatcaactctatttctcgcgggtatt |
| | taaaaaatgcaccggggccagcccgagcgagttccgtgccggtttggaagaaaaagtgaatgatgtagccgtcaag |
| | ttgtcatgataagatcctattccagcgggattaaagaggagcgattaagcatggttactatcaatacggaatctgc |
| | tttaacgccacgttctttgcgggatacgcggcgtatgaatatgtttgtttcggtagctgctgcggtcgcaggattg |
| | ttatttggtcttgatatcggcgtaatcgccggagcgttgccgttcattaccgatcactttgtgctgaccagtcgtt |
| | tgcaggaatgggggttagtagcatgatgctcggtgcagcaattggtgcgctgtttaatggttggctgtcgttccgc |
| | ctggggcgtaaatacagcctgatggcgggggccatcctgtttgtactcggttctatagggtccgcttttgcgacca |
| | gcgtagagatgttaatcgccgctcgtgtggtgctgggcattgctgtcgggatcgcgtcttacaccgctcctctgta |
| | tctttctgaaatggcaagtgaaaacgttcgcggtaagatgatcagtatgtaccagttgatggtcacactcggcatc |
| | gtgctggcgtttttatccgatacagcgttcagttatagcggtaactggcgcgcaatgttgggggttcttgctttac |
| | cagcagttctgctgattattctggtagtatttctgccaaatagcccgcgctggctggggaaaaggggcgtcatat |
| | tgaggcggaagaagtattgcgtatgctgcgcgatacgtcggaaaaagcgcgagaagaactcaacgaaattcgtga |
| | agcctgaagttaaaacagggcggttgggcactgtttaagatcaaccgtaacgtccgtcgtgctgtgtttctcggt |
| | atgttgttgcaggcgatgcagcagtttaccggtatgaacatcatcatgtactacgcgccgcgtatcttcaaaatg |
| | gcgggctttacgaccacagaacaacagatgattgcgactctggtcgtagggctgacctttatgttcgccaccttt |
| | attgcggtgtttacggtagataaagcagggcgtaaaccggctctgaaaattggtttcagcgtgatggcgttaggc |
| | actctggtgctgggctattgcctgatgcagtttgataacggtacggcttccagtggcttgtcctggctctctgtt |
| | ggcatgacgatgatgtgtattgccggttatgcgatgagcgccgcgccagtggtgtggatcctgtgctctgaaatt |
| | cagccgctgaaatgccgcgatttcggtattacctgttcgaccaccacgaactgggtgtcgaatatgattatcggc |
| | gcgaccttcctgacactgcttgatagcattggcgctgccggtacgttctggctctacactgcgctgaacattgcg |
| | tttgtgggcattactttctggctcattccggaaaccaaaaatgtcacgctggaacatatcgaacgcaaactgatg |
| | gcaggcgagaagttgagaaatatcggcgtctgataaggatcctaattggtaacgaatcagacaattgacggctcg |
| | agggagtagcatagggtttgcagaatccctgcttcgtccatttgacaggcacattatgcatcgatgataagctgt |
| | caaacatgagcagatcctctacgccggacgcatcgtggccggcatcaccggcgccacaggtgcggttgctggcgc |
| | ctatatcgccgacatcaccgatggggaagatcgggctcgccacttcgggctcatgagcaaatattttatctgagg |
| | tgcttcctcgctcactgactcgctgcacgaggcagacctcaGGATCTccaggcatcaaataaaacgaaaggctca |
| | gtcgaaagactgggcctttcgttttatctgttgtttgtcggtgaacgctctctactagagtcac |
| | actggctcaccttcgggtgggcctttctgcgtttataGGATCCGAAGTTCCTATTCTCTAGAAAGTATAGG |
| | AACTTCGGGTCCCCAATAATTACGATTTAAATTGGCGAAAATGAGACGTTGATCGG |
| | CACGTAAGAGGTTCCAACTTTCACCATAATGAAATAAGATCACTACCGGGCGTATT |
| | TTTTGAGTTATCGAGATTTTCAGGAGCTAAGGAAGCTAAAATGGAGAAAAAAATC |
| | ACTGGATATACCACCGTTGATATATCCCAATGGCATCGTAAAGAACATTTTGAGGC |
| | ATTTCAGTCAGTTGCTCAATGTACCTATAACCAGACCGTTCAGCTGGATATTACGG |
| | CCTTTTTAAAGACCGTAAAGAAAAATAAGCACAAGTTTTATCCGGCCTTTATTCAC |
| | ATTCTTGCCCGCCTGATGAATGCTCATCCGGAATTTCGTATGGCAATGAAAGACGG |
| | TGAGCTGGTGATATGGGATAGTGTTCACCCTTGTTACACCGTTTTCCATGAGCAAA |
| | CTGAAACGTTTTCATCGCTCTGGAGTGAATACCACGACGATTTCCGGCAGTTTCTAC |
| | ACATATATTCGCAAGATGTGGCGTGTTACGGTGAAAACCTGGCCTATTTCCCTAAA |
| | GGGTTTATTGAGAATATGTTTTTCGTCTCAGCCAATCCCTGGGTGAGTTTCACCAGT |
| | TTTGATTTAAACGTGGCCAATATGGACAACTTCTTCGCCCCCGTTTTCACCATGGGC |
| | AAATATTATACGCAAGGCGACAAGGTGCTGATGCCGCTGGCGATTCAGGTTCATCA |
| | TGCCGTTTGTGATGGCTTCCATGTCGGCAGAATGCTTAATGAATTACAACAGTACT |
| | GCGATGAGTGGCAGGGGGGGGCGTAAGAAGTTCCTATTCTCTAGAAAGTATAGGA |
| | ACTTCGCAGACAAAAAAAATGGCGCACAATGTGCGCCATTTTTCACTTCACAGGTA |
| | CTATTGTTTTGAATTGAAAAGGGCGCTTCGGCGCCCTTTTTGCATTTGTTGACGGCA |
| | TATATTTGTATATCGAAGCGCCCTGATGGGCGCTTTTTTTATTTAATCGATAACCAG |
| | AACATTGCTGGCCGATGAGCTGACCATTGGGCCTATCCGGGCTGTCCCGATGGATA |
| | TTACGCCGAAGTATGTGGGAATTGCCAGCGGATTGATGAACGCCGTGAGACCAAG |
| | CTTACGTCTCATTTTCGCCAAAAGTTGGCCCAGGGCTTCCCGGTATCAACAGGGAC |
| | ACCAGGATTTATTTATTCTGCGAAGTGATCTTCCGTCACAGGTATTTATTCGCGATA |
| | AGCTCATGGAGCGGCGTAACCGTCGCACAGGAAGGACAGAGAAAGCGCGGATCTG |
| | GGAAGTGACGGACAGAACGGTCAGGACCTGGATTGGGGAGGCGGTTGCCGCCGCT |
| | GCTGCTGACGGTGTGACGTTCTCTGTTCCGGTCACACCACATACGTTCCGCCATTCC |
| | TATGCGATGCACATGCTGTATGCCGGTATACCGCTGAAAGTTCTGCAAAGCCTGAT |
| | GGGACATAAGTCCATCAGTTCAACGGAAGTCTACACGAAGGTTTTTGCGCTGGATG |
| | TGGCTGCCCGGCACCGGGTGCAGTTTGCGATGCCGGAGTCTGATGCGGTTGCGATG |
| | CTGAAACAATTATCCTGAGAATAAATGCCTTGGCCTTTATATGGAAATGTGGAACT |
| | GAGTGGATATGCTGTTTTTGTCTGTTAAACAGAGAAGCTGGCTGTTATCCACTGAG |
| | AAGCGAACGAAACAGTCGGGAAAATCTCCCATTATCGTAGAGATCCGCATTATTAA |
| | TCTCAGGAGCCTGTGTAGCGTTTATAGGAAGTAGTGTTCTGTCATGATGCCTGCAA |
| | GCGGTAACGAAAACGATTTGAATATGCCTTCAGGAACAATAGAAATCTTCGTGCGG |
| | TGTTACGTTGAAGTGGAGCGGATTATGTCAGCAATGGACAGAACAACCTAATGAA |
| | CACAGAACCATGATGTGGTCTGTCCTTTTACAGCCAGTAGTGCTCGCCGCAGTCGA |
| | GCGACAGGGCGAAGCCCTCGGCTGGTTGCCCTCGCCGCTGGGCTGGCGGCCGTCTA |
| | TGGCCCTGCAAACGCGCCAGAAACGCCGTCGAAGCCGTGTGCGAGACACCGCGGC |
| | CGGCCGCCGGCGTTGTGGATACCTCGCGGAAAACTTGGCCCTCACTGACAGATGAG |
| | GGGCGGACGTTGACACTTGAGGGGCCGACTCACCCGGCGCGGCGTTGACAGATGA |
| | GGGGCAGGCTCGATTTCGGCCGGCGACGTGGAGCTGGCCAGCCTCGCAAATCGGC |
| | GAAAACGCCTGATTTTACGCGAGTTTCCCACAGATGATGTGGACAAGCCTGGGGAT |
| | AAGTGCCCTGCGGTATTGACACTTGAGGGGCGCGACTACTGACAGATGAGGGGCG |
| | CGATCCTTGACACTTGAGGGGCAGAGTGCTGACAGATGAGGGGCGCACCTATTGA |
| | CATTTGAGGGGCTGTCCACAGGCAGAAAATCCAGCATTTGCAAGGGTTTCCGCCCG |
| | TTTTTCGGCCACCGCTAACCTGTCTTTTAACCTGCTTTTAAACCAATATTTATAAAC |
| | CTTGTTTTTAACCAGGGCTGCGCCCTGTGCGCGTGACCGCGCACGCCGAAGGGGGG |
| | TGCCCCCCCTTCTCGAACCCTCCCGGTCGAGTGAGCGAGGAAGCACCAGGGAACA |
| | GCACTTATATATTCTGCTTACACACGATGCCTGAAAAAACTTCCCTTGGGGTTATCC |
| | ACTTATCCACGGGGATATTTTTATAATTATTTTTTTTATAGTTTTTAGATCTTCTTTTT |
| | TAGAGCGCCTTGTAGGCCTTTATCCATGCTGGTTCTAGAGAAGGTGTTGTGACAAA |
| | TTGCCCTTTCAGTGTGACAAATCACCCTCAAATGACAGTCCTGTCTGTGACAAATTG |
| | CCCTTAACCCTGTGACAAATTGCCCTCAGAAGAAGCTGTTTTTTCACAAAGTTATCC |
| | CTGCTTATTGACTCTTTTTTATTTAGTGTGACAATCTAAAAACTTGTCACACTTCAC |
| | ATGGATCTGTCATGGCGGAAACAGCGGTTATCAATCACAAGAAACGTAAAAATAG |
| | CCCGCGAATCGTCCAGTCAAACGACCTCACTGAGGCGGCATATAGTCTCTCCCGGG |
| | ATCAAAAACGTATGCTGTATCTGTTCGTTGACCAGATCAGAAAATCTGATGGCACC |
| | CTACAGGAACATGACGGTATCTGCGAGATCCATGTTGCTAAATATGCTGAAATATT |
| | CGGATTGACCTCTGCGGAAGCCAGTAAGGATATACGGCAGGCATTGAAGAGTTTC |
| | GCGGGGAAGGAAGTGGTTTTTTATCGCCCTGAAGAGGATGCCGGCGATGAAAAAG |
| | GCTATGAATCTTTTCCTTGGTTTATCAAACGTGCGCACAGTCCATCCAGAGGGCTTT |
| | ACAGTGTACATATCAACCCATATCTCATTCCCTTCTTTATCGGGTTACAGAACCGGT |
| | TTACGCAGTTTCGGCTTAGTGAAACAAAAGAAATCACCAATCCGTATGCCATGCGT |
| | TTATACGAATCCCTGTGTCAGTATCGTAAGCCGGATGGCTCAGGCATCGTCTCTCTG |
| | AAAATCGACTGGATCATAGAGCGTTACCAGCTGCCTCAAAGTTACCAGCGTATGCC |
| | TGACTTCCGCCGCCGCTTCCTGCAGGTCTGTGTTAATGAGATCAACAGCAGAACTC |
| | CAATGCGCCTCTCATACATTGAGAAAAAGAAAGGCCGCCAGACGACTCATATCGT |
| | ATTTTCCTTCCGCGATATCACTTCCATGACGACAGGATAGTCTGAGGGTTATCTGTC |
| | ACAGATTTGAGGGTGGTTCGTCACATTTGTTCTGACCTACTGAGGGTAATTTGTCAC |
| | AGTTTTGCTGTTTCCTTCAGCCTGCATGGATTTTCTCATACTTTTTGAACTGTAATTT |
| | TTAAGGAAGCCAAATTTGAGGGCAGTTTGTCACAGTTGATTTCCTTCTCTTTCCCTT |
| | CGTCATGTGACCTGATATCGGGGGTTAGTTCGTCATCATTGATGAGGGTTGATTATC |
| | ACAGTTTATTACTCTGAATTGGCTATCCGCGTGTGTACCTCTACCTGGAGTTTTTCC |
| | CACGGTGGATATTTCTTCTTGCGCTGAGCGTAAGAGCTATCTGACAGAACAGTTCT |
| | TCTTTGCTTCCTCGCCAGTTCGCTCGCTATGCTCGGTTACACGGCTGCGGCGAGCGC |
| | TAGTGATAATAAGTGACTGAGGTATGTGCTCTTCTTATCTCCTTTTGTAGTGTTGCT |
| | CTTATTTTAAACAACTTTGCGGTTTTTTGATGACTTTGCGATTTTGTTGTTGCTTTGC |
| | AGTAAATTGCAAGATTTAATAAAAAAACGCAAAGCAATGATTAAAGGATGTTCAG |
| | AATGAAACTCATGGAAACACTTAACCAGTGCATAAACGCTGGTCATGAAATGACG |
| | AAGGCTATCGCCATTGCACAGTTTAATGATGACAGCCCGGAAGCGAGGAAAATAA |
| | CCCGGCGCTGGAGAATAGGTGAAGCAGCGGATTTAGTTGGGGTTTCTTCTCAGGCT |
| | ATCAGAGATGCCGAGAAAGCAGGGCGACTACCGCACCCGGATATGGAAATTCGAG |
| | GACGGGTTGAGCAACGTGTTGGTTATACAATTGAACAAATTAATCATATGCGTGAT |
| | GTGTTTGGTACGCGATTGCGACGTGCTGAAGACGTATTTCCACCGGTGATCGGGGT |
| | TGCTGCCCATAAAGGTGGCGTTTACAAAACCTCAGTTTCTGTTCATCTTGCTCAGGA |
| | TCTGGCTCTGAAGGGGCTACGTGTTTTGCTCGTGGAAGGTAACGACCCCCAGGGAA |
| | CAGCCTCAATGTATCACGGATGGGTACCAGATCTTCATATTCATGCAGAAGACACT |
| | CTCCTGCCTTTCTATCTTGGGGAAAAGGACGATGTCACTTATGCAATAAAGCCCAC |
| | TTGCTGGCCGGGGCTTGACATTATTCCTTCCTGTCTGGCTCTGCACCGTATTGAAAC |
| | TGAGTTAATGGGCAAATTTGATGAAGGTAAACTGCCCACCGATCCACACCTGATGC |
| | TCCGACTGGCCATTGAAACTGTTGCTCATGACTATGATGTCATAGTTATTGACAGC |
| | GCGCCTAACCTGGGTATCGGCACGATTAATGTCGTATGTGCTGCTGATGTGCTGAT |
| | TGTTCCCACGCCTGCTGAGTTGTTTGACTACACCTCCGCACTGCAGTTTTTCGATAT |
| | GCTTCGTGATCTGCTCAAGAACGTTGATCTTAAAGGGTTCGAGCCTGATGTACGTA |
| | TTTTGCTTACCAAATACAGCAATAGTAATGGCTCTCAGTCCCCGTGGATGGAGGAG |
| | CAAATTCGGGATGCCTGGGGAAGCATGGTTCTAAAAAATGTTGTACGTGAAACGG |
| | ATGAAGTTGGTAAAGGTCAGATCCGGATGAGAACTGTTTTTGAACAGGCCATTGAT |
| | CAACGCTCTTCAACTGGTGCCTGGAGAAATGCTCTTTCTATTTGGGAACCTGTCTGC |
| | AATGAAATTTTCGATCGTCTGATTAAACCACGCTGGGAGATTAGATAATGAAGCGT |
| | GCGCCTGTTATTCCAAAACATACGCTCAATACTCAACCGGTTGAAGATACTTCGTT |
| | ATCGACACCAGCTGCCCCGATGGTGGATTCGTTAATTGCGCGCGTAGGAGTAATGG |
| | CTCGCGGTAATGCCATTACTTTGCCTGTATGTGGTCGGGATGTGAAGTTTACTCTTG |
| | AAGTGCTCCGGGGTGATAGTGTTGAGAAGACCTCTCGGGTATGGTCAGGTAATGAA |
| | CGTGACCAGGAGCTGCTTACTGAGGACGCACTGGATGATCTCATCCCTTCTTTTCTA |
| | CTGACTGGTCAACAGACACCGGCGTTCGGTCGAAGAGTATCTGGTGTCATAGAAAT |
| | TGCCGATGGGAGTCGCCGTCGTAAAGCTGCTGCACTTACCGAAAGTGATTATCGTG |
| | TTCTGGTTGGCGAGCTGGATGATGAGCAGATGGCTGCATTATCCAGATTGGGTAAC |
| | GATTATCGCCCAACAAGTGCTTATGAACGTGGTCAGCGTTATGCAAGCCGATTGCA |
| | GAATGAATTTGCTGGAAATATTTCTGCGCTGGCTGATGCGGAAAATATTTCACGTA |
| | AGATTATTACCCGCTGTATCAACACCGCCAAATTGCCTAAATCAGTTGTTGCTCTTT |
| | TTTCTCACCCCGGTGAACTATCTGCCCGGTCAGGTGATGCACTTCAAAAAGCCTTTA |
| | CAGATAAAGAGGAATTACTTAAGCAGCAGGCATCTAACCTTCATGAGCAGAAAAA |
| | AGCTGGGGTGATATTTGAAGCTGAAGAAGTTATCACTCTTTTAACTTCTGTGCTTAA |
| | AACGTCATCTGCATCAAGAACTAGTTTAAGCTCACGACATCAGTTTGCTCCTGGAG |
| | CGACAGTATTGTATAAGGGCGATAAAATGGTGCTTAACCTGGACAGGTCTCGTGTT |
| | CCAACTGAGTGTATAGAGAAAATTGAGGCCATTCTTAAGGAACTTGAAAAGCCAG |
| | CACCCTGATGCGACCACGTTTTAGTCTACGTTTATGTGTCTTTACTTAATGTCCTTTG |
| | TTACAGGCCAGAAAGCATAACTGGCCTGAATATTCTCTCTGGGCCCACTGTTCCAC |
| | TTGTATCGTCGGTCTGATAATCAGACTGGGACCACGGTCCCACTCGTATCGTCGGT |
| | CTGATTATTAGTCTGGGACCACGGTCCCACTCGTATCGTCGGTCTGATTATTAGTCT |
| | GGGACCACGGTCCCACTCGTATCGTCGGTCTGATAATCAGACTGGGACCACGGTCC |
| | CACTCGTATCGTCGGTCTGATTATTAGTCTGGGACCATGGTCCCACTCGTATCGTCG |
| | GTCTGATTATTAGTCTGGGACCACGGTCCCACTCGTATCGTCGGTCTGATTATTAGT |
| | CTGGAACCACGGTCCCACTCGTATCGTCGGTCTGATTATTAGTCTGGGACCACGGT |
| | CCCACTCGTATCGTCGGTCTGATTATTAGTCTGGGACCACGATCCCACTCGTGTTGT |
| | CGGTCTGATTATCGGTCTGGGACCACGGTCCCACTTGTATTGTCGATCAGACTATCA |
| | GCGTGAGACTACGATTCCATCAATGCCTGTCAAGGGCAAGTATTGACATGTCGTCG |
| | TAACCTGTAGAACGGAGTAACCTCGGTGTGCGGTTGTATGCCTGCTGTGGATTGCT |
| | GCTGTGTCCTGCTTATCCACAACATTTTGCGCACGGTTATGTGGACAAAATACCTG |
| | GTTACCCAGGCCGTGCCGGCACGTTAACCGGGCTGCATCCGATGCAAGTGTGTCGC |
| | TGTCGACGAGCTCGCGAGCTCGGACATGAGGTTGCCCCGTATTCAGTGTCGCTGAT |
| | TTGTATTGTCTGAAGTTGTTTTTACGTTAAGTTGATGCAGATCAATTAATACGATAC |
| | CTGCGTCATAATTGATTATTTGACGTGGTTTGATGGCCTCCACGCACGTTGTGATAT |
| | GTAGATGATAATCATTATCACTTTACGGGTCCTTTCCGGTGATCCGACAGGTTACG |
| | GGGCGGCGACCTCGCGGGTTTTCGCTATTTATGAAAATTTTCCGGTTTAAGGCGTTT |
| | CCGTTCTTCTTCGTCATAACTTAATGTTTTTATTTAAAATACCCTCTGAAAAGAAAG |
| | GAAACGACAGGTGCTGAAAGCGAGCTTTTTGGCCTCTGTCGTTTCCTTTCTCTGTTT |
| | TTGTCCGTGGAATGAACAATGGAAGTCCGAGCTCATCGCTAATAACTTCGTATAGC |
| | ATACATTATACGAAGTTATATTCGAT |
| SEQ ID NO: | CGAGCGCCATCTCGAACCGACGTTGCTGGCCGTACATTTGTACGGCTCCGCAGTGG |
| 7. ABCA4 | ATGGCGGCCTGAAGCCACACAGTGATATTGATTTGCTGGTTACGGTGACCGTAAGG |
| plasmid | CTTGATGAAACAACGCGGCGAGCTTTGATCAACGACCTTTTGGAAACTTCGGCTTC |
| template | CCCTGGAGAGAGCGAGATTCTCCGCGCTGTAGAAGTCACCATTGTTGTGCACGACG |
| | ACATCATTCCGTGGCGTTATCCAGCTAAGCGCGAACTGCAATTTGGAGAATGGCAG |
| | CGCAATGACATTCTTGCAGGTATCTTCGAGCCAGCCACGATCGACATTGATCTGGC |
| | TATCTTGCTGACAAAAGCAAGAGAACATAGCGTTGCCTTGGTAGGTCCAGCGGCGG |
| | AGGAACTCTTTGATCCGGTTCCTGAACAGGATCTATTTGAGGCGCTAAATGAAACC |
| | TTAACGCTATGGAACTCGCCGCCCGACTGGGCTGGCGATGAGCGAAATGTAGTGCT |
| | TACGTTGTCCCGCATTTGGTACAGCGCAGTAACCGGCAAAATCGCGCCGAAGGATG |
| | TCGCTGCCGACTGGGCAATGGAGCGCCTGCCGGCCCAGTATCAGCCCGTCATACTT |
| | GAAGCTAGACAGGCTTATCTTGGACAAGAAGAAGATCGCTTGGCCTCGCGCGCAG |
| | ATCAGTTGGAAGAATTTGTCCACTACGTGAAAGGCGAGATCACCAAGGTAGTCGG |
| | CAAATAACCaacGATCTGTTGATCAGCAGTTCAACCTGTTGATAGTACGTACTAAGC |
| | TCTCATGTTTCACGTACTAAGCTCTCATGTTTAACGTACTAAGCTCTCATGTTTAAC |
| | GAACTAAACCCTCATGGCTAACGTACTAAGCTCTCATGGCTAACGTACTAAGCTCT |
| | CATGTTTCACGTACTAAGCTCTCATGTTTGAACAATAAAATTAATATAAATCAGCA |
| | ACTTAAATAGCCTCTAAGGTTTTAAGTTTTATAAGAAAAAAAAGAATATATAAGGC |
| | TTTTAAAGCTTTTAAGGTTTAACGGTTGTGGACAACAAGCCAGGGATGTAACGCAC |
| | TGAGAAGCCCTTAGAGCCTCTCAAAGCAATTTTGAGTGACACAGGAACACTTAACG |
| | GCTGACATGGgaattagccatgggcccgtgcgaatcactatatCagaccgctgatccttcaactcagcaaaagtt |
| | cgatttattcaacaaagccacgttgtgtctcaaaatctctgatgttacattgcacaagataaaaatatatcatca |
| | tgaacaatanaactgtctgcttacataaacagtaatacaaggggtgttatgagccatattcaacgggaaacgtcg |
| | aggccgcgattaaattccaacatggatgctgatttatatgggtataaatgggctcgcgataatgtcgggcaatca |
| | ggtgcgacaatctatcgcttgtatgggaagcccgatgcgccagagttgtttctgaaacatggcaaaggtagcgtt |
| | gccaatgatgttacagatgagatggtcagactaaactggctgacggaatttatgcctcttccgaccatcaagcat |
| | tttatccgtactcctgatgatgcatggttactcaccactgcgatccccggaaaaacagcattccaggtattagaa |
| | gaatatcctgattcaggtgaaaatattgttgatgcgctggcagtgttcctgcgccggttgcattcgattcctgtt |
| | tgtaattgtccttttaacagcgatcgcgtatttcgtctcgctcaggcgcaatcacgaatgaataacggtttggtt |
| | gatgcgagtgattttgatgacgagcgtaatggctggcctgttgaacaagtctggaaagaaatgcataaacttttg |
| | ccattctcaccggattcagtcgtcactcatggtgatttctcacttgataaccttatttttgacgaggggaaatta |
| | ataggttgtattgatgttggacgagtcggaatcgcagaccgataccaggatcttgccatcctatggaactgcctc |
| | ggtgagttttctccttcattacagaaacggctttttcaaaaatatggtattgataatcctgatatgaataaattg |
| | cagtttcatttgatgctcgatgagtttttctaatcagaattggttaattggttgtaacactggcagagcattacg |
| | ctgacttgacgggagtatcacgaggcagaatttcagataaaaaaaatccttagctttcgctaaggatgatttctg |
| | gaattcgcggccgcttctagagtccctatcagtgatagagattgacatccctatcagtgatagagatactgagca |
| | ctactagagaaagaggagaaatactagatggcttcctccgaagacgttatcaaagagttcatgcgtttcaaagtt |
| | cgtatggaaggttccgttaacggtcacgagttcgaaatcgaaggtgaaggtgaaggtcgtccgtacgaaggtacc |
| | cagaccgctaaactgaaagttaccaaaggtggtccgctgccgttcgcttgggacatcctgtccccgcagttccag |
| | tacggttccaaagcttacgttaaacacccggctgacatcccggactacctgaaactgtccttcccggaaggtttc |
| | aaatgggaacgtgttatgaacttcgaagacggtggtgttgttaccgttacccaggactcctccctgcaagacggt |
| | gagttcatctacaaagttaaactgcgtggtaccaacttcccgtccgacggtccggttatgcagaaaaaaaccatg |
| | ggttgggaagcttccaccgaacgtatgtacccggaagacggtgctctgaaaggtgaaatcasaatgcgtctgaaa |
| | ctgaaagacggtggtcactacgacgctgaagttaaaaccacctacatggctaaaaaaccggttcagctgccgggt |
| | gcttacaaaaccgacatcaaactggacatcacctcccacaacgaagactacaccatcgttgaacagtacgaacgt |
| | gctgaaggtcgtcactccaccggtgcttaataacgctgatagtgctagtgtagatcgctactagagccaggcatc |
| | aaataaaacgaaaggctcagtcgaaagactgggcctttcgttttatctgttgtttgtcggtgaacgctctctact |
| | agagtcacactggctcaccttcggggggcctttctgcgtttatatactagtaggatcctaactcgacggcaggct |
| | tcctcgctcactgactcgctgcgctcggtcgttcggctgcggcgagcggtatcagctcactcaaaggcggtaatg |
| | gcttgtcgacgacggcggactccgtcgtcaggatcataggggctgttatctgataaggcttatctggtctcattt |
| | tCTCTTCGgtatcacaggagaatttcagGGAGACATTGATTATTGA |
| | CTAGTTATTAATAGTAATCAATTACGGGGTCATTAGTTCATAGCCCATATATGGAG |
| | TTCCGCGTTACATAACTTACGGTAAATGGCCCGCCTGGCTGACCGCCCAACGACCC |
| | CCGCCCATTGACGTCAATAATGACGTATGTTCCCATAGTAACGCCAATAGGGACTT |
| | TCCATTGACGTCAATGGGTGGAGTATTTACGGTAAACTGCCCACTTGGCAGTACAT |
| | CAAGTGTATCATATGCCAAGTACGCCCCCTATTGACGTCAATGACGGTAAATGGCC |
| | CGCCTGGCATTATGCCCAGTACATGACCTTATGGGACTTTCCTACTTGGCAGTACAT |
| | CTACGTATTAGTCATCGCTATTACCATGTCGAGGTGAGCCCCACGTTCTGCTTCACT |
| | CTCCCCATCTCCCCCCCCTCCCCACCCCCAATTTTGTATTTATTTATTTTTTAATTAT |
| | TTTGTGCAGCGATGGGGGGGGGGGGGGGGGGGGGGGGCGCGCCAGGCGGGGCGG |
| | GGCGGGGCGAGGGGGGGGGGGGGGCGAGGCGGAGAGGTGCGGCGGCAGCCAATC |
| | AGAGCGGCGCGCTCCGAAAGTTTCCTTTTATGGCGAGGCGGCGGCGGCGGCGGCC |
| | CTATAAAAAGCGAAGCGCGCGGCGGGCGGGAGTCGCTGCGACGCTGCCTTCGCCC |
| | CGTGCCCCGCTCCGCCGCCGCCTCGCGCCGCCCGCCCCGGCTCTGACTGACCGCGT |
| | TACTCCCACAGGTGAGCGGGCGGGACGGCCCTTCTCCTCCGGGCTGTAATTAGCGC |
| | TTGGTTTAATGACGGCTTGTTTCTTTTCTGTGGCTGCGTGAAAGCCTTGAGGGGCTC |
| | CGGGAGGGCCCTTTGTGCGGGGGGAGCGGCTCGGGGGGTGCGTGCGTGTGTGTGT |
| | GCGTGGGGAGCGCCGCGTGCGGCTCCGCGCTGCCCGGCGGCTGTGAGCGCTGCGG |
| | GCGCGGCGCGGGGCTTTGTGCGCTCCGCAGTGTGCGCGAGGGGAGCGCGGCCGGG |
| | GGCGGTGCCCCGCGGTGCGGGGGGGGCTGCGAGGGGAACAAAGGCTGCGTGCGGG |
| | GTGTGTGCGTGGGGGGGTGAGCAGGGGGTGTGGGCGCGTCGGTCGGGCTGCAACC |
| | CCCCCTGCACCCCCCTCCCCGAGTTGCTGAGCACGGCCCGGCTTCGGGTGCGGGGC |
| | TCCGTACGGGGCGTGGCGCGGGGCTCGCCGTGCCGGGGGGGGGGGGCGGCAGGT |
| | GGGGGTGCCGGGCGGGGGGGGCCGCCTCGGGCCGGGGAGGGCTCGGGGGAGGG |
| | GCGCGGCGGCCCCCGGAGCGCCGGCGGCTGTCGAGGCGCGGCGAGCCGCAGCCAT |
| | TGCCTTTTATGGTAATCGTGCGAGAGGGCGCAGGGACTTCCTTTGTCCCAAATCTGT |
| | GCGGAGCCGAAATCTGGGAGGCGCCGCCGCACCCCCTCTAGCGGGCGCGGGGCGA |
| | AGCGGTGCGGCGCCGGCAGGAAGGAAATGGGGGGGGAGGGCCTTCGTGCGTCGCC |
| | GCGCCGCCGTCCCCTTCTCCCTCTCCAGCCTCGGGGCTGTCCGCGGGGGGACGGCT |
| | GCCTTCGGGGGGGACGGGGCAGGGCGGGGTTCGGCTTCTGGCGTGTGACCGGCGG |
| | CTCTAGACAATTGTACTAACCTTCTTCTCTTTCCTCTCCTGACAGGGAGTTTAAACA |
| | GATAAGTTTGTACAAAAAAGAGAGGTGCCACCATGGGCTTTGTGCGACAGATTCA |
| | GCTGCTGCTGTGGAAGAACTGGACCCTGCGGAAGCGGCAGAAAATCAGATTCGTG |
| | GTGGAACTCGTGTGGCCCCTGAGCCTGTTTCTGGTGCTGATCTGGCTGCGGAACGC |
| | CAATCCTCTGTACAGCCACCACGAGTGTCACTTCCCCAACAAGGCCATGCCTTCTG |
| | CCGGAATGCTGCCTTGGCTGCAGGGCATCTTCTGCAACGTGAACAACCCCTGCTTT |
| | CAGAGCCCCACACCTGGCGAAAGCCCTGGCATCGTGTCCAACTACAACAACAGCA |
| | TCCTGGCCAGAGTGTACCGGGACTTCCAAGAGCTGCTGATGAACGCCCCTGAGTCT |
| | CAGCACCTGGGCAGAATCTGGACCGAGCTGCACATCCTGAGCCAGTTCATGGACAC |
| | CCTGAGAACACACCCCGAGAGAATCGCCGGCAGGGGCATCAGAATCCGGGACATC |
| | CTGAAGGACGAGGAAACCCTGACACTGTTCCTCATCAAGAACATCGGCCTGAGCG |
| | ACAGCGTGGTGTACCTGCTGATCAACAGCCAAGTGCGGCCCGAGCAGTTTGCTCAT |
| | GGCGTGCCgGATCTCGCCCTGAAGGATATCGCCTGTTCTGAGGCCCTGCTGGAACG |
| | GTTCATCATCTTCAGCCAGCGGAGAGGCGCCAAGACCGTCAGATATGCCCTGTGCA |
| | GTCTGAGCCAGGGAACCCTGCAGTGGATCGAGGATACCCTGTACGCCAACGTGGA |
| | CTTCTTCAAGCTGTTCCGGGTGCTGCCCACACTGCTGGATTCTAGATCCCAGGGCAT |
| | CAACCTGAGAAGCTGGGGCGGCATCCTGTCCGACATGAGCCCAAGAATCCAAGAG |
| | TTCATCCACCGGCCTAGCATGCAGGACCTGCTGTGGGTTACCAGACCTCTGATGCA |
| | GAACGGCGGACCCGAGACATTCACCAAGCTGATGGGCATTCTGAGCGATCTGCTGT |
| | GCGGCTACCCTGAAGGCGGAGGATCTAGAGTGCTGAGCTTCAATTGGTACGAGGA |
| | CAACAACTACAAGGCCTTCCTGGGCATCGACTCCACCAGAAAGGACCCCATCTACA |
| | GCTACGACCGGCGGACAACCAGCTTCTGCAATGCCCTGATCCAGAGCCTGGAAAG |
| | CAACCCTCTGACCAAGATCGCTTGGAGGGCCGCCAAACCTCTGCTGATGGGAAAG |
| | ATCCTGTACACCCCTGACAGCCCTGCCGCCAGAAGAATCCTGAAGAACGCCAACA |
| | GCACCTTCGAGGAACTGGAACACGTGCGCAAGCTGGTCAAGGCCTGGGAAGAAGT |
| | GGGACCTCAGATITGGTACTTCTTCGACAATAGCACCCAGATGAACATGATCAGAG |
| | ACACCCTGGGCAACCCTACCGTGAAGGACTTCCTGAACAGACAGCTGGGCGAAGA |
| | GGGCATTACCGCCGAGGCCATCCTGAACTTTCTGTACAAGGGCCCCAGAGAGTCCC |
| | AGGCCGACGACATGGCCAACTTCGATTGGGGGACATCTTCAACATCACCGACAG |
| | AACCCTGCGGCTGGTCAACCAGTACCTGGAATGCCTGGTGCTGGACAAGTTCGAGA |
| | GCTACAACGACGAGACACAGCTGACCCAGAGAGCCCTGTCTCTGCTGGAAGAGAA |
| | TATGTTCTGGGCTGGCGTGGTGTTCCCCGACATGTACCCTTGGACAAGCAGCCTGC |
| | CTCCTCACGTGAAGTACAAGATCCGGATGGACATCGACGTGGTCGAAAAGACCAA |
| | CAAGATCAAGGACCGGTACTGGGACAGCGGCCCTAGAGCTGATCCCGTGGAAGAT |
| | TTTCGGTACATCTGGGGCGGATTCGCATACCTGCAGGACATGGTGGAACAGGGAAT |
| | CACACGGTCCCAGGTGCAGGCTGAAGCTCCTGTGGGAATCTACCTGCAGCAGATGC |
| | CTTATCCTTGCTTCGTGGACGACAGCTTCATGATCATCCTGAATCGGTGCTTCCCCA |
| | TCTTCATGGTGCTGGCCTGGATCTACTCCGTGTCTATGACCGTGAAGTCCATCGTGC |
| | TGGAAAAAGAGCTGCGGCTGAAAGAGACACTGAAGAACCAGGGCGTGTCCAATGC |
| | CGTGATCTGGTGCACCTGGTTTCTGGACAGCTTCTCCATTATGAGCATGAGCATCTT |
| | TCTGCTGACGATCTTCATCATGCACGGCCGaATCCTGCACTACAGCGACCCCTTTAT |
| | CCTCTTCCTGTTCCTGCTGGCCTTCAGCACCGCTACAATCATGCTGTGTTTTCTGCTG |
| | TCCACCTTCTTCAGCAAGGCCTCTCTGGCCGCTGCTTGTAGCGGCGTGATCTACTTC |
| | ACCCTGTACCTGCCTCACATCCTGTGCTTCGCATGGCAGGACAGAATGACCGCCGA |
| | GCTGAAGAAAGCTGTGTCCCTGCTGAGCCCTGTGGCCTTTGGCTTTGGCACCGAGT |
| | ACCTCGTCAGATTTGAGGAACAAGGACTGGGACTGCAGTGGTCCAACATCGGCAA |
| | TAGCCCTACAGAGGGCGACGAGTTCAGCTTCCTGCTGTCTATGCAGATGATGCTGC |
| | TGGACGCCGCCGTGTATGGACTGCTGGCTTGGTATCTGGACCAGGTGTTCCCAGGC |
| | GATTACGGCACTCCTCTGCCTTGGTATTTCCTGCTGCAAGAGAGCTACTGGCTCGGC |
| | GGCGAGGGATGTAGCACCAGAGAAGAAAGAGCCCTGGAAAAGACCGAGCCTCTG |
| | ACCGAGGAAACAGAGGACCCTGAACACCCAGAGGGCATCCACGATAGCTTTTTCG |
| | AGAGAGAACACCCCGGCTGGGTGCCAGGCGTGTGTGTGAAGAATCTGGTCAAGATt |
| | TTCGAGCCCTGCGGCAGACCTGCCGTGGACAGACTGAACATCACCTTCTACGAGAA |
| | CCAGATTACCGCCTTTCTGGGCCACAACGGCGCTGGCAAGACAACCACAtTGAGCA |
| | TCCTCACAGGCCTGCTGCCTCCAACAAGCGGCACAGTTCTCGTTGGCGGCAGAGAC |
| | ATCGAGACAAGCCTGGATGCCGTCAGACAGTCCCTGGGCATGTGCCCTCAGCACAA |
| | CATCCTGTTTCACCACCTGACCGTGGCCGAGCACATGCTGTTTTATGCCCAGCTGAA |
| | GGGCAAGAGCCAAGAAGAGGCTCAGCTGGAAATGGAAGCCATGtTgGAGGACACC |
| | GGCCTGCACCACAAGAGAAATGAGGAAGCCCAGGATCTGAGCGGCGGCATGCAGA |
| | GAAAACTGAGCGTGGCCATTGCCTTCGTGGGCGACGCCAAGGTTGTGATCCTGGAT |
| | GAGCCTACAAGCGGCGTGGACCCTTACAGCAGAAGATCCATCTGGGATCTGCTGCT |
| | GAAGTACAGAtcaGGCCGGACCATCATCATGAGCACCCACCACATGGACGAGGCCG |
| | ATCTGCTCGGAGACAGAATCGCCATCATTGCTCAGGGCAGACTGTACTGCAGCGGC |
| | ACCCCACTGTTTCTGAAGAACTGTTTCGGCACCGGACTGTATCTGACCCTCGTGCG |
| | GAAGATGAAGAACATCCAGTCTCAGCGGAAGGGCAGCGAGGGCACCTGTAGCTGT |
| | TCTAGCAAGGGCTTTAGCACCACCTGTCCAGCTCACGTGGACGATCTGACCCCTGA |
| | ACAGGTGCTGGATGGCGACGTGAACGAGCTGATGGACGTGGTGCTGCACCATGTG |
| | CCTGAGGCCAAGCTGGTGGAATGCATCGGCCAAGAACTGATTTTTCTGCTCCCGAA |
| | CAAGAACTTCAAGCACCGGGCCTACGCCAGCCTGTTCAGAGAGCTGGAAGAAACC |
| | CTGGCCGACCTGGGCCTGTCTAGCTTTGGCATCAGCGACACCCCTCTCGAAGAGATt |
| | TTCCTGAAAGTGACAGAGGACAGCGATAGCGGCCCTCTGTTTGCTGGCGGAGCACA |
| | GCAAAAGCGCGAGAACGTGAACCCTAGACACCCCTGTCTGGGCCCAAGAGAGAAA |
| | GCCGGACAGACCCCTCAGGACAGCAATGTGTGCTCTCCTGGTGCTCCTGCCGCTCA |
| | TCCTGAGGGACAACCTCCACCTGAACCTGAGTGTCCTGGACCTCAGCTGAACACCG |
| | GAACACAGCTGGTTCTGCAGCACGTGCAGGCTCTGCTCGTGAAGAGATTCCAGCAC |
| | ACCATCAGAAGCCACAAGGACTTTCTGGCCCAGATCGTGCTGCCCGCCACCTTTGT |
| | TTTTCTGGCTCTGATGCTGAGCATCGTGATCCCTCCATTCGGCGAGTACCCCGCTCT |
| | GACACTGCACCCTTGGATCTACGGCCAGCAGTACACCTTTTTCTCCATGGACGAAC |
| | CCGGCAGCGAGCAGTTCACAGTGCTGGCTGATGTCCTGCTGAACAAGCCCGGCTTC |
| | GGCAACCGGTGTCTGAAAGAAGGATGGCTGCCTGAGTACCCTTGCGGCAACAGCA |
| | CACCTTGGAAAACCCCTAGCGTGTCCCCTAACATCACCCAGCTGTTCCAAAAGCAG |
| | AAATGGACCCAAGTGAACCCCTCTCCATCCTGCCGGTGCTCCACAAGGGAAAAGCT |
| | GACCATGCTGCCCGAGTGTCCAGAAGGCGCTGGCGGACTTCCTCCACCTCAGAGAA |
| | CACAGAGATCCACCGAGATTCTCCAGGACCTGACCGACCGGAATATCAGCGACTTC |
| | CTGGTTAAGACATACCCCGCACTGATCCGGTCCAGCCTGAAGTCCAAGTTCTGGGT |
| | CAACGAACAGAGATACGGCGGCATCAGCATCGGCGGAAAACTGCCTGTGGTGCCT |
| | ATCACAGGCGAGGCCCTTGTGGGCTTTCTGTCCGATCTGGGGAGAATCATGAACGT |
| | GTCCGGCGGACCTATCACCAGGGAAGCCAGCAAAGAGATCCCCGATTTCCTGAAG |
| | CACCTGGAAACCGAGGACAATATCAAAGTGTGGTTCAACAACAAAGGATGGCACG |
| | CCCTCGTGTCTTTTCTGAACGTGGCCCACAATGCCATCCTGCGGGCTAGCCTGCCTA |
| | AGGACAGAAGCCCTGAGGAATACGGCATCACCGTGATCTCCCAGCCTCTGAATCTG |
| | ACCAAAGAGCAGCTGAGCGAGATCACCGTGCTGACCACCTCTGTGGATGCTGTGGT |
| | GGCCATCTGCGTGATCTTCAGCATGAGCTTCGTGCCCGCCTCCTTCGTGCTGTACCT |
| | GATTCAAGAGAGAGTGAACAAGAGCAAGCACCTCCAGTTCATCTCCGGGGTGTCC |
| | CCAACCACCTACTGGGTCACCAATTTTCTGTGGGACATCATGAACTACAGCGTGTC |
| | AGCCGGCCTGGTCGTGGGCATCTTTATCGGCTTTCAGAAGAAGGCCTACACGAGCC |
| | CCGAGAACCTGCCTGCTTTGGTTGCTCTGCTGCTCCTGTATGGCTGGGCCGTGATTC |
| | CCATGATGTACCCCGCCAGCTTTCTGTTTGACGTGCCCAGCACAGCCTACGTGGCC |
| | CTGTCTTGCGCCAATCTGTTCATCGGCATCAACAGCAGCGCCATCACATTCATCCTG |
| | GAACTGTTCGAGAACAACAGGACCCTGCTGCGGTTCAACGCCGTGCTGCGGAAACT |
| | GCTGATCGTGTTCCCTCACTTCTGTCTCGGCAGAGGCCTGATCGACCTGGCTCTGTC |
| | TCAGGCCGTGACCGATGTGTACGCCAGATTTGGCGAGGAACACTCCGCCAATCCAT |
| | TCCACTGGGACCTGATCGGCAAGAACCTGTTCGCCATGGTGGTGGAAGGCGTCGTG |
| | TACTTCCTGCTCACTCTGCTGGTGCAGAGACACTTTTTTCTGTCCCAATGGATCGCC |
| | GAGCCTACCAAAGAACCCATTGTGGACGAGGACGACGATGTGGCCGAGGAAAGAC |
| | AGAGAATCATCACCGGCGGCAACAAGACCGATATCCTGAGACTGCACGAGCTGAC |
| | AAAGATtTACCCCGGCACAAGCTCCCCAGCCGTGGATAGGCTTTGTGTGGGAGTTA |
| | GACCCGGCGAGTGCTTTGGCCTGCTGGGAGTTAATGGCGCCGGAAAGACCACCAC |
| | CTTCAAGATGCTGACCGGCGACACCACAGTGACAAGCGGAGATGCTACAGTGGCC |
| | GGCAAGAGCATCCTGACCAACATCAGCGAAGTGCATCAGAACATGGGCTACTGCC |
| | CTCAGTTCGACGCCATCGACGAACTGCTGACAGGCCGCGAACACCTGTATCTGTAT |
| | GCCAGACTGAGAGGCGTGCCCGCTGAAGAGATCGAGAAGGTGGCCAACTGGTCCA |
| | TCAAGTCTCTGGGCCTGACAGTGTACGCCGACTGTCTGGCCGGAACATACAGCGGA |
| | GGAAACAAGCGGAAGCTGAGCACCGCCATTGCTCTGATCGGATGCCCACCTCTGGT |
| | CCTGCTGGATGAACCCACCACCGGAATGGACCCCAGGCTAGAAGAATGCTCTGGA |
| | ACGTGATCGTGTCTATCATCCGCGAGGGCAGAGCTGTGGTGCTGACCTCTCACAGC |
| | ATGGAAGAGTGCGAGGCTCTGTGTACCCGGCTGGCCATTATGGTCAAGGGCGCCTT |
| | CAGATGCATGGGCACCATTCAGCATCTGAAAAGCAAGTTCGGCGACGGCTACATC |
| | GTGACAATGAAGATCAAGAGCCCCAAGGACGACCTCCTGCCTGATCTGAACCCCGT |
| | GGAACAGTTTTTTCAGGGCAACTTCCCCGGCTCCGTGCAGCGGGAAAGACACTATA |
| | ACATGCTGCAGTTTCAGGTGTCCTCCTCCAGCCTGGCTCGGATCTTTCAACTGCTGC |
| | TCTCTCACAAGGACAGCCTGCTGATTGAAGAGTACAGCGTGACACAGACCACACTC |
| | GACCAGGTTTTCGTGAACTTCGCCAAGCAGCAGACCGAGAGCCACGACCTGCCTCT |
| | GCATCCTAGAGCCGCTGGTGCCTCTAGACAAGCTCAGGACTAAGCTTCCACTGGAT |
| | TGTACAATTACATAAAATAAAATATCTTTATTTTCATTACATCTGTGTGTTGGTTTTT |
| | TGTGTGCGCTACTtttttgagaccggtttgtctggtcaaccaccgcggactcagtggtgtacggtacaaaccCCAT |
| | CCAGCTGATATCCttgacaGCTAGCtcagtCCTAGGtataatGCTAGCgGATCTCGTCAGTAACGAG |
| | CCCTGGGAGAAAACAAAATGAGCCATCTGGCAGAACTGGTTGCAAGCGCAAAAGC |
| | AGCAATTAGCCAGGCAAGTGATGTTGCAGCACTGGATAATGTTCGTGTTGAATATC |
| | TGGGTAAAAAGGGTCATCTGACCCTGCAGATGACCACACTGCGTGAACTGCCTCCG |
| | GAAGAACGTCCGGCAGCCGGTGCAGTTATTAATGAAGCAAAAGAACAGGTTCAGC |
| | AGGCACTGAATGCACGTAAAGCCGAACTGGAAAGCGCAGCCCTGAATGCCCGTCT |
| | GGCAGCAGAAACCATTGATGTTAGCCTGCCTGGTCGTCGTATTGAAAATGGTGGTC |
| | TGCATCCGGTTACACGTACCATTGATCGCATTGAAAGCTTTTTCGGTGAACTGGGTT |
| | TTACCGTTGCAACCGGTCCGGAAATTGAAGATGATTATCATAATTTCGATGCCCTG |
| | AACATTCCGGGTCATCATCCGGCACGTGCAGATCATGATACCTTTTGGTTTGATACC |
| | ACACGTCTGCTGCGTACCCAGACCAGCGGTGTTCAGATTCGTACCATGAAAGCACA |
| | GCAGCCTCCGATTCGTATTATTGCACCGGGTCGTGTTTATCGCAATGATTATGATCA |
| | GCTTCACCAATCTGAAAGGCACCCTGCATGATTTTCTGCGCAATTTCTTTGAAGAG |
| | GACCCATACGCCGATGTTTCATCAGATGGAAGGTCTGATTGTGGATACCAATATTA |
| | GCTTCACCAATCTGAAAGGCACCCTGCATGATTTTCTGCGCAATTTCTTTGAAGAG |
| | GACCTGCAGATTCGTTTTCGTCCGAGCTATTTTCCGTTTAGCGAACCGAGTGCCGAA |
| | GTTGATGTTATGGGCAAAAATGGTAAATGGCTGGAAGTTTTAGGTTGCGGTATGGT |
| | TCATCCGAATGTTCTGCGTAATGTTGGTATTGATCCGGAAGTTTATAGCGGTTTTGG |
| | TTTTGGTATGGGCATGGAACGTCTGACCATGCTGCGTTATGGTGTTACCGATCTGCG |
| | TAGCTTCTTTGAAAATGATCTGCGTTTTCTGAAACAGTTTAAATGAGGATCTccaggcatcaaataaaacga |
| | aaggctcagtcgaaagactgggcctttcgttttatctgttgtttgtcggtgaacgctctctactagagtcac |
| | actggctcaccttcgggtgggcctttctgcgtttataGCCTATAGTGAGTCGTATTACATGGTCATAGCTGTTT |
| | CCTGGCAGCTCTGGCCCGTGTCTCAAAATCTCTGATGTTACATTGCACAAGATAAAAATA |
| | TATCATCATGCCTCCTCTAGACCAGCCAGGACAGAAATGCCTCGACTTCGCTGCTG |
| | CCCAAGGTTGCCGGGTGACGCACACCGTGGAAACGGATGAAGGCACGAACCCAGT |
| | GGACATAAGCCTGTTCGGTTCGTAAGCTGTAATGCAAGTAGCGTATGCGCTCACGC |
| | AACTGGTCCAGAACCTTGACCGAACGCAGCGGTGGTAACGGCGCAGTGGCGGTTTT |
| | CATGGCTTGTTATGACTGTTTTTTTGGGGTACAGTCTATGCCTCGGGCATCCAAGCA |
| | GCAAGCGCGTTACGCCGTGGGTCGATGTTTGATGTTATGGAGCAGCAACGATGTTA |
| | CGCAGCAGGGCAGTCGCCCTAAAACAAAGTTAAACATCATGAGGGAAGCGGTGAT |
| | CGCCGAAGTATCGACTCAACTATCAGAGGTAGTTGGCGTCAT |
| SEQ ID NO: | CTCTTCGgtatcacaggagaatttcagGGAGACATTGATTATTGACTAGTTATTAATAGTAATC |
| 8. ABCA4 | AATTACGGGGTCATTAGTTCATAGCCCATATATGGAGTTCCGCGTTACATAACTTA |
| circular DNA | CGGTAAATGGCCCGCCTGGCTGACCGCCCAACGACCCCCGCCCATTGACGTCAATA |
| vector | ATGACGTATGTTCCCATAGTAACGCCAATAGGGACTTTCCATTGACGTCAATGGGT |
| | GGAGTATTTACGGTAAACTGCCCACTTGGCAGTACATCAAGTGTATCATATGCCAA |
| | GTACGCCCCCTATTGACGTCAATGACGGTAAATGGCCCGCCTGGCATTATGCCCAG |
| | TACATGACCTTATGGGACTTTCCTACTTGGCAGTACATCTACGTATTAGTCATCGCT |
| | ATTACCATGTCGAGGTGAGCCCCACGTTCTGCTTCACTCTCCCCATCTCCCCCCCCT |
| | CCCCACCCCCAATTTTGTATTTATTTATTTTTTAATTATTTTGTGCAGCGATGGGGGC |
| | GGGGGGGGGGGGGGGGCGCGCGCCAGGCGGGGCGGGGCGGGGGCGAGGGGCGGGG |
| | CGGGGCGAGGCGGAGAGGTGCGGCGGCAGCCAATCAGAGCGGCGCGCTCCGAAA |
| | GTTTCCTTTTATGGCGAGGCGGCGGCGGCGGCGGCCCTATAAAAAGCGAAGCGCG |
| | CGGCGGGCGGGAGTCGCTGCGACGCTGCCTTCGCCCCGTGCCCCGCTCCGCCGCCG |
| | CCTCGCGCCGCCCGCCCCGGCTCTGACTGACCGCGTTACTCCCACAGGTGAGCGGG |
| | CGGGACGGCCCTTCTCCTCCGGGCTGTAATTAGCGCTTGGTTTAATGACGGCTTGTT |
| | TCTTTTCTGTGGCTGCGTGAAAGCCTTGAGGGGCTCCGGGAGGGCCCTTTGTGCGG |
| | GGGGAGCGGCTCGGGGGGTGCGTGCGTGTGTGTGTGCGTGGGGAGCGCCGCGTGC |
| | GGCTCCGCGCTGCCCGGCGGCTGTGAGCGCTGCGGGCGCGGCGCGGGGCTTTGTGC |
| | GCTCCGCAGTGTGCGCGAGGGGAGCGCGGCCGGGGGGGGTGCCCCGCGGTGCGGG |
| | GGGGGCTGCGAGGGGAACAAAGGCTGCGTGCGGGGTGTGTGCGTGGGGGGGTGAG |
| | CAGGGGGTGTGGGCGCGTCGGTCGGGCTGCAACCCCCCCTGCACCCCCCTCCCCGA |
| | GTTGCTGAGCACGGCCCGGCTTCGGGTGCGGGGCTCCGTACGGGGCGTGGCGCGG |
| | GGCTCGCCGTGCCGGGGGGGGGGGGCGGCAGGTGGGGGTGCCGGGCGGGGCGGG |
| | GCCGCCTCGGGCCGGGGAGGGCTCGGGGGAGGGGCGCGGCGGCCCCCGGAGCGCC |
| | GGCGGCTGTCGAGGCGCGGCGAGCCGCAGCCATTGCCTTTTATGGTAATCGTGCGA |
| | GAGGGCGCAGGGACTTCCTTTGTCCCAAATCTGTGCGGAGCCGAAATCTGGGAGGC |
| | GCCGCCGCACCCCCTCTAGCGGGCGCGGGGCGAAGCGGTGCGGCGCCGGCAGGAA |
| | GGAAATGGGGGGGGAGGGCCTTCGTGCGTCGCCGCGCCGCCGTCCCCTTCTCCCTC |
| | TCCAGCCTCGGGGCTGTCCGCGGGGGGACGGCTGCCTTCGGGGGGGACGGGGCAG |
| | GGCGGGGTTCGGCTTCTGGCGTGTGACCGGCGGCTCTAGACAATTGTACTAACCTT |
| | CTTCTCTTTCCTCTCCTGACAGGGAGTTTAAACAGATAAGTTTGTACAAAAAAGAG |
| | AGGTGCCACCATGGGCTTTGTGCGACAGATTCAGCTGCTGCTGTGGAAGAACTGGA |
| | CCCTGCGGAAGCGGCAGAAAATCAGATTCGTGGTGGAACTCGTGTGGCCCCTGAG |
| | CCTGTTTCTGGTGCTGATCTGGCTGCGGAACGCCAATCCTCTGTACAGCCACCACG |
| | AGTGTCACTTCCCCAACAAGGCCATGCCTTCTGCCGGAATGCTGCCTTGGCTGCAG |
| | GGCATCTTCTGCAACGTGAACAACCCCTGCTTTCAGAGCCCCACACCTGGCGAAAG |
| | CCCTGGCATCGTGTCCAACTACAACAACAGCATCCTGGCCAGAGTGTACCGGGACT |
| | TCCAAGAGCTGCTGATGAACGCCCCTGAGTCTCAGCACCTGGGCAGAATCTGGACC |
| | GAGCTGCACATCCTGAGCCAGTTCATGGACACCCTGAGAACACACCCCGAGAGAA |
| | TCGCCGGCAGGGGCATCAGAATCCGGGACATCCTGAAGGACGAGGAAACCCTGAC |
| | ACTGTTCCTCATCAAGAACATCGGCCTGAGCGACAGCGTGGTGTACCTGCTGATCA |
| | ACAGCCAAGTGCGGCCCGAGCAGTTTGCTCATGGCGTGCCgGATCTCGCCCTGAAG |
| | GATATCGCCTGTTCTGAGGCCCTGCTGGAACGGTTCATCATCTTCAGCCAGCGGAG |
| | AGGCGCCAAGACCGTCAGATATGCCCTGTGCAGTCTGAGCCAGGGAACCCTGCAG |
| | TGGATCGAGGATACCCTGTACGCCAACGTGGACTTCTTCAAGCTGTTCCGGGTGCT |
| | GCCCACACTGCTGGATTCTAGATCCCAGGGCATCAACCTGAGAAGCTGGGGCGGC |
| | ATCCTGTCCGACATGAGCCCAAGAATCCAAGAGTTCATCCACCGGCCTAGCATGCA |
| | GGACCTGCTGTGGGTTACCAGACCTCTGATGCAGAACGGCGGACCCGAGACATTCA |
| | CCAAGCTGATGGGCATTCTGAGCGATCTGCTGTGCGGCTACCCTGAAGGCGGAGGA |
| | TCTAGAGTGCTGAGCTTCAATTGGTACGAGGACAACAACTACAAGGCCTTCCTGGG |
| | CATCGACTCCACCAGAAAGGACCCCATCTACAGCTACGACCGGCGGACAACCAGC |
| | TTCTGCAATGCCCTGATCCAGAGCCTGGAAAGCAACCCTCTGACCAAGATCGCTTG |
| | GAGGGCCGCCAAACCTCTGCTGATGGGAAAGATCCTGTACACCCCTGACAGCCCTG |
| | CCGCCAGAAGAATCCTGAAGAACGCCAACAGCACCTTCGAGGAACTGGAACACGT |
| | GCGCAAGCTGGTCAAGGCCTGGGAAGAAGTGGGACCTCAGATITGGTACTTCTTCG |
| | ACAATAGCACCCAGATGAACATGATCAGAGACACCCTGGGCAACCCTACCGTGAA |
| | GGACTTCCTGAACAGACAGCTGGGCGAAGAGGGCATTACCGCCGAGGCCATCCTG |
| | AACTTTCTGTACAAGGGCCCCAGAGAGTCCCAGGCCGACGACATGGCCAACTTCGA |
| | TTGGCGGGACATCTTCAACATCACCGACAGAACCCTGCGGCTGGTCAACCAGTACC |
| | TGGAATGCCTGGTGCTGGACAAGTTCGAGAGCTACAACGACGAGACACAGCTGAC |
| | CCAGAGAGCCCTGTCTCTGCTGGAAGAGAATATGTTCTGGGCTGGCGTGGTGTTCC |
| | CCGACATGTACCCTTGGACAAGCAGCCTGCCTCCTCACGTGAAGTACAAGATCCGG |
| | ATGGACATCGACGTGGTCGAAAAGACCAACAAGATCAAGGACCGGTACTGGGACA |
| | GCGGCCCTAGAGCTGATCCCGTGGAAGATTTTCGGTACATCTGGGGCGGATTCGCA |
| | TACCTGCAGGACATGGTGGAACAGGGAATCACACGGTCCCAGGTGCAGGCTGAAG |
| | CTCCTGTGGGAATCTACCTGCAGCAGATGCCTTATCCTTGCTTCGTGGACGACAGCT |
| | TCATGATCATCCTGAATCGGTGCTTCCCCATCTTCATGGTGCTGGCCTGGATCTACT |
| | CCGTGTCTATGACCGTGAAGTCCATCGTGCTGGAAAAAGAGCTGCGGCTGAAAGA |
| | GACACTGAAGAACCAGGGCGTGTCCAATGCCGTGATCTGGTGCACCTGGTTTCTGG |
| | ACAGCTTCTCCATTATGAGCATGAGCATCTTTCTGCTGACGATCTTCATCATGCACG |
| | GCCGaATCCTGCACTACAGCGACCCCTTTATCCTCTTCCTGTTCCTGCTGGCCTTCAG |
| | CACCGCTACAATCATGCTGTGTTTTCTGCTGTCCACCTTCTTCAGCAAGGCCTCTCT |
| | GGCCGCTGCTTGTAGCGGCGTGATCTACTTCACCCTGTACCTGCCTCACATCCTGTG |
| | CTTCGCATGGCAGGACAGAATGACCGCCGAGCTGAAGAAAGCTGTGTCCCTGCTG |
| | AGCCCTGTGGCCTTTGGCTTTGGCACCGAGTACCTCGTCAGATTTGAGGAACAAGG |
| | ACTGGGACTGCAGTGGTCCAACATCGGCAATAGCCCTACAGAGGGCGACGAGTTC |
| | AGCTTCCTGCTGTCTATGCAGATGATGCTGCTGGACGCCGCCGTGTATGGACTGCT |
| | GGCTTGGTATCTGGACCAGGTGTTCCCAGGCGATTACGGCACTCCTCTGCCTTGGT |
| | ATTTCCTGCTGCAAGAGAGCTACTGGCTCGGCGGCGAGGGATGTAGCACCAGAGA |
| | AGAAAGAGCCCTGGAAAAGACCGAGCCTCTGACCGAGGAAACAGAGGACCCTGA |
| | ACACCCAGAGGGCATCCACGATAGCTTTTTCGAGAGAGAACACCCCGGCTGGGTG |
| | CCAGGCGTGTGTGTGAAGAATCTGGTCAAGATITTCGAGCCCTGCGGCAGACCTGC |
| | CGTGGACAGACTGAACATCACCTTCTACGAGAACCAGATTACCGCCTTTCTGGGCC |
| | ACAACGGCGCTGGCAAGACAACCACAtTGAGCATCCTCACAGGCCTGCTGCCTCCA |
| | ACAAGCGGCACAGTTCTCGITGGCGGCAGAGACATCGAGACAAGCCTGGATGCCG |
| | TCAGACAGTCCCTGGGCATGTGCCCTCAGCACAACATCCTGTTTCACCACCTGACC |
| | GTGGCCGAGCACATGCTGTTTTATGCCCAGCTGAAGGGCAAGAGCCAAGAAGAGG |
| | CTCAGCTGGAAATGGAAGCCATGtTgGAGGACACCGGCCTGCACCACAAGAGAAAT |
| | GAGGAAGCCCAGGATCTGAGCGGCGGCATGCAGAGAAAACTGAGCGTGGCCATTG |
| | CCTTCGTGGGCGACGCCAAGGTTGTGATCCTGGATGAGCCTACAAGCGGCGTGGAC |
| | CCTTACAGCAGAAGATCCATCTGGGATCTGCTGCTGAAGTACAGAtcaGGCCGGACC |
| | ATCATCATGAGCACCCACCACATGGACGAGGCCGATCTGCTCGGAGACAGAATCG |
| | CCATCATTGCTCAGGGCAGACTGTACTGCAGCGGCACCCCACTGTTTCTGAAGAAC |
| | TGTTTCGGCACCGGACTGTATCTGACCCTCGTGCGGAAGATGAAGAACATCCAGTC |
| | TCAGCGGAAGGGCAGCGAGGGCACCTGTAGCTGTTCTAGCAAGGGCTTTAGCACC |
| | ACCTGTCCAGCTCACGTGGACGATCTGACCCCTGAACAGGTGCTGGATGGCGACGT |
| | GAACGAGCTGATGGACGTGGTGCTGCACCATGTGCCTGAGGCCAAGCTGGTGGAA |
| | TGCATCGGCCAAGAACTGATTTTTCTGCTCCCGAACAAGAACTTCAAGCACCGGGC |
| | CTACGCCAGCCTGTTCAGAGAGCTGGAAGAAACCCTGGCCGACCTGGGCCTGTCTA |
| | GCTTTGGCATCAGCGACACCCCTCTCGAAGAGATtTTCCTGAAAGTGACAGAGGAC |
| | AGCGATAGCGGCCCTCTGTTTGCTGGCGGAGCACAGCAAAAGCGCGAGAACGTGA |
| | ACCCTAGACACCCCTGTCTGGGCCCAAGAGAGAAAGCCGGACAGACCCCTCAGGA |
| | CAGCAATGTGTGCTCTCCTGGTGCTCCTGCCGCTCATCCTGAGGGACAACCTCCAC |
| | CTGAACCTGAGTGTCCTGGACCTCAGCTGAACACCGGAACACAGCTGGTTCTGCAG |
| | CACGTGCAGGCTCTGCTCGTGAAGAGATTCCAGCACACCATCAGAAGCCACAAGG |
| | ACTTTCTGGCCCAGATCGTGCTGCCCGCCACCTTTGTTTTTCTGGCTCTGATGCTGA |
| | GCATCGTGATCCCTCCATTCGGCGAGTACCCCGCTCTGACACTGCACCCTTGGATCT |
| | ACGGCCAGCAGTACACCTTTTTCTCCATGGACGAACCCGGCAGCGAGCAGTTCACA |
| | GTGCTGGCTGATGTCCTGCTGAACAAGCCCGGCTTCGGCAACCGGTGTCTGAAAGA |
| | AGGATGGCTGCCTGAGTACCCTTGCGGCAACAGCACACCTTGGAAAACCCCTAGCG |
| | TGTCCCCTAACATCACCCAGCTGTTCCAAAAGCAGAAATGGACCCAAGTGAACCCC |
| | TCTCCATCCTGCCGGTGCTCCACAAGGGAAAAGCTGACCATGCTGCCCGAGTGTCC |
| | AGAAGGCGCTGGCGGACTTCCTCCACCTCAGAGAACACAGAGATCCACCGAGATT |
| | CTCCAGGACCTGACCGACCGGAATATCAGCGACTTCCTGGTTAAGACATACCCCGC |
| | ACTGATCCGGTCCAGCCTGAAGTCCAAGTTCTGGGTCAACGAACAGAGATACGGC |
| | GGCATCAGCATCGGCGGAAAACTGCCTGTGGTGCCTATCACAGGCGAGGCCCTTGT |
| | GGGCTTTCTGTCCGATCTGGGGAGAATCATGAACGTGTCCGGCGGACCTATCACCA |
| | GGGAAGCCAGCAAAGAGATCCCCGATTTCCTGAAGCACCTGGAAACCGAGGACAA |
| | TATCAAAGTGTGGTTCAACAACAAAGGATGGCACGCCCTCGTGTCTTTTCTGAACG |
| | TGGCCCACAATGCCATCCTGCGGGCTAGCCTGCCTAAGGACAGAAGCCCTGAGGA |
| | ATACGGCATCACCGTGATCTCCCAGCCTCTGAATCTGACCAAAGAGCAGCTGAGCG |
| | AGATCACCGTGCTGACCACCTCTGTGGATGCTGTGGTGGCCATCTGCGTGATCTTC |
| | AGCATGAGCTTCGTGCCCGCCTCCTTCGTGCTGTACCTGATTCAAGAGAGAGTGAA |
| | CAAGAGCAAGCACCTCCAGTTCATCTCCGGGGTGTCCCCAACCACCTACTGGGTCA |
| | CCAATTTTCTGTGGGACATCATGAACTACAGCGTGTCAGCCGGCCTGGTCGTGGGC |
| | ATCTTTATCGGCTTTCAGAAGAAGGCCTACACGAGCCCCGAGAACCTGCCTGCTTT |
| | GGTTGCTCTGCTGCTCCTGTATGGCTGGGCCGTGATTCCCATGATGTACCCCGCCAG |
| | CTTTCTGTTTGACGTGCCCAGCACAGCCTACGTGGCCCTGTCTTGCGCCAATCTGTT |
| | CATCGGCATCAACAGCAGCGCCATCACATTCATCCTGGAACTGTTCGAGAACAACA |
| | GGACCCTGCTGCGGTTCAACGCCGTGCTGCGGAAACTGCTGATCGTGTTCCCTCAC |
| | TTCTGTCTCGGCAGAGGCCTGATCGACCTGGCTCTGTCTCAGGCCGTGACCGATGT |
| | GTACGCCAGATTTGGCGAGGAACACTCCGCCAATCCATTCCACTGGGACCTGATCG |
| | GCAAGAACCTGTTCGCCATGGTGGTGGAAGGCGTCGTGTACTTCCTGCTCACTCTG |
| | CTGGTGCAGAGACACTTTTTTCTGTOCCAATGGATCGCCGAGCCTACCAAAGAACC |
| | CATTGTGGACGAGGACGACGATGTGGCCGAGGAAAGACAGAGAATCATCACCGGC |
| | GGCAACAAGACCGATATCCTGAGACTGCACGAGCTGACAAAGATITACCCCGGCAC |
| | AAGCTCCCCAGCCGTGGATAGGCTTTGTGTGGGAGTTAGACCCGGCGAGTGCTTTG |
| | GCCTGCTGGGAGTTAATGGCGCCGGAAAGACCACCACCTTCAAGATGCTGACCGG |
| | CGACACCACAGTGACAAGCGGAGATGCTACAGTGGCCGGCAAGAGCATCCTGACC |
| | AACATCAGCGAAGTGCATCAGAACATGGGCTACTGCCCTCAGTTCGACGCCATCGA |
| | CGAACTGCTGACAGGCCGCGAACACCTGTATCTGTATGCCAGACTGAGAGGCGTGC |
| | CCGCTGAAGAGATCGAGAAGGTGGCCAACTGGTCCATCAAGTCTCTGGGCCTGAC |
| | AGTGTACGCCGACTGTCTGGCCGGAACATACAGCGGAGGAAACAAGCGGAAGCTG |
| | AGCACCGCCATTGCTCTGATCGGATGCCCACCTCTGGTCCTGCTGGATGAACCCAC |
| | CACCGGAATGGACCCCCAGGCTAGAAGAATGCTCTGGAACGTGATCGTGTCTATCA |
| | TCCGCGAGGGCAGAGCTGTGGTGCTGACCTCTCACAGCATGGAAGAGTGCGAGGC |
| | TCTGTGTACCCGGCTGGCCATTATGGTCAAGGGCGCCTTCAGATGCATGGGCACCA |
| | TTCAGCATCTGAAAAGCAAGTTCGGCGACGGCTACATCGTGACAATGAAGATCAA |
| | GAGCCCCAAGGACGACCTCCTGCCTGATCTGAACCCCGTGGAACAGTTTTTTCAGG |
| | GCAACTTCCCCGGCTCCGTGCAGCGGGAAAGACACTATAACATGCTGCAGTTTCAG |
| | GTGTCCTCCTCCAGCCTGGCTCGGATCTTTCAACTGCTGCTCTCTCACAAGGACAGC |
| | CTGCTGATTGAAGAGTACAGCGTGACACAGACCACACTCGACCAGGTTTTCGTGAA |
| | CTTCGCCAAGCAGCAGACCGAGAGCCACGACCTGCCTCTGCATCCTAGAGCCGCTG |
| | GTGCCTCTAGACAAGCTCAGGACTAAGCTTCCACTGGATTGTACAATTACATAAAA |
| | TAAAATATCTTTATTTTCATTACATCTGTGTGTTGGTTTTTTGTGTGCGCTACTtttttgag |
| | accggtttgtctggtcaaccaccgcggactccgtcgtcaggatcatagggcgctgttatctg |
| | ataaggcttatctggtctcatttt |
| SEQ ID NO: | ggtttgtctggtcaaccaccgcggactcagtggtgtacggtacaaacc |
| 9. attP-GA | |
| SEQ ID NO: | ggcttgtcgacgacggcggactccgtcgtcaggatcat |
| 10. attB-GA | |
| SEQ ID NO: | caattattgaaggcctccctaacggggggcctttttttgtttctggtcAcccgcttaacgat |
| 11. Bxb1 | cgttggctgaacaaacagacaatctggtctgtttgtattatggaaaatttttctgtataat |
| helper | agattcaacaaacagacaatctggtctgtttgtattatagctgtcaccggatgtgctttccggtctgat |
| plasmid | gagtccgtgaggacgaaacagcctctacaaataattttgtttaaTACTAGAGAAAGAGGAGAAATACTAGATG |
| | CGTGCACTGGTTGTTATTCGTCTGAGCCGTGTTACCGATGCAACCACCAGTCCGGA |
| | ACGTCAGCTGGAAAGCTGTCAGCAGCTGTGTGCACAGCGTGGTTGGGATGTTGTTG |
| | GTGTTGCCGAAGATCTGGATGTTAGCGGTGCAGTTGATCCGTTTGATCGTAAACGT |
| | CGTCCGAATCTGGCACGTTGGCTGGCATTTGAAGAACAGCCGTTTGATGTTATTGTT |
| | GCCTATCGTGTTGATCGTCTGACCCGTAGCATTCGTCATCTGCAGCAGCTGGTTCAT |
| | TGGGCAGAAGATCATAAAAAGCTGGTTGTGAGCGCAACCGAAGCACATTTTGATA |
| | CCACCACACCGTTTGCAGCAGTTGTTATTGCACTGATGGGCACCGTTGCACAGATG |
| | GAACTGGAAGCAATTAAAGAACGTAATCGTAGCGCAGCCCATTTTAACATTCGTGC |
| | AGGTAAATATCGTGGTAGCCTGCCTCCGTGGGGTTATCTGCCGACACGTGTTGATG |
| | GTGAATGGCGTCTGGTTCCGGATCCTGTTCAGCGTGAACGTATTCTGGAAGTTTATC |
| | ATCGTGTGGTGGATAATCATGAACCGCTGCATCTGGTTGCACATGATCTGAATCGT |
| | CGTGGTGTTCTGAGCCCGAAAGATTATTTTGCACAGCTGCAGGGTCGTGAACCGCA |
| | GGGTCGCGAATGGTCAGCAACCGCACTGAAACGTAGCATGATTAGCGAAGCAATG |
| | CTGGGTTATGCAACCCTGAATGGTAAAACCGTTCGTGATGATGATGGTGCACCGCT |
| | GGTTCGTGCAGAACCGATTCTGACACGTGAACAGCTGGAAGCACTGCGTGCCGAA |
| | CTGGTTAAAACCAGCCGTGCAAAACCGGCAGTTAGCACCCCGAGCCTGCTGCTGCG |
| | TGTTCTGTTTTGTGCAGTTTGTGGTGAACCGGCATACAAATTTGCCGGTGGTGGTCG |
| | TAAACATCCGCGTTATCGTTGTCGTAGCATGGGTTTTCCGAAACATTGTGGTAATG |
| | GTACAGTTGCAATGGCAGAATGGGATGCATTTTGCGAAGAACAGGTTCTGGATCTG |
| | CTGGGTGATGCCGAACGTCTGGAAAAAGTTTGGGTTGCAGGTAGCGATAGCGCAG |
| | TTGAACTGGCCGAAGTTAATGCAGAACTGGTTGATCTGACCAGCCTGATTGGTAGT |
| | CCGGCATATCGTGCCGGTAGTCCGCAGCGTGAAGCACTGGATGCACGTATTGCAGC |
| | ACTGGCAGCACGTCAAGAAGAATTAGAAGGTCTGGAAGCCCGTCCGAGCGGTTGG |
| | GAATGGCGTGAAACCGGTCAGCGTTTTGGTGATTGGTGGCGTGAGCAGGATACCGC |
| | AGCCAAAAATACCTGGCTGCGTAGTATGAATGTTCGCCTGACCTTTGATGTTCGCG |
| | GTGGTCTGACGCGTACCATTGATTTTGGCGATCTGCAAGAATATGAACAGCATCTG |
| | CGTCTGGGTAGCGTTGTTGAACGTCTGCATACCGGCATGAGCTAActcggtaccaaattccag |
| | aaaagaggcctcccgaaaggggggccttttttcgttttggtccaatggcggcgcgccatcgaatggtgcaaaa |
| | cctttcgcggtatggcatgatagcgcccggaagagagtcaattcagggtggtgaatatgagcccgaaacgtcg |
| | tacccaggcagaacgtgcaatggaaacccagggtaaactgattgcagcagcactgggtgttctgcgtgaaaaa |
| | ggttatgcaggttttcgtattgcagatgttccgggtgcagccggtgttagccgtggtgcacagagccatcatt |
| | ttccgaccaaactggaactgctgctggcaacctttgaatggctgtatgagcagattaccgaacgtagccgtgc |
| | acgtctggcaaaactgaaaccggaagatgatgttattcagcagatgctggatgatgcagcagaattttttctg |
| | gatgatgattttagcatcggcctggatctgattgttgcagcagatcgtgatccggcactgcgtgaaggtattc |
| | agcgtaccgttgaacgtaatcgttttgttgttgaagatatgtggctgggtgtgctggtgagccgtggtctgag |
| | ccgtgatgatgccgaagatattctgtggctgatttttaacagcgttcgtggtctggtagttcgtagcctgtgg |
| | cagaaagataaagaacgttttgaacgtgtgcgtaatagcaccctggaaattgcacgtgaacgttatgcaaaat |
| | tcaaacgttgataaggatcctaattggtaacgaatcagacaattgacggctcgagggagtagcatagggtttg |
| | cagaatccctgcttcgtccatttgacaggcacattatgcatcgatgataagctgtcaaacatgagcagatcct |
| | ctacgccggacgcatcgtggccggcatcaccggcgccacaggtgcggttgctggcgcctatatcgccgacatc |
| | accgatggggaagatcgggctcgccacttcgggctcatgagcaaatattttatctgaggtgcttcctcgctca |
| | ctgactcgctgcacgaggcagacctcaGGATCTccaggcatcaaataaaacgaaaggctcagtcgaaagac |
| | tgggcctttcgttttatctgttgtttgtcggtgaacgctctctactagagtcacactggctcaccttcggggg |
| | gcctttctgcgtttataGGATCTgcgaagttcctatactttctagagaataggaacttcgGATCTgtgcgcgg |
| | aacccctatttgtttatttttctaaatacattcaaatatgtatccgctcatgagacaataaccctgataaatg |
| | cttcaataatattgaaaaaggaagagtatgagtattcaacatttccgtgtcgcccttattcccttttttgcgg |
| | cattttgccttcctgtttttgctcacccagaaacgctggtgaaagtaaaagatgctgaagatcagttgggtgc |
| | acgagtgggttacatcgaactggatctcaacagcggtaagatccttgagagttttcgccccgaagaacgtttt |
| | ccaatgatgagcacttttaaagttctgctatgtggcgcggtattatcccgtattgacgccgggcaagagcaac |
| | tcggtcgccgcatacactattctcagaatgacttggttgagtactcaccagtcacagaaaagcatcttacgga |
| | tggcatgacagtaagagaattatgcagtgctgccataaccatgagtgataacactgcggccaacttacttctg |
| | acaacgatcggaggaccgaaggagctaaccgcttttttgcacaacatgggggatcatgtaactcgccttgatc |
| | gttgggaaccggagctgaatgaagccataccaaacgacgagcgtgacaccacgatgcctgtagcaatggcaac |
| | aacgttgcgcaaactattaactggcgaactacttactctagcttcccggcaacaattaatagactggatggag |
| | gcggataaagttgcaggaccacttctgcgctcggcccttccggctggctggtttattgctgataaatctggag |
| | ccggtgagcgtggttctcgcggtatcattgcagcactggggccagatggtaagccctcccgtatcgtagttat |
| | ctacacgacggggagtcaggcaactatggatgaacgaaatagacagatcgctgagataggtgcctcactgatt |
| | aagcattggtaactgtcagaccaagtttactcatatatactttagattgatttaaaacttcatttttaattta |
| | aaaggatctaggtgaagatcctttttgataatcTA |
| | TGGACAGTTTTCCCTTTGATATCTAACGGTGAACAGTTGTTCTACTTTTGTTTGTTAG |
| | TCTTGATGCTTCACTGATAGATACAAGAGCCATAAGAACCTCAGATCCTTCCGTAT |
| | TTAGCCAGTATGTTCTCTAGTGTGGTTCGTTGTTTTTGCGTGAGCCATGAGAACGAA |
| | CCATTGAGATCATGCTTACTTTGCATGTCACTCAAAAATTTTGCCTCAAAACTGGTG |
| | AGCTGAATTTTTGCAGTTAAAGCATCGTGTAGTGTTTTTCTTAGTCCGTTACGTAGG |
| | TAGGAATCTGATGTAATGGTTGTTGGTATTTTGTCACCATTCATTTTTATCTGGTTGT |
| | TCTCAAGTTCGGTTACGAGATCCATTTGTCTATCTAGTTCAACTTGGAAAATCAACG |
| | TATCAGTCGGGCGGCCTCGCTTATCAACCACCAATTTCATATTGCTGTAAGTGTTTA |
| | AATCTTTACTTATTGGTTTCAAAACCCATTGGTTAAGCCTTTTAAACTCATGGTAGT |
| | TATTTTCAAGCATTAACATGAACTTAAATTCATCAAGGCTAATCTCTATATTTGCCT |
| | TGTGAGTTTTCTTTTGTGTTAGTTCTTTTAATAACCACTCATAAATCCTCATAGAGTA |
| | TTTGTTTTCAAAAGACTTAACATGITCCAGATTATATTTTATGAATTTTTTTAACTGG |
| | AAAAGATAAGGCAATATCTCTTCACTAAAAACTAATTCTAATTTTTCGCTTGAGAA |
| | CTTGGCATAGTTTGTCCACTGGAAAATCTCAAAGCCTTTAACCAAAGGATTCCTGA |
| | TTTCCACAGTTCTCGTCATCAGCTCTCTGGTTGCTTTAGCTAATACACCATAAGCAT |
| | TTTCCCTACTGATGTTCATCATCTGAACGTATTGGTTATAAGTGAACGATACCGTCC |
| | GTTCTTTCCTTGTAGGGTTTTCAATCGTGGGGTTGAGTAGTGCCACACAGCATAAA |
| | ATTAGCTTGGTTTCATGCTCCGTTAAGTCATAGCGACTAATCGCTAGTTCATTTGCT |
| | TTGAAAACAACTAATTCAGACATACATCTCAATTGGTCTAGGTGATTTTAATCACT |
| | ATACCAATTGAGATGGGCTAGTCAATGATAATTACTAGTCCTTTTCCTTTGAGTTGT |
| | GGGTATCTGTAAATTCTGCTAGACCTTTGCTGGAAAACTTGTAAATTCTGCTAGACC |
| | CTCTGTAAATTCCGCTAGACCTTTGTGTGTTTTTTTTGTTTATATTCAAGTGGTTATA |
| | ATTTATAGAATAAAGAAAGAATAAAAAAAGATAAAAAGAATAGATCCCAGCCCTG |
| | TGTATAACTCACTACTTTAGTCAGTTCCGCAGTATTACAAAAGGATGTCGCAAACG |
| | CTGTTTGCTCCTCTACAAAACAGACCTTAAAACCCTAAAGGCTTAAGTAGCACCCT |
| | CGCAAGCTCGGGCAAATCGCTGAATATTCCTTTTGTCTCCGACCATCAGGCACCTG |
| | AGTCGCTGTCTTTTTCGTGACATTCAGTTCGCTGCGCTCACGGCTCTGGCAGTGAAT |
| | GGGGGTAAATGGCACTACAGGCGCCTTTTATGGATTCATGCAAGGAAACTACCCAT |
| | AATACAAGAAAAGCCCGTCACGGGCTTCTCAGGGCGTTTTATGGCGGGTCTGCTAT |
| | GTGGTGCTATCTGACTTTTTGCTGTTCAGCAGTTCCTGCCCTCTGATTTTCCAGTCTG |
| | ACCACTTCGGATTATCCCGTGACAGGTCATTCAGACTGGCTAATGCACCCAGTAAG |
| | GCAGCGGTATCA |

What is claimed is:

1. An engineered bacterial cell comprising:
(a) a Rep gene encoding a bacterial replication protein integrated into the bacterial genome;
(b) a circular DNA vector comprising:
(i) a coding sequence; and
(ii) a replication origin that is dependent on the replication protein.

2. The engineered bacterial cell of claim 1, wherein the replication origin is less than 50 base pairs in length.

3. The engineered bacterial cell of claim 1 or 2, wherein the replication origin and replication protein are from a ColE2-related plasmid.

4. The engineered bacterial cell of claim 3, wherein the ColE2-related plasmid is ColE2-P9.

5. The engineered bacterial cell of any one of claims 1 to 4, wherein the Rep gene is operatively coupled to a first inducible promoter.

6. The engineered bacterial cell of claim 5, wherein the first inducible promoter is a T7 RNA polymerase-dependent promoter.

7. The engineered bacterial cell of any one of claims 1 to 6, further comprising a gene encoding T7 RNA polymerase (T7RNAP) integrated into the bacterial genome.

8. The engineered bacterial cell of claim 7, wherein the T7RNAP gene is operatively coupled to a second inducible promoter.

9. The engineered bacterial cell of claim 8, wherein the second inducible promoter is Ptac.

10. The engineered bacterial cell of any one of claims 1 to 9, further comprising a gene encoding an exogenous restriction enzyme integrated into the bacterial genome.

11. The engineered bacterial cell of claim 10, wherein the gene encoding the exogenous restriction enzyme is operatively coupled to a third inducible promoter.

12. The engineered bacterial cell of claim 11, wherein the third inducible promoter is Pbad.

13. The engineered bacterial cell of claim 11 or 12, wherein the bacterial genome does not comprise a recognition sequence for the exogenous restriction enzyme.

14. The engineered bacterial cell of any one of claims 1 to 13, wherein the coding sequence encodes a therapeutic gene or nucleic acid.

15. The engineered bacterial cell of any one of claims 1 to 14, wherein the coding sequence is a eukaryotic sequence.

16. The engineered bacterial cell of any one of claims 1 to 15, wherein the replication origin is the only bacterial sequence in the circular DNA vector.

17. The engineered bacterial cell of any one of claims 1 to 16, wherein the engineered bacterial cell comprises at least 20 copies of the circular DNA vector.

18. The engineered bacterial cell of any one of claims 1 to 17, wherein the engineered bacterial cell is capable of maintaining the circular DNA vector through at least 20 rounds of cell division.

19. The engineered bacterial cell of any one of claims 1 to 18, wherein the engineered bacterial cell does not comprise any extragenomic circular DNA molecules other than one or more copies of the circular DNA vector.

20. A culture comprising a plurality of engineered bacterial cells of any one of claims 1-19, wherein the mean numbers of copies of the circular DNA vector per engineered bacterial cell is at least 10.

21. The culture of claim 20, comprising at least 10⁷ engineered bacterial cells.

22. An engineered bacterial cell comprising:
(a) a Rep gene encoding a bacterial replication protein integrated into the bacterial genome;
(b) a plasmid comprising:
(i) a first segment comprising a coding sequence and a replication origin that is dependent on the bacterial replication protein, wherein the first segment does not comprise a selectable marker; and
(ii) a second segment comprising a selectable marker;
wherein the first segment is flanked by recognition sequences for at least one exogenous restriction enzyme or exogenous recombinase.

23. The engineered bacterial cell of claim 22, wherein the recognition sequences flanking the first segment are the same.

24. The engineered bacterial cell of claim 22, wherein the recognition sequences flanking the first segment are different.

25. The engineered bacterial cell of claim 22, wherein the second segment further comprises a replication origin, wherein the replication origin in the second segment is orthologous to the replication origin in the first segment.

26. The engineered bacterial cell of claim 22, wherein the replication origin is less than 50 base pairs in length.

27. The engineered bacterial cell of claim 22, wherein the replication origin and replication protein are from a ColE2-related plasmid.

28. The engineered bacterial cell of claim 22, wherein the ColE2-related plasmid is ColE2-P9.

29. The engineered bacterial cell of any one of claims 22-27, wherein the Rep gene is operatively coupled to a first inducible promoter.

30. The engineered bacterial cell of claim 29, wherein the first inducible promoter is a T7 RNA polymerase-dependent promoter.

31. The engineered bacterial cell of any one of claims 22-30, further comprising a gene encoding T7 RNA polymerase (T7RNAP) integrated into the bacterial genome.

32. The engineered bacterial cell of claim 31, wherein the T7RNAP gene is operatively coupled to a second inducible promoter.

33. The engineered bacterial cell of claim 32, wherein the second inducible promoter is Ptac.

34. The engineered bacterial cell of any one of claims 22 to 33, further comprising a gene encoding the exogenous restriction enzyme or exogenous recombinase integrated into the bacterial genome.

35. The engineered bacterial cell of claim 34, wherein the gene encoding the exogenous restriction enzyme or exogenous recombinase is operatively coupled to a third inducible promoter.

36. The engineered bacterial cell of claim 35, wherein the third inducible promoter is Pbad.

37. The engineered bacterial cell of any one of claims 22 to 36, wherein the bacterial genome does not comprise a recognition sequence for the exogenous restriction enzyme or the exogenous recombinase.

38. The engineered bacterial cell of any one of claims 22 to 37, wherein the coding sequence encodes a therapeutic gene or nucleic acid.

39. The engineered bacterial cell of any one of claims 1 to 19, wherein the coding sequence is a eukaryotic sequence.

40. The engineered bacterial cell of any one of claims 22 to 39, further comprising the exogenous restriction enzyme or exogenous recombinase.

41. A method of making a circular DNA vector, the method comprising:
(a) contacting a plasmid within a bacterial cell with an exogenous restriction enzyme to excise a first segment from the plasmid, wherein the first segment is flanked by recognition sequences for the exogenous restriction enzyme, and wherein the first segment comprises a coding sequence and a replication origin dependent on a bacterial replication protein, thereby generating a linear DNA fragment comprising a 5′ end and a 3′ end with complimentary overhangs; and
(b) ligating the 5′ and the 3′ end of the linear DNA fragment together to generate the circular DNA vector.

42. The method of claim 41, wherein before step (a), the plasmid comprises a second segment comprising a selectable marker.

43. The method of claim 42, wherein the second segment further comprises a replication origin, wherein the replication origin in the second segment is orthologous to the replication origin in the first segment.

44. The method of any one of claims 41 to 43, wherein the first segment does not comprise a selectable marker.

45. The method of any one of claims 41 to 44, wherein contacting the plasmid within the cell with the exogenous restriction enzyme comprises inducing expression of the exogenous restriction enzyme within the cell.

46. The method of claim 45, wherein a gene encoding the exogenous restriction enzyme is integrated into the bacterial genome operatively coupled to an inducible promoter.

47. The method of claim 46, wherein the inducible promoter is Pbad, and wherein inducing expression of the exogenous restriction enzyme within the cell comprises providing arabinose to the cell.

48. The method of any one of claims 41 to 44, wherein contacting the plasmid within the cell with the exogenous restriction enzyme comprises introducing the exogenous restriction enzyme into the cell from outside of the bacterial cell.

49. The method of any one of claims 41 to 48, wherein the ligating is performed by an exogenous ligase.

50. The method of claim 49, wherein the exogenous ligase is expressed from a gene integrated into the bacterial genome.

51. The method of claim 50, wherein the exogenous ligase is introduced into the bacterial cell from outside the bacterial cell.

52. The method of any one of claims 41 to 51, wherein the bacterial cell comprises a Rep gene encoding the bacterial replication protein integrated into the genome.

53. The method of claim 52, wherein the Rep gene is operatively coupled to an inducible promoter capable of expressing the bacterial replication protein at a first expression level and a second expression level, wherein the first expression level is lower than the second expression level.

54. The method of claim 53, wherein the first expression level of the bacterial replication protein causes the replication origin to be maintained at a first copy number, and wherein the second expression level of the bacterial replication protein causes the replication origin to be maintained at a second copy number, wherein the first copy number is below 5, 10, 15, 20, or 50 copies per cell and the second copy number is at least 20, 50, 100, or 200 copies per cell.

55. The method of claim 53, wherein the bacterial replication gene is expressed at the first expression level before step (b) and is not expressed at the second expression level before step (b).

56. The method of claim 54 or 55, wherein the bacterial replication gene is expressed at the second expression level after step (b).

57. The method of any one of claims 53 to 56, wherein the inducible promoter is a PT7 dependent on T7 RNA polymerase.

58. The method of any one of claims 41 to 57, wherein the bacterial cell comprises a gene encoding T7 RNA polymerase integrated into the genome (T7R NAP).

59. The method of claim 58, wherein the T7RNAP gene is operatively coupled to an inducible promoter.

60. The method of claim 59, wherein the inducible promoter is P_{tac}.

61. The method of claim 60, wherein the second segment of the plasmid further comprises a LacI gene encoding a lactose inhibitor protein capable of suppressing expression from the P_{tac} promoter, and wherein expression of the bacterial replication gene is maintained at or below the first expression level by expression of the lactose inhibitor protein.

62. The method of claim 61, wherein after step (b) expression of the lactose inhibitor protein is reduced, thereby inducing the bacterial replication gene to be expressed at the second expression level and causing the circular DNA vector to be maintained at the second copy number.

63. The method of any one of claims 41 to 62, further comprising culturing the cell under conditions in which the selectable marker on the plasmid is not needed for continued growth, thereby generating a population of progeny of the bacterial cell that lack the selectable marker.

64. The method of claim 63, wherein the population maintains the circular DNA vector at an average copy number of at least 20 copies per cell after at least 50 doublings.

65. The method of claim 63 or 64, further comprising purifying the circular DNA vector.

66. The method of any one of claims 41 to 65, wherein the replication origin is less than 50 base pairs in length.

67. The method of any one of claims 41 to 66, wherein the replication origin and replication protein are from a ColE2-related plasmid.

68. The method of claim 67, wherein the ColE2-related plasmid is ColE2-P9.

69. A method of making a circular DNA vector, the method comprising:
(a) obtaining the engineered bacterial cell of any one of claims 22 to 40;
(b) contacting the plasmid with the exogenous restriction enzyme to excise the first segment of the plasmid, thereby generating a linear DNA fragment flanked by complementary overhangs; and
(c) self-ligating the linear DNA fragment to generate the circular DNA vector.

70. A method of making a circular DNA vector, the method comprising:
(a) obtaining the engineered bacterial cell of any one of claims 22 to 40;
(b) contacting the plasmid with the exogenous recombinase that recognizes the recognition sequences flanking the first segment.

71. A pharmaceutical composition comprising:
(a) a circular DNA vector produced by any of the methods in claims 41-70; and
(b) a suitable carrier for use in delivering the pharmaceutical composition to a subject.

72. An engineered bacterial cell comprising a circular DNA vector comprising a coding sequence and a replication origin that is less than 50 base pairs in length, wherein the circular DNA vector lacks a selectable marker.

73. The engineered bacterial cell of claim 72, wherein the engineered bacterial cell does not comprise any extragenomic DNA molecules other than one or more copies of the circular DNA vector.

74. The engineered bacterial cell of claim 72 or 73, wherein the engineered bacterial cell does not comprise a gene encoding a selectable marker.

75. The engineered bacterial cell of any one of claims 72 to 74, wherein the engineered bacterial cell does not comprise a selectable marker on an extragenomic DNA molecule.

76. The engineered bacterial cell of any one of claims 72 to 75, wherein the replication origin is from a ColE2-P9 plasmid.

77. The engineered bacterial cell of any one of claims 72 to 76, further comprising a Rep gene encoding a bacterial replication protein that recognizes the origin of replication.

78. The engineered bacterial cell of claim 77, wherein the Rep gene is from a ColE2-P9 plasmid.

79. The engineered bacterial cell of claim 77 or 78, wherein the Rep gene is integrated into the bacterial genomic.

80. The engineered bacterial cell of any one of claims 77 to 79, wherein the Rep gene is operatively coupled to an inducible promoter.

81. The engineered bacterial cell of any one of claims 72 to 80, wherein the circular DNA vector further comprises a recombination site.

82. The engineered bacterial cell of any one of claims 72 to 81, wherein the circular DNA vector does not comprise any bacterial [or other prokaryotic or phage] sequence other than the origin of replication and, when present, the recombination site.

83. The engineered bacterial cell of claim 81, wherein the origin of replication and recombination site together are no more than 90 base pairs in length.

84. The engineered bacterial cell of any one of claims 72 to 83, further comprising a gene encoding a recombinase.

85. The engineered bacterial cell of any one of claims 72 to 83, wherein the bacterial cell comprises at least 10 copies of the circular DNA vector.

86. An engineered bacterial cell comprising a plasmid that comprises:
(a) a first segment comprising a coding sequence and a replication origin that is less than 50 base pairs in length, wherein the first segment does not comprise a selectable marker; and
(b) a second segment comprising a selectable marker;
wherein the first segment is flanked by recognition sequences for an exogenous recombinase.

87. The engineered bacterial cell of claim 86, further comprising a gene encoding a Rep gene encoding a bacterial replication protein that recognizes the replication origin.

88. The engineered bacterial cell of claim 87, wherein the Rep gene is integrated into the bacterial genome.

89. The engineered bacterial cell of claim 87 or 88, wherein the Rep gene is operatively coupled to a first inducible promoter.

90. The engineered bacterial cell of any one of claims 87 to 89, wherein the replication origin and replication protein are from a ColE2-related plasmid.

91. The engineered bacterial cell of any one of claims 87 to 90, wherein the ColE2-related plasmid is ColE2-P9.

92. The engineered bacterial cell of any one of claims 86 to 91, further comprising a gene encoding the exogenous recombinase.

93. The engineered bacterial cell of claim 92, wherein the gene encoding the exogenous recombinase is integrated into the bacterial genomic.

94. The engineered bacterial cell of claim 92, wherein the gene encoding the exogenous recombinase is on a plasmid or bacterial artificial chromosome.

95. The engineered bacterial cell of any one of claims 92 to 94, wherein the gene encoding the exogenous recombinase is operatively coupled to a second inducible promoter.

96. The engineered bacterial cell of claim 95, wherein the second inducible promoter is a cuminic acid-inducible promoter.

97. The engineered bacterial cell of any one of claims 92 to 96, wherein the recombinase is Bxb1.

98. The engineered bacterial cell of claim 97, wherein the recognition sequences comprise attP-GA and attB-GA.

99. A method of making a circular DNA vector comprising inducing recombination of the plasmid in the engineered bacterial cell of any one of claims 86 to 98.

100. The method of claim 99, wherein inducing recombination of the plasmid comprises inducing expression of the exogenous recombinase in the engineered bacterial cell.

101. A method of producing a circular DNA vector, the method comprising inducing recombination of a plasmid in an engineered bacterial cell, wherein:
(a) the plasmid comprises:
(ii) a first segment comprising a coding sequence and a replication origin that is less than 50 base pairs in length, wherein the first segment does not comprise a selectable marker, wherein the first segment is flanked by recognition sequences for an exogenous recombinase; and
(ii) a second segment comprising a selectable marker; and
(b) the engineered bacterial cell comprises a gene encoding the exogenous recombinase;
wherein the inducing causes recombination of the plasmid, thereby producing the circular DNA vector comprising the first segment.

102. The method of claim 101, wherein the engineered bacterial cell further comprises a Rep gene encoding a bacterial replication protein that recognizes the origin of replication.

103. The method of claim 101 or 102, wherein the Rep gene is integrated into the bacterial genome.

104. The method of claim 102 or 103, wherein the Rep gene is operatively coupled to a first inducible promoter.

105. The method of any one of claims 101 to 104, wherein the replication origin is a ColE2-P9 replication origin.

106. The method of any one of claims 102 to 105, wherein the Rep gene is a ColE2-P9 Rep gene.

107. The method of any one of claims 101 to 106, wherein the exogenous recombinase is on a plasmid or bacterial artificial chromosome.

108. The method of any one of claims 101 to 107, wherein the gene encoding the exogenous recombinase is operatively coupled to a second inducible promoter.

109. The method of claim 108, wherein the inducing recombination of the plasmid comprises inducing expression of the gene encoding the exogenous recombinase.

110. The method of any one of claims 101 to 108, wherein the inducing recombination of the plasmid comprises introducing the plasmid into the engineered bacterial cell, wherein the exogenous recombinase is expressed in the engineered bacterial cell at the time of the introducing.

111. The method of claim 110, wherein the exogenous recombinase is expressed at a non-induced level at the time of the introducing.

112. The method of any one of claims 101 to 111, wherein the exogenous recombinase is Bxb1 and the recognition sequences comprise attP-GA and attB-GA.

113. The method of claim 112, wherein the gene encoding Bxb1 is operatively coupled to a cuminic acid-inducible promoter, the engineered bacterial cell is maintained in the absence of cuminic acid at the time of the introducing, and the Bxb1 is expressed at a non-induced level at the time of the introducing.

114. A pharmaceutical composition comprising:
(a) a circular DNA vector produced by any of the methods in claims 99-113; and
(b) a suitable carrier for use in delivering the pharmaceutical composition to a subject.

115. A circular DNA vector comprising:
(a) a eukaryotic promoter;
(b) a eukaryotic coding sequence; and
(c) a bacterial replication origin that is less than 50 bp in length,
wherein the circular DNA vector lacks a selectable marker.

116. The circular DNA vector of claim 115, wherein the 3′ end of the eukaryotic coding sequence is linked to the 5′ end of the promoter by a sequence comprising the bacterial origin of replication, wherein the sequence comprising the bacterial origin of replication is less than 100 bp in length.1

117. A pharmaceutical composition comprising:
(d) the circular DNA vector of any one of claim 115 or 116; and
(e) a suitable carrier for use in delivering the pharmaceutical composition to a subject.

118. A host cell comprising the circular DNA vector of any one of claim 115 or 116.

119. The host cell of claim 118, wherein the host cell is a mammalian cell.

120. The host cell of claim 118 or 119, wherein the host cell is a human cell.

121. The host cell of any one of claims 117-120, wherein the host cell is isolated in vitro.
